(19) United States
(12) Patent Application Publication
(10) Pub. No.: US 2009/0233993 A1
(43) Pub. Date: Sep. 17, 2009

(54) COMPOSITIONS AND METHODS FOR INHIBITING GSK3 ACTIVITY AND USES THEREOF

(75) Inventors: Huaxi Xu, La Jolla, CA (US); Limin Li, Potomac, MD (US); Francesca-Fang Liao, La Jolla, CA (US); Yun-wu Zhang, La Jolla, CA (US); Wu-bo Li, Gaithersburg, MD (US); Paul Greengard, New York, NY (US); Stanley N. Cohen, Stanford, CA (US)

Correspondence Address: PATENT CORRESPONDENCE;ARNALL GOLDEN GREGORY LLP, 171 17TH STREET NW, SUITE 2100, ATLANTA, GA, 30363, US

(73) Assignee: Burnham Institute for Medical Research, La Jolla, CA (US)

(21) Appl. No.: 12/399,850

(22) Filed: Mar. 6, 2009

Related U.S. Application Data
(60) Provisional application No. 61/034,302, filed on Mar. 6, 2008.

(51) Int. Cl. A61K 31/7088; C07H 21/00; C12N 15/74; C12P 21/02; C07K 2/00; C12N 5/06; A61P 25/28; G01N 33/567

(52) U.S. Cl. 514/44 R; 536/23.1; 435/320.1; 435/325; 435/69.1; 530/350; 435/375; 435/7.21

(57) ABSTRACT

Disclosed herein are compositions and methods relating to peptides. The peptides can inhibit amyloid beta (Aβ) generation and reduce GSK-3 activities. Further provided are compositions and methods for treating or preventing, for example, Alzheimer's disease, cancer, and diabetes.

### CROSS-REFERENCE TO RELATED APPLICATIONS

This application claims benefit of U.S. Provisional Application No. 61/034,302, filed Mar. 6, 2008. Application No. 61/034,302, filed Mar. 6, 2008, is hereby incorporated herein by reference in its entirety.

### STATEMENT REGARDING FEDERALLY SPONSORED RESEARCH

This invention was made with government support under Grants R01 AG021173, R01 NS046673, R01 AG030197, R01 NS054880, and P01 AG009464 from National Institutes of Health; and AOA 90AZ2791 from US Department of Health and Human Services. The government has certain rights in the invention.

### BACKGROUND

Alzheimer's disease (AD) is characterized by extracellular neuritic plaques, intracellular neurofibrillary tangles (NFTs), synaptic dysfunctions and neural degeneration in vulnerable brain regions including frontal tempo cortex and hippocampus that are essential for cognition, learning and memory (Tanzi, R. E., et al. 2005). Neuritic plaques are composed of aggregates of heterogeneous β-amyloid (Aβ) peptides which are produced through sequential proteolysis of the β-amyloid precursor protein (APP) (Greenfield, J. P., et al. 2000). APP belongs to type I transmembrane glycoprotein. Although the physiological functions of APP have not been determined, the intracellular domain of APP (AICD) has been implicated in regulation of gene expression (Cao, X., et al. 2001; Gao, Y., et al. 2001; Baek, S. H., et al. 2002). The route of APP to Aβ has been well defined. APP can be cleaved by three types of proteases, which are designated α-, β-, and γ-secretases (De Strooper, B. 2003). Cleavage by β- and γ-secretase creates the NH₃- and COOH-terminal ends of the Aβ peptide, respectively, and releases Aβ, whereas α-secretase cleaves APP within the Aβ sequence and precludes Aβ formation to adopt a non-amyloidogenic fate to release soluble APPα (sAPPα) fragment (FIG. 1). The neuroprotective effects of sAPPα have been proposed and supported by a number of in vitro studies (Mattson, M. P., et al. 1993). The other hallmark of AD is the NFTs, whose major components are hyperphosphorylated microtubule associated protein tau (Lee, V. M., et al. 2001). Multiple protein kinases, including but not limited to GSK3, CDK5, Akt and PKA, can phosphorylate tau. While NFTs are also found in other neurodegenerative disorders, such as Pick's disease (PiD) and corticobasal degeneration (CBD), amyloid plaques are specific to AD and several lines of evidence have implicated a central role for Aβ in AD pathogenesis using cellular and animal models.

Genetic and biochemical studies on familial forms of AD (FAD) have shown that a variety of pathogenic mutations in the APP gene and in genes encoding presenilins 1 and 2 (PS1 and PS2) increase the production of the highly amyloidogenic Aβ42 which is also known to be elevated in sporadic (non-familial) forms of AD, suggesting that accumulation of Aβ is a common mechanism underlying all forms of AD. In addition, transgenic mice with increased Aβ levels show some neurodegenerative changes and cognitive defects similar to AD. Furthermore, intracellular Aβ immunoreactivity and appearance of amyloid plaques precede formation of NFTs in the triple transgenic AD mouse model harboring PS1_{M146V}, APP_{Swe}, and tau_{P301L} transgenes (Oddo, S., et al. 2003); and the clearance of intracellular and extracellular Aβ reverses tau aggregates in the mice (Oddo, S., et al. 2004). These data suggest a causal role of Aβ in pathogenesis and progression of AD, and indicate that reduction of Aβ, by inhibiting β- and γ-secretase, would be an ideal strategy for the AD therapy.

Thus far, a single cDNA encoding an aspartyl protease has been unequivocally confirmed as the β-secretase, or BACE1 (Vassar, R., et al. 1999; Hussain, I., et al. 1999; Yan, R., et al. 1999; Sinha, S., et al. 1999; Lin, X., et al. 2000), which contains a characteristic single type I transmembrane domain near its C terminus.

γ-secretase is a high-molecular-mass, membrane-bound protein complex, composed of at least four molecules, presenilin (PS1 and PS2) (Sherrington, R., et al. 1995; Rogaev, E. I., et al. 1995), nicastrin (Yu, G., et al. 2000), Pen-2 (Francis, R., et al. 2002) and Aph-1 (Francis, R., et al. 2002; Goutte, C., et al. 2002). The γ-secretase activity is abolished by deficiency in PS1 or any one of the components. Reconstitution of these four components in yeast and Spodoptera frugiperda (sf9) cells can achieve modest cleavage activity (Edbauer, D., et al. 2003; Kimberly, W. T., et al. 2003; Takasugi, N., et al. 2003). However, it is plausible that there are other interacting partners of the four molecules. The recent finding of TMP21 as yet another presenilin complex component (Chen, F., et al. 2006) underlines the existence of other γ-secretase components or regulators to modulate γ-secretase in terms of substrate specificity and relative activities at different cleavage sites to generate Aβ40 or Aβ42. γ-secretase cleavage occurs within the transmembrane domain of APP and many other membrane proteins such as APP homologues APLP1 and APLP2, the transmembrane Notch receptors, and cadherin (Naruse, S., et al. 1998; De Strooper, B., et al. 1998; Struhl, G., et al. 2000). In addition to a role in γ-secretase processing of membrane proteins, PS1 and PS2 are thought to mediate a number of cellular functions, some of which are affected by FAD-linked mutations in PS. These include a role for PS in calcium homeostasis, cell adhesion, apoptosis, and synaptic plasticity (Sisodia, S. S., et al. 1999). In addition, mounting evidence suggests a role of PS1 in trafficking of APP and other membrane proteins. It has been found that PS1 is required for maturation and cell surface transport of nicastrin (Leem, J. Y., et al. 2002; Yang, D. S., et al. 2002). In addition, the secretion rate of APP in neurons derived from PS1^{-/-} embryos have been reported to be altered (Naruse, S., et al. 1998); and the accumulation of APP and Notch on the surface of cells expressing dominant negative mutants of PS1 has been found increased (Capell, A., et al. 2000; Kim, S. H., et al. 2001). Furthermore, it is possible to inhibit Notch1 processing without any loss of γ-secretase cleavage of APP, and vice versa, by the use of selective inhibitors or the expression of PS variants (Capell, A., et al. 2000; Kulic, L., et al. 2000; Petit, A., et al. 2001).

The fact that APP metabolism/Aβ generation is highly regulated via various signal transduction pathways, e.g., various protein kinases and phosphatases (Buxbaum, J. D., et al. 1994; Mills, J., et al. 1999) and insulin (Gasparini, L., et al. 2001), strongly indicates the involvement of multiple regulatory factors/proteins. The effects of these compounds on APP metabolism may also be mediated by mechanisms not involving direct regulation of enzymatic activity. For example, compounds that stimulate APP transport leading to an increased egress of APP from cellular compartments where β- and/or γ-secretase are highly active should reduce Aβ formation (Gasparini, L., et al. 2001; Xu, H., et al. 1995; Xu, H., et al. 1996; Greenfield, J. P., et al. 2002). In addition, alternations in other cellular processes, such as cholesterol levels and calcium homeostasis have been reported to affect APP metabolism (Buxbaum, J. D., et al. 1994; Puglielli, L., et al. 2001; Chen, M., et al. 1999; Golde, T. E., et al. 2001). Identification of genes encoding those proteins is expected to be instrumental in developing anti-amyloid therapies in addition to interventions aimed directly at inhibiting the secretase catalytic cores.

GSK3 is a serine/threonine kinase that was originally identified as a key enzyme functioning in the metabolism of glycogen (Embi, N., et al. 1980) but is now known to play roles in a variety of cellular functions including cell adhesion, cell-division, transcription (Frame, S., et al. 2001), and tau phosphorylation (Anderton, B. H., et al. 2001). Many tau serine and threonine sites that are found phosphorylated in NFTs can be phosphorylated by GSK3 (Sperber, B. R., et al. 1995; Hanger, D. P., et al. 1992; Lovestone, S., et al. 1994). Recent studies have shown that GSK-3β interacts with and binds presenilin in normal human brains and cells (Takashima, A., et al. 1998; Tesco, G., et al. 2000). CDK5 has been reported as a crucial regulator of neuronal migration in the developing central nervous system (Paglini, G., et al. 2001) and cytoskeletal dynamism through phosphorylation of cytoskeletal proteins including tau and microtubule-associated protein 2 (MAP-2) (Grant, P., et al. 2001). The phosphorylation sites on tau by GSK3 and CDK5 could account for most of the major phosphorylation sites of fetal tau and paired helical filament tau (PHF) (Imahori, K., et al. 1997; Flaherty, D. B., et al. 2000). CDK5 may catalyze tau hyperphosphorylation by GSK3 in AD brain (Sengupta, A., et al. 1997). Accumulating evidence indicates that deregulation of these kinases is involved in the pathology of neurodegenerative diseases.

Several studies have shown that down-regulation of GSK3 (namely siRNA-mediated α isoform knockout) or inhibition of its activity by lithium chloride (LiCl), an inhibitor of GSK3 (Stambolic, V., et al. 1996; Phiel, C. J., et al. 2001), can significant lower the γ-secretase cleavage of APP (and hence Aβ generation) without affecting Notch cleavage (Phiel, C. J., et al. 2003) through a mechanism yet to be identified. LiCl also reduces tau phosphorylation in rat cultured neurons (Munoz-Montano, J. R., et al. 1997; Hong, M., et al. 1997; Lovestone, S., et al. 1999).

Recently, the pathophysiological functions of APP γCTF (AICD, a γ-secretase cleavage product of APP) have attracted much scrutiny, including modulating transcription of genes, such as GSK-3β, (Kim, H. S., et al. 2003) and APP (von Rotz, R. C., et al. 2004). Several APP-associated proteins such as Fe65, X11α, histone acetyltransferase Tip60, CP2, Shc, Numb and 14-3-3γ have been recently identified to bind to APP CTF/AICD, translocating AICD into nucleus and thus affecting gene expression (Cao, X., et al. 2001; Chang, K. A., et al. 2005; Muresan, Z., et al. 2005; Sumioka, A., et al. 2005; Telese, F., et al. 2005). It has recently been reported that, phosphorylation at Thr668 of AICD, presumably by GSK3 enhances the formation of a ternary complex with Fe65 and CP2 transcription factor and contributes to resultant neurotoxic effects (Kim, H. S., et al. 2003; Chang, K. A., et al. 2006). Furthermore, AICD has been shown to up-regulate the expression of GSK3 in cultured cells (Kim, H. S., et al. 2003) and to activate GSK3 in vivo (Ryan, K. A., et al. 2005). Besides AICD, Aβ has been known for many years to stimulate GSK3 activity (Takashima, A., et al. 2006). Taken together, there appears to be an interplay between Aβ toxicity, AICD and GSK3 activation.

However, a need still exists in the art for more effective treatments for neurodegenerative diseases, such as Alzheimer's disease, neoplastic diseases, and metabolic diseases, such as diabetes.

### BRIEF SUMMARY

In accordance with the purpose of this invention, as embodied and broadly described herein, disclosed are compositions and methods relating to peptides. The peptides can inhibit amyloid beta (Aβ) generation and reduce GSK-3 activities. Further provided are compositions and methods for treating or preventing, for example, neurodegenerative disease, cancer, or diabetes.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows a schematic representation of β-amyloid precursor protein and its cleavages by α-, β-, and γ-secretases.

FIG. 2 shows gene screening using RHKO cell lines.

FIG. 3 shows accumulation of APP βCTF on the cell surface in RHKO cells.

FIG. 4 shows isolation of cell population with enhanced detection of cell surface APP β-CTF.

FIG. 5 shows Western blotting analysis of selected RHKO cell lines.

FIG. 6 shows FG01 was overexpressed in the RHKO cells.

FIG. 7 shows FG01 is predicted as a transmembrane protein with a single transmembrane domain near the C-terminus (FIG. 7A). FIG. 7 further shows tissue distribution of FG01 (FIG. 7B).

FIG. 8 shows FG01 affects Aβ generation.

FIG. 9 shows FG01 regulates GSK-3 activities but not γ-secretase activity for Notch cleavage.

FIG. 10 shows FG01 affects tau phosphorylation.

FIG. 11 shows genomic localization of FG01.

FIG. 12 shows FG01 is a type I transmembrane protein.

FIG. 13 shows expression of FG01 decreases the levels of Aβ and affects APP processing in human cells.

FIG. 14 shows overexpression of FG01 decreases GSK-3 activities and increases the level of β-catenin in mouse N2a cells.

FIG. 15 shows expression of FG01 decreases GSK-3 activities and increases the level of β-catenin in human HEK293 cells.

FIG. 16 shows overexpression of FG01 did not further decrease Aβ levels in LiCl-pretreated N2a cells.

FIG. 17 shows APP/AICD is not required for FG01's regulatory effects on GSK-3 activities.

FIG. 18 shows expression of FG01 suppressed the effects of overexpression of GSK-3β on Aβ in HeLa/swe cells.

FIG. 19 shows expression of FG01 did not change the levels of CDK5 in mouse and human cells.

FIG. 20 shows PKC activity was increased by overexpression of FG01.

FIG. 21 shows FG01 stimulates APP trafficking to cell surface.

FIG. 22 shows FG01 affects Aβ generation through regulating PKA activity.

FIG. 23 shows FG01 interacts with adenylyl cyclase (AC) and regulates the levels of cyclic AMP (cAMP).

FIG. 24 shows the carboxy-terminal region of FG01 is critical for FG01's effects on the activities of PKA and GSK-3.

FIG. 25 shows a comparison of the fg01 gene sequence (SEQ ID NO:23) with the mouse RPS23 gene sequence (SEQ ID NO:24), which indicates that the fg01 gene originated through retroposition of the mouse RPS23 mRNA. Sequence alignment of the reverse and complementary (r/c) sequence of mouse RPS23 (mRPS23) mRNA with fg01. Small letters indicate intron sequence (underlined) or untranslated regions of the exon (no underlined). Capital letters indicate protein-encoding sequence. Additional gene parts of fg01 were recruited from integrated chromosomal sites (not shown). *: nonconserved nucleotide residues.

FIGS. 26A-26D show genetic screening using random homozygous gene knockout (RHKO). (A) The new RHKO gene search vector has a tetracycline-regulated element (TRE) regulated CMV promoter, which drives expression of the puromycin N-acetyl-tranferase gene (pac), a plasmid replication origin and a chloramphenicol resistance marker (Ori-CAT), and a LoxP site in both the 5′LTR (not shown) and the 3′LTR. In addition, there is a Cre recombinase gene (Cre) between the 5′LTR and the 3′LTR. (B) The initial provirus randomly inserted into chromosomes of mammalian cells upon retroviral infection. (C) The final integrated provirus after the expression of the Cre recombinase in the initial provirus, which mediates DNA recombination at the loxP sites. (D) Strategy for screening for Aβ-reducing genes in N2aSwe cells with RHKO vector integration.

FIGS. 27A-27D shows identification of the FG01 cell clone. (A) RHKO libraries of N2aSwe cells were live-immunostained for cell surface βCTF using an Aβ N-terminal specific antibody (FCA18) (Barelli et al., 1997) and screened by multiple rounds of FACS sorting. Less than 0.01% of cells showing βCTF accumulation after first round of FACS sorting were enriched up to 75% following another two rounds of FACS sorting. Y axis, cell number; X axis, fluorescence intensity. (B) Parental N2aSwe cells (I and III) and one cell clone derived from FACS sorting, FG01 (II and IV), were live-immunostained to visualize surface APP βCTF (I and II). Cells were also double immunostained with FITC-VVA (Vicia Vilosa Agglutinin, Vector Laboratories) to stain total surface glycoproteins (III and IV). (C) Parental N2aSwe and FG01 cells were treated with or without 2 μg/ml doxycycline (DOX) for 72 hrs. Equal amounts of cell lysates were subjected to SDS-PAGE and Western blot to detect full length APP and βCTFs. Secreted Aβ was immunoprecipitated from conditioned media and analyzed by Western blot. (D) Parental N2aSwe and FG01 cells were treated with 2 μg/ml doxycycline (+) or DMSO (−) for 72 hrs before RNA was isolated for real-time reverse transcription-PCR to quantify fg01 expression. The level of fg01 in N2aSwe cells treated with DMSO was used as normalization controls (set as one arbitrary unit). *P<0.05. P values were calculated using two-tailed Student's t-test (n=3). Error bars, SEM.

FIGS. 28A-28G show that FG01 is a Type III transmembrane protein. (A) Scheme of the FG01 construct (not drawn to proportion), with a Myc tag at the N-terminus and a His₆ tag at the C-terminus. FG01 has a predicted single trans-membrane domain (TM) near its C-terminus. (B) The Myc-FG01-His₆ vector or a pcDNA control was transiently transfected into N2a cells. Cell lysates were subjected to Western blot (WB) with antibodies against Myc or His₆. (C) FG01, APP and SMAD3 plasmids (all Myc-tagged) were individually transfected into N2a cells. After fractionation of membrane and cytosol, equal volumes of samples from both fractions were subjected to SDS-PAGE and Western analysis with a Myc antibody. *: non-specific band. (D) After FG01 transfection, N2a cells were biotinylated and biotin-labeled membrane proteins were affinity precipitated (AP) with streptivadin and immunoblotted with a Myc antibody. (E) After transfection with the Myc-FG01-His₆ construct, N2a cells were either live-immunostained or permeabilized and immunostained with Myc or His₆ antibody. Cells were then fixed, permeabilized, incubated with Alexa Fluor 488-conjugated secondary antibody and DAPI, and examined by immunofluorescence microscopy. Arrows indicate membrane staining of FG01 in live cells. (F) Equal protein lysates from mouse cortex and hippocampus (hippo) were incubated with an FG01 antibody. After immunoprecipitation, samples were subjected to SDS-PAGE and Western analyzed with the FG01 antibody. (G) An anti-sense probe of fg01 and the corresponding sense probe (as control) were used for in situ hybridization in brain sections from a two-month-old C57B16 mouse. Arrows indicate fg01 expression.

FIGS. 29A-29F show that FG01 inhibits Aβ generation, GSK-3 activity and tau phosphorylation. (A) FG01 or control vector (Con) were transfected into mouse N2aSwe or human HeLaSwe cells. Aβ in conditioned media (secreted or extracellular) and cell lysates (intracellular) was immunoprecipitated and Western blotted with the Aβ antibody 6E10. sAPPα in conditioned media was immunoblotted with 6E10. Cell lysates were immunoblotted with 6E10 for APP/βCTF and with Myc antibody for FG01. (B) ELISA quantification of Aβ40 and Aβ42 levels in conditioned media and lysates of HelaSwe cells with FG01 overexpression. Results were normalized to that of Aβ40 in conditioned media (set as 100). Aβ42 in cell lysates were below detection level and not shown. (C) After transfection with FG01, N2aSwe and HeLaSwe cell lysates were immunoblotted with antibodies against phosphorylated GSK-3α/β, total GSK3/β, and Myc. (D) GSK-3α and GSK-3β in lysates from N2aSwe cells transfected with control or FG01 cDNA were immunoprecipitated with respective antibodies and assayed for in vitro activity. Results were normalized to those of controls (set as one arbitrary unit). (E) N2aSwe cells were first transfected with FG01 or control vector (Con). After equal splitting, cells were treated with 5 mM LiCl or NaCl (as control) for 4 hrs before collection. Conditioned media were assayed for Aβ. Cell lysates were analyzed for total and phosphorylated GSK-3α/β and for FG01. (F) N2a cells were transfected with human tau, equally split, and transfected with FG01 or control vector (Con). The levels of phosphorylated tau including threonine 205 (pT205) and PHF-1, unphopshorylated tau (Tau-1), total tau, and FG01 were analyzed. All error bars indicate SEM. *P<0.05, **P<0.01. P values were calculated using two-tailed Student's t-test (n=3).

FIGS. 30A-30F show that FG01 interacts with adenylate cyclases, upregulates cAMP levels and activates PKA to inhibit GSK-3 activity and Aβ generation. (A) Cells transfected with FG01 or control vector (Con) were analyzed for in vitro PKA activity. Results were normalized to control values (set as one arbitrary unit). (B) After transfection with FG01 or control vectors (Con) and equal splitting, cells were treated with DMSO (control) or the PKA inhibitor H89. Conditioned media were analyzed for Aβ and cell lysates were analyzed for phosphorylated and total CREB, phosphorylated and total GSK-3, and FG01 levels. (C) N2aSwe cells were transfected with fg01-specific RNAi or a scrambled RNAi probe (SC). Total RNA was then extracted and subjected to RT-PCR. The level of fg01 relative to that of β-actin was analyzed and normalized to that from scrambled RNAi probe-transfected cells (set as one arbitrary unit). (D) After RNAi of fg01 expression, conditioned media from N2aSwe cells were analyzed for Aβ, and cell lysates were analyzed for phosphorylated/total CREB and GSK-3. (E) Mouse N2a and rat PC12 cells were transfected with FG01 or control vectors, and cell lysates were assayed for cAMP levels. Data were normalized to control values (as one arbitrary unit). (F) Cells transfected with FG01 or control vectors were lysed in 1% CHAPSO or 1% NP40 buffer. Lysates were incubated with mouse IgG (mIgG), rabbit IgG (rIgG), Myc antibody or adenylate cyclase antibody. Immunoprecipitated proteins were subjected to SDS-PAGE and Western blot analysis with adenylate cyclase or Myc (for FG01) antibodies. All error bars indicate SEM. *P<0.05, **P<0.01. P values were calculated using two-tailed Student's t-test (n=3).

FIGS. 31A-31E show that FG01 overexpression increases PKA activity and synapse number and reduces GSK-3β activity, tau phosphorylation, and Aβ generation in brains of 3×Tg AD mice. (A) Brains from FG01 and littermate controls (Con) on a 3×Tg background at 11 months of age were dissected. One half of the brain was lysed and analyzed for the levels of phosphorylated/total CREB, phosphorylated/total GSK-3, PSD-95, α-tubulin, and phosphorylated (PHF-1) and total tau forms by direct Western blot. Aβ and Myc-FG01 were detected by immunoprecipitation-Western blot using Aβ antibody (6E10) and Myc antibody, respectively. *: non-specific band found specifically in 3×Tg mouse brains. (B) The other half brain from Con (I and III) and FG01 (II and IV) mice was analyzed by immunohistochemistry for Aβ using 6E10 antibody. III and IV are higher magnifications of cortical regions from I and II, respectively. Arrows indicate positive immunoreactivity. (C) Immunohistochemistry for phosphorylated tau (p-tau) using the PHF-1 antibody was analyzed the same as in (B). Arrows indicate positive immunoreactivity. (D) Immunohistochemistry for synapsin was analyzed the same as in (B), except that III and IV are higher magnifications of hippocampal regions from I and II, respectively. (E) Immunostained neurons (>400) in (B) and (C) were counted from 5 randomly selected cortical regions. Ratios of Aβ-positive and phosphorylated tau-positive neurons to total neurons were determined and normalized to those of Con for comparison. *P<0.05. P values were calculated using two-tailed Student's t-test (n=4). Error bars, SEM.

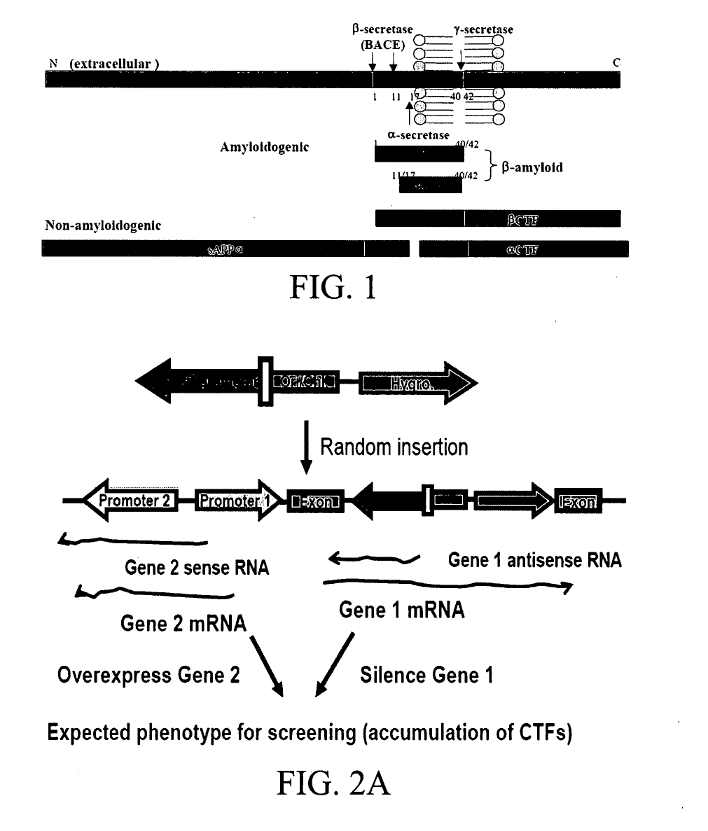

### DETAILED DESCRIPTION

The disclosed method and compositions may be understood more readily by reference to the following detailed description of particular embodiments and the Example included therein and to the Figures and their previous and following description.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a peptide is disclosed and discussed and a number of modifications that can be made to a number of molecules including the peptide are discussed, each and every combination and permutation of peptide and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Those skilled in the art will recognize, or be able to ascertain using no more than routine experimentation, many equivalents to the specific embodiments of the method and compositions described herein. Such equivalents are intended to be encompassed by the following claims.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

A retroposed, reverse-transcribed gene has been discovered that has a major phenotype in ameliorating Alzheimer's disease-like pathology. Retrogenes originate from mRNA-based gene duplication and often evolve novel functions from parental genes, providing important genetic diversity in evolution. So far, a limited number of retrogenes have been discovered and very little is known about their functions, especially those involved in disease pathogenesis. Overproduction of β-amyloid (Aβ) and tau hyperphosphorylation are two major events in the pathogenesis of Alzheimer's disease (AD). Disclosed herein is the identification of a novel gene fg01 whose protein product inhibits both Aβ generation and tau hyperphosphorylation. The molecular mechanism of its function has also been discovered: that is, FG01 protein interacts with adenylate cyclases and increases the level of cyclic AMP which upregulates PKA activity. Activated PKA phosphorylates GSK-3 and reduces its enzymatic activity, leading to the reduced Aβ generation and tau hyperphosphorylation. In vivo evidence confirms that FG01 functions as PKA activator and GSK3 inhibitor, and that these activities are necessary for FG01 to inhibit AD-like pathology (Aβ generation and tau hyperphosphorylation), as well as to increase synaptic markers in a widely used AD transgenic mouse model (3×Tg).

The origination of genes with new functions is an important mechanism for generating genetic novelties during evolution. New genes can originate through different mechanisms, such as exon shuffling, gene duplication, gene fusion/fission, mobile element integration, lateral gene transfer, and retroposition (Long et al., 2003). Retroposition is a process in which a parental mRNA is reverse-transcribed and inserted into the organism's genome, creating duplicate genes in new genomic positions (Hollis et al., 1982; Karin and Richards, 1982; Ueda et al., 1982). Although these intronless retroposed gene copies (retrogenes) were routinely classified as processed pseudogenes because they lack regulatory elements from parental genes (Jeffs and Ashburner, 1991; Mighell et al., 2000; Zhang et al., 2004), emerging evidence shows that occasionally, these retroposed gene copies may recruit regulatory elements as well as protein-encoding sequences near the retroposition site and become expressed and functional (Babushok et al., 2007; Kaessmann et al., 2009; Long et al., 2003; Vinckenbosch et al., 2006). Nevertheless, studies to elucidate the functions of these newly originated genes, especially the functions related to diseases, are limited (Kaessmann et al., 2009; Vinckenbosch et al., 2006).

Alzheimer's disease (AD) is featured by extracellular neuritic plaques, intracellular neurofibrillary tangles (NFTs), synaptic dysfunctions and neural degeneration in vulnerable brain regions (Tanzi and Bertram, 2005). Neuritic plaques are composed of aggregates of heterogeneous β-amyloid (Aβ) peptides, which are derived from β-amyloid precursor protein (APP) through sequential cleavages by β-secretase (BACE1) and the γ-secretase complex (consisting of at least four components: presenilin, nicastrin, APH-1 and PEN-2) (De Strooper, 2003; Zhang and Xu, 2007). Multiple lines of evidence suggest that overproduction/aggregation of Aβ in the brain is a causative factor for AD pathogenesis (Hardy and Selkoe, 2002). NFTs are composed of hyperphosphorylated microtubule associated protein tau (Buee et al., 2000; Lee et al., 2001). Numerous studies have shown that pathogenic APP metabolism/Aβ generation and tau phosphorylation are highly regulated via various signal transduction pathways, e.g., protein kinases and phosphatases (Buxbaum et al., 1994; Fang et al., 2000; Xu et al., 1996) and steroid and peptide hormones (Gasparini et al., 2001; Xu et al., 1998). Among these regulatory pathways, glycogen synthase kinase-3 (GSK-3, α and β isoforms), a serine/threonine kinase essential for a variety of cellular functions including cell adhesion, cell-division, transcription (Frame and Cohen, 2001), has been demonstrated in regulating both Aβ generation and tau phosphorylation (Flaherty et al., 2000; Phiel et al., 2003). This unique feature renders manipulation of GSK-3 activity an attractive therapeutic approach for AD (Frame and Cohen, 2001; Martinez et al., 2002; Medina and Castro, 2008). Hence identification of new genes involved in these processes will be instrumental in developing novel AD therapeutics.

Random Homozygous Knockout (RHKO) is a genome-wide genetic approach that identifies genes based on their biological functions (Li and Cohen, 1996; Liu et al., 2000a; Liu et al., 1999; Liu et al., 2000b). The design of RHKO enables the inactivation of both alleles of randomly addressed chromosomal genes within populations of mammalian cells using gene search vector cassettes that contain a regulated antisense promoter. This strategy has been used successfully to identify genes whose functional homozygous inactivation leads to reversible tumorigenesis, including TSG101, SAM68, Calpain protease nCL-4 and VASP (Li and Cohen, 1996; Liu et al., 2000a; Liu et al., 1999; Liu et al., 2000b).

Using the RHKO approach, a novel gene fg01 was identified that originated through retroposition of the mouse ribosomal protein S23 (RPS23) mRNA. The fg01 gene is reversely transcribed relative to its parental gene, expressing a structurally unrelated, yet functional protein FG01. More importantly, it was demonstrated both in vitro and in vivo that overexpression of the FG01 protein inhibits Aβ generation and tau phosphorylation by inhibiting GSK-3 activity via the adenylate cyclase/protein kinase A (PKA) pathway.

AD is unarguably one of the most important topics of medical research today and so far there is no cure or meaningfully effective treatment for this devastating neurodegenerative disorder. Significant efforts for treating AD have been directed at targeting either Aβ generation or tau phosphorylation, but with little success so far. The disclosed compositions and methods, which are built on the identification of the gene fg01 and the molecular pathway/mechanism by which its product concomitantly reduces Aβ generation and tau phosphorylation, can be used, for example, to treat AD, to analyze the state and progression of AD, and to analyze the effectiveness of AD treatments. Moreover, since components of the FG01-associated pathways, namely PKA and GSK-3, are crucial in physiological processes such as memory/cognition and development and in multiple diseases such as bipolar disorder, diabetes, and cancers, the disclosed compositions and methods can be, for example, used to treat, diagnose, and monitor these and other diseases in which FG01-associated pathways are involved.

### A. COMPOSITIONS

### 1. FG01

i. FG01 Peptide

Provided herein is a peptide for use in treating neurodegenerative diseases such as Alzheimer's disease. The FG01 peptide can comprise the amino acid sequence SEQ ID NO:2. Thus, the peptide can be encoded by, for example, the nucleic acid sequence SEQ ID NO:1.

Thus, provided is a purified polypeptide, comprising an amino acid sequence at least about 70%, 75%, 80%, 85%, 90%, 92%, 92%, 93%, 94%, 95%, 96%, 97%, 98%, 99%, 100% or at least 70%, 75%, 80%, 85%, 90%, 92%, 92%, 93%, 94%, 95%, 96%, 97%, 98%, 99%, 100% identical to the sequence SEQ ID NO:2, or a fragment thereof at least 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 85, 90, or 100 residues in length.

In some aspects, the polypeptide disclosed herein binds adenylyl cyclase. In some aspects, the polypeptide disclosed herein stimulates adenylate cyclase activity. In some aspects, the polypeptide disclosed herein stimulates protein kinase A activity. In some aspects, the polypeptide disclosed herein decreases GSK3β activity. In some aspects, the polypeptide disclosed herein decreases amyloid beta generation. In some aspects, the polypeptide disclosed herein increases APP. In some aspects, the polypeptide disclosed herein does not affect gamma-secretase cleavage of Notch. Thus, any one or more of these disclosed properties, or any other property disclosed herein, can be used to identify a polypeptide disclosed herein. In some aspects, the disclosed polypeptide directly stimulates adenylate cyclase activity, indirectly stimulates protein kinase A activity, indirectly promotes GSK3β phosphorylation, or a combination.

The amino acid sequence can comprise the sequence SEQ ID NO:2, or a fragment thereof at least 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 85, 90, or 100 residues in length. The amino acid sequence can comprise at least 8 consecutive residues of SEQ ID NO:2.

The provided peptide can comprise the C-terminal amino acid residues of SEQ ID NO:2. Thus, provided is a polypeptide comprising an amino acid sequence at least about 70%, at least about 75%, at least about 80%, at least about 85%, at least about 90%, at least about 95%, at least 70%, at least 75%, at least 80%, at least 85%, at least 90%, or at least 95% identical to amino acids 96 to 141 of SEQ ID NO:2. Thus, provided is a polypeptide comprising an amino acid sequence at least about 70% identical to amino acids 96 to 141, 97 to 141, 98 to 141, 99 to 141, 100 to 141, 101 to 141, 102 to 141, 103 to 141, 104 to 141, 105 to 141, 106 to 141, 107 to 141, 108 to 141, 109 to 141, 110 to 141, 111 to 141, 112 to 141, 113 to 141, 114 to 141, 115 to 141, 116 to 141, 117 to 141, 118 to 141, 119 to 141, 120 to 141 of SEQ ID NO:2.

Thus, provided is a polypeptide comprising an amino acid sequence at least about 70%, at least about 75%, at least about 80%, at least about 85%, at least about 90%, at least about 95%, at least 70%, at least 75%, at least 80%, at least 85%, at least 90%, or at least 95% identical to amino acids 96 to 140, 97 to 140, 98 to 140, 99 to 140, 100 to 140, 101 to 140, 102 to 140, 103 to 140, 104 to 140, 105 to 140, 106 to 140, 107 to 140, 108 to 140, 109 to 140, 110 to 140, 111 to 140, 112 to 140, 113 to 140, 114 to 140, 115 to 140, 116 to 140, 117 to 140, 118 to 140, 119 to 140, 120 to 140 of SEQ ID NO:2.

Thus, provided is a polypeptide comprising an amino acid sequence at least about 70%, at least about 75%, at least about 80%, at least about 85%, at least about 90%, at least about 95%, at least 70%, at least 75%, at least 80%, at least 85%, at least 90%, or at least 95% identical to amino acids 96 to 139, 97 to 139, 98 to 139, 99 to 139, 100 to 139, 101 to 139, 102 to 139, 103 to 139, 104 to 139, 105 to 139, 106 to 139, 107 to 139, 108 to 139, 109 to 139, 110 to 139, 111 to 139, 112 to 139, 113 to 139, 114 to 139, 115 to 139, 116 to 139, 117 to 139, 118 to 139, 119 to 139, 120 to 139 of SEQ ID NO:2.

Thus, provided is a polypeptide comprising an amino acid sequence at least about 70%, at least about 75%, at least about 80%, at least about 85%, at least about 90%, at least about 95%, at least 70%, at least 75%, at least 80%, at least 85%, at least 90%, or at least 95% identical to amino acids 96 to 138, 97 to 138, 98 to 138, 99 to 138, 100 to 138, 101 to 138, 102 to 138, 103 to 138, 104 to 138, 105 to 138, 106 to 138, 107 to 138, 108 to 138, 109 to 138, 110 to 138, 111 to 138, 112 to 138, 113 to 138, 114 to 138, 115 to 138, 116 to 138, 117 to 138, 118 to 138, 119 to 138, 120 to 138 of SEQ ID NO:2.

Thus, provided is a polypeptide comprising an amino acid sequence at least about 70%, at least about 75%, at least about 80%, at least about 85%, at least about 90%, at least about 95%, at least 70%, at least 75%, at least 80%, at least 85%, at least 90%, or at least 95% identical to amino acids 96 to 137, 97 to 137, 98 to 137, 99 to 137, 100 to 137, 101 to 137, 102 to 137, 103 to 137, 104 to 137, 105 to 137, 106 to 137, 107 to 137, 108 to 137, 109 to 137, 110 to 137, 111 to 137, 112 to 137, 113 to 137, 114 to 137, 115 to 137, 116 to 137, 117 to 137, 118 to 137, 119 to 137, 120 to 137 of SEQ ID NO:2.

Thus, provided is a polypeptide comprising an amino acid sequence at least about 70%, at least about 75%, at least about 80%, at least about 85%, at least about 90%, at least about 95%, at least 70%, at least 75%, at least 80%, at least 85%, at least 90%, or at least 95% identical to amino acids 96 to 136, 97 to 136, 98 to 136, 99 to 136, 100 to 136, 101 to 136, 102 to 136, 103 to 136, 104 to 136, 105 to 136, 106 to 136, 107 to 136, 108 to 136, 109 to 136, 110 to 136, 111 to 136, 112 to 136, 113 to 136, 114 to 136, 115 to 136, 116 to 136, 117 to 136, 118 to 136, 119 to 136, 120 to 136 of SEQ ID NO:2.

Thus, provided is a polypeptide comprising an amino acid sequence at least about 70%, at least about 75%, at least about 80%, at least about 85%, at least about 90%, at least about 95%, at least 70%, at least 75%, at least 80%, at least 85%, at least 90%, or at least 95% identical to amino acids 96 to 135, 97 to 135, 98 to 135, 99 to 135, 100 to 135, 101 to 135, 102 to 135, 103 to 135, 104 to 135, 105 to 135, 106 to 135, 107 to 135, 108 to 135, 109 to 135, 110 to 135, 111 to 135, 112 to 135, 113 to 135, 114 to 135, 115 to 135, 116 to 135, 117 to 135, 118 to 135, 119 to 135, 120 to 135 of SEQ ID NO:2.

Thus, provided is a polypeptide comprising an amino acid sequence at least about 70%, at least about 75%, at least about 80%, at least about 85%, at least about 90%, at least about 95%, at least 70%, at least 75%, at least 80%, at least 85%, at least 90%, or at least 95% identical to amino acids 96 to 134, 97 to 134, 98 to 134, 99 to 134, 100 to 134, 101 to 134, 102 to 134, 103 to 134, 104 to 134, 105 to 134, 106 to 134, 107 to 134, 108 to 134, 109 to 134, 110 to 134, 111 to 134, 112 to 134, 113 to 134, 114 to 134, 115 to 134, 116 to 134, 117 to 134, 118 to 134, 119 to 134, 120 to 134 of SEQ ID NO:2.

Thus, provided is a polypeptide comprising an amino acid sequence at least about 70%, at least about 75%, at least about 80%, at least about 85%, at least about 90%, at least about 95%, at least 70%, at least 75%, at least 80%, at least 85%, at least 90%, or at least 95% identical to amino acids 96 to 133, 97 to 133, 98 to 133, 99 to 133, 100 to 133, 101 to 133, 102 to 133, 103 to 133, 104 to 133, 105 to 133, 106 to 133, 107 to 133, 108 to 133, 109 to 133, 110 to 133, 111 to 133, 112 to 133, 113 to 133, 114 to 133, 115 to 133, 116 to 133, 117 to 133, 118 to 133, 119 to 133, 120 to 133 of SEQ ID NO:2.

Thus, provided is a polypeptide comprising an amino acid sequence at least about 70%, at least about 75%, at least about 80%, at least about 85%, at least about 90%, at least about 95%, at least 70%, at least 75%, at least 80%, at least 85%, at least 90%, or at least 95% identical to amino acids 96 to 132, 97 to 132, 98 to 132, 99 to 132, 100 to 132, 101 to 132, 102 to 132, 103 to 132, 104 to 132, 105 to 132, 106 to 132, 107 to 132, 108 to 132, 109 to 132, 110 to 132, 111 to 132, 112 to 132, 113 to 132, 114 to 132, 115 to 132, 116 to 132, 117 to 132, 118 to 132, 119 to 132, 120 to 132 of SEQ ID NO:2.

Thus, provided is a polypeptide comprising an amino acid sequence at least about 70%, at least about 75%, at least about 80%, at least about 85%, at least about 90%, at least about 95%, at least 70%, at least 75%, at least 80%, at least 85%, at least 90%, or at least 95% identical to amino acids 96 to 131, 97 to 131, 98 to 131, 99 to 131, 100 to 131, 101 to 131, 102 to 131, 103 to 131, 104 to 131, 105 to 131, 106 to 131, 107 to 131, 108 to 131, 109 to 131, 110 to 131, 111 to 131, 112 to 131, 113 to 131, 114 to 131, 115 to 131, 116 to 131, 117 to 131, 118 to 131, 119 to 131, 120 to 131 of SEQ ID NO:2.

Thus, provided is a polypeptide comprising an amino acid sequence at least about 70%, at least about 75%, at least about 80%, at least about 85%, at least about 90%, at least about 95%, at least 70%, at least 75%, at least 80%, at least 85%, at least 90%, or at least 95% identical to amino acids 96 to 130, 97 to 130, 98 to 130, 99 to 130, 100 to 130, 101 to 130, 102 to 130, 103 to 130, 104 to 130, 105 to 130, 106 to 130, 107 to 130, 108 to 130, 109 to 130, 110 to 130, 111 to 130, 112 to 130, 113 to 130, 114 to 130, 115 to 130, 116 to 130, 117 to 130, 118 to 130, 119 to 130, 120 to 130 of SEQ ID NO:2.

Thus, provided is a polypeptide comprising an amino acid sequence at least about 70%, at least about 75%, at least about 80%, at least about 85%, at least about 90%, at least about 95%, at least 70%, at least 75%, at least 80%, at least 85%, at least 90%, or at least 95% identical to amino acids 96 to 129, 97 to 129, 98 to 129, 99 to 129, 100 to 129, 101 to 129, 102 to 129, 103 to 129, 104 to 129, 105 to 129, 106 to 129, 107 to 129, 108 to 129, 109 to 129, 110 to 129, 111 to 129, 112 to 129, 113 to 129, 114 to 129, 115 to 129, 116 to 129, 117 to 129, 118 to 129, 119 to 129, 120 to 129 of SEQ ID NO:2.

Thus, provided is a polypeptide comprising an amino acid sequence at least about 70%, at least about 75%, at least about 80%, at least about 85%, at least about 90%, at least about 95%, at least 70%, at least 75%, at least 80%, at least 85%, at least 90%, or at least 95% identical to amino acids 96 to 128, 97 to 128, 98 to 128, 99 to 128, 100 to 128, 101 to 128, 102 to 128, 103 to 128, 104 to 128, 105 to 128, 106 to 128, 107 to 128, 108 to 128, 109 to 128, 110 to 128, 111 to 128, 112 to 128, 113 to 128, 114 to 128, 115 to 128, 116 to 128, 117 to 128, 118 to 128, 119 to 128, 120 to 128 of SEQ ID NO:2.

Thus, provided is a polypeptide comprising an amino acid sequence at least about 70%, at least about 75%, at least about 80%, at least about 85%, at least about 90%, at least about 95%, at least 70%, at least 75%, at least 80%, at least 85%, at least 90%, or at least 95% identical to amino acids 96 to 127, 97 to 127, 98 to 127, 99 to 127, 100 to 127, 101 to 127, 102 to 127, 103 to 127, 104 to 127, 105 to 127, 106 to 127, 107 to 127, 108 to 127, 109 to 127, 110 to 127, 111 to 127, 112 to 127, 113 to 127, 114 to 127, 115 to 127, 116 to 127, 117 to 127, 118 to 127, 119 to 127, 120 to 127 of SEQ ID NO:2.

Thus, provided is a polypeptide comprising an amino acid sequence at least about 70%, at least about 75%, at least about 80%, at least about 85%, at least about 90%, at least about 95%, at least 70%, at least 75%, at least 80%, at least 85%, at least 90%, or at least 95% identical to amino acids 96 to 126, 97 to 126, 98 to 126, 99 to 126, 100 to 126, 101 to 126, 102 to 126, 103 to 126, 104 to 126, 105 to 126, 106 to 126, 107 to 126, 108 to 126, 109 to 126, 110 to 126, 111 to 126, 112 to 126, 113 to 126, 114 to 126, 115 to 126, 116 to 126, 117 to 126, 118 to 126, 119 to 126, 120 to 126 of SEQ ID NO:2.

Thus, provided is a polypeptide comprising an amino acid sequence at least about 70%, at least about 75%, at least about 80%, at least about 85%, at least about 90%, at least about 95%, at least 70%, at least 75%, at least 80%, at least 85%, at least 90%, or at least 95% identical to amino acids 96 to 125, 97 to 125, 98 to 125, 99 to 125, 100 to 125, 101 to 125, 102 to 125, 103 to 125, 104 to 125, 105 to 125, 106 to 125, 107 to 125, 108 to 125, 109 to 125, 110 to 125, 111 to 125, 112 to 125, 113 to 125, 114 to 125, 115 to 125, 116 to 125, 117 to 125, 118 to 125, 119 to 125, 120 to 125 of SEQ ID NO:2.

a. Dominant Active

Also provided are functional dominate forms of an FG01 peptide disclosed herein. For example, disclosed is an expression vector comprising a nucleic acid encoding an FG01 peptide disclosed herein and further comprising a nucleic acid, wherein the nucleic acid or a peptide encoded thereby enhances an activity of the FG01 peptide.

Also disclosed is an FG01 peptide comprising amino acid substitutions, deletions, and/or additions that enhances an activity of the FG01 peptide.

“Activities” of a nucleic acid/protein include, for example, transcription, translation, intracellular translocation, secretion, phosphorylation by kinases, cleavage by proteases, homophilic and heterophilic binding to other proteins, ubiquitination.

Thus, the disclosed nucleic acid, peptide, or modification can enhance the stability of FG01 transcript or peptide. The disclosed nucleic acid, peptide, or fusion protein can enhance the binding of FG01 to target proteins.

b. Fusion Protein

Also provided is a fusion protein comprising peptide for use in treating neurodegenerative diseases such as Alzheimer's disease comprising an amino acid sequence at least about 70%, 75%, 80%, 85%, 90%, 92%, 92%, 93%, 94%, 95%, 96%, 97%, 98%, 99%, 100% or at least 70%, 75%, 80%, 85%, 90%, 92%, 92%, 93%, 94%, 95%, 96%, 97%, 98%, 99%, 100% identical to the sequence SEQ ID NO:2, or a fragment thereof at least 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 85, 90, or 100 residues in length, and further comprising an amino acid sequence that binds GSK-3, adenylyl cyclase, or PKA.

Also provided is a fusion protein comprising peptide for use in treating neurodegenerative diseases such as Alzheimer's disease comprising an amino acid sequence at least about 70%, 75%, 80%, 85%, 90%, 92%, 92%, 93%, 94%, 95%, 96%, 97%, 98%, 99%, 100% or at least 70%, 75%, 80%, 85%, 90%, 92%, 92%, 93%, 94%, 95%, 96%, 97%, 98%, 99%, 100% identical to the sequence SEQ ID NO:2, or a fragment thereof at least 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 85, 90, or 100 residues in length, and further comprising a GSK-3 amino acid sequence.

Also provided is a fusion protein comprising peptide for use in treating neurodegenerative diseases such as Alzheimer's disease comprising an amino acid sequence at least about 70%, 75%, 80%, 85%, 90%, 92%, 92%, 93%, 94%, 95%, 96%, 97%, 98%, 99%, 100% or at least 70%, 75%, 80%, 85%, 90%, 92%, 92%, 93%, 94%, 95%, 96%, 97%, 98%, 99%, 100% identical to the sequence SEQ ID NO:2, or a fragment thereof at least 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 85, 90, or 100 residues in length, and further comprising a PKA amino acid sequence.

Fusion proteins, also know as chimeric proteins, are proteins created through the joining of two or more genes which originally coded for separate proteins. Translation of this fusion gene results in a single polypeptide with function properties derived from each of the original proteins. Recombinant fusion proteins can be created artificially by recombinant DNA technology for use in biological research or therapeutics. Chimeric mutant proteins occur naturally when a large-scale mutation, typically a chromosomal translocation, creates a novel coding sequence containing parts of the coding sequences from two different genes.

The functionality of fusion proteins is made possible by the fact that many protein functional domains are modular. In other words, the linear portion of a polypeptide which corresponds to a given domain, such as a tyrosine kinase domain, may be removed from the rest of the protein without destroying its intrinsic enzymatic capability. Thus, any of the herein disclosed functional domains can be used to design a fusion protein.

A recombinant fusion protein is a protein created through genetic engineering of a fusion gene. This typically involves removing the stop codon from a cDNA sequence coding for the first protein, then appending the cDNA sequence of the second protein in frame through ligation or overlap extension PCR. That DNA sequence will then be expressed by a cell as a single protein. The protein can be engineered to include the full sequence of both original proteins, or only a portion of either.

If the two entities are proteins, often linker (or “spacer”) peptides are also added which make it more likely that the proteins fold independently and behave as expected. Especially in the case where the linkers enable protein purification, linkers in protein or peptide fusions are sometimes engineered with cleavage sites for proteases or chemical agents which enable the liberation of the two separate proteins. This technique is often used for identification and purification of proteins, by fusing a GST protein, FLAG peptide, or a hexa-his peptide (aka: a 6xhis-tag) which can be isolated using nickel or cobalt resins (affinity chromatography). Chimeric proteins can also be manufactured with toxins or anti-bodies attached to them in order to study disease development.

Alternatively, internal ribosome entry sites (IRES) elements can be used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (U.S. Pat. Nos. 5,925,565 and 5,935,819; PCT/US99/05781). IRES sequences are known in the art and include those from encephalomycarditis virus (EMCV) (Ghattas, I. R. et al., Mol. Cell. Biol., 11:5848-5849 (1991); BiP protein (Macejak and Sarnow, Nature, 353:91 (1991)); the Antennapedia gene of drosophilia (exons d and e) [Oh et al., Genes & Development, 6:1643-1653 (1992)); those in polio virus [Pelletier and Sonenberg, Nature, 334:320325 (1988); see also Mountford and Smith, TIG, 11:179-184 (1985)).

c. Internalization Sequences

The provided polypeptide can further constitute a fusion protein or otherwise have additional N-terminal, C-terminal, or intermediate amino acid sequences, e.g., linkers or tags. “Linker”, as used herein, is an amino acid sequences or insertion that can be used to connect or separate two distinct polypeptides or polypeptide fragments, wherein the linker does not otherwise contribute to the essential function of the composition. A polypeptide provided herein, can have an amino acid linker comprising, for example, the amino acids GLS, ALS, or LLA. A “tag”, as used herein, refers to a distinct amino acid sequence that can be used to detect or purify the provided polypeptide, wherein the tag does not otherwise contribute to the essential function of the composition. The provided polypeptide can further have deleted N-terminal, C-terminal or intermediate amino acids that do not contribute to the essential activity of the polypeptide.

The disclosed composition can be linked to an internalization sequence or a protein transduction domain to effectively enter the cell. Recent studies have identified several cell penetrating peptides, including the TAT transactivation domain of the HIV virus, antennapedia, and transportan that can readily transport molecules and small peptides across the plasma membrane (Schwarze et al., 1999; Derossi et al., 1996; Yuan et al., 2002). More recently, polyarginine has shown an even greater efficiency of transporting peptides and proteins across the plasma, membrane making it an attractive tool for peptide mediated transport (Fuchs and Raines, 2004). Nonaarginine (R₉, SEQ ID NO:3) has been described as one of the most efficient polyarginine based protein transduction domains, with maximal uptake of significantly greater than TAT or antennapeadia. Peptide mediated cytotoxicity has also been shown to be less with polyarginine-based internalization sequences. R₉ mediated membrane transport is facilitated through heparan sulfate proteoglycan binding and endocytic packaging. Once internalized, heparan is degraded by heparanases, releasing R₉ which leaks into the cytoplasm (Deshayes et al., 2005). Studies have recently shown that derivatives of polyarginine can deliver a full length p53 protein to oral cancer cells, suppressing their growth and metastasis, defining polyarginine as a potent cell penetrating peptide (Takenobu et al., 2002).

Thus, the provided polypeptide can comprise a cellular internalization transporter or sequence. The cellular internalization sequence can be any internalization sequence known or newly discovered in the art, or conservative variants thereof. Non-limiting examples of cellular internalization transporters and sequences include Polyarginine (e.g., R₉), Antennapedia sequences, TAT, HIV-Tat, Penetratin, Antp-3A (Antp mutant), Buforin II, Transportan, MAP (model amphipathic peptide), K-FGF, Ku70, Prion, pVEC, Pep-1, SynB1, Pep-7, HN-1, BGSC (Bis-Guanidinium-Spermidine-Cholesterol, and BGTC (Bis-Guanidinium-Tren-Cholesterol) (see Table 3).

TABLE 3
Cell Internalization Transporters

| Name | Sequence | SEQ ID NO |
|---|---|---|
| Polyarginine | RRRRRRRRR | SEQ ID NO:3 |
| Antp | RQPKJWFPNRRKPWKK | SEQ ID NO:4 |
| HIV-Tat | GRKKRRQRPPQ | SEQ ID NO:5 |
| Penetratin | RQIKIWFQNRRMKWKK | SEQ ID NO:6 |
| Antp-3A | RQIAIWFQNRRMKWAA | SEQ ID NO:7 |
| Tat | RKKRRQRRR | SEQ ID NO:8 |
| Buforin II | TRSSRAGLQFPVGRVHRLLRK | SEQ ID NO:9 |
| Transportan | GWTLNSAGYLLGKINKALAALA | SEQ ID NO:10 |
| | KKIL | |
| model amphipathic | KLALKLALKALKAALKLA | SEQ ID NO:11 |
| peptide (MAP) | | |
| K-FGF | AAVALLPAVLLALLAP | SEQ ID NO:12 |
| Ku70 | VPMLK- PMLKE | SEQ ID NO:13 |
| Prion | MANLGYWLLALFVTMWTDVGL | SEQ ID NO:14 |
| | CKKRPKP | |
| pVEC | LLIILRRRIRKQAHAHSK | SEQ ID NO:15 |
| Pep-1 | KETWWETWWTEWSQPKKKRKV | SEQ ID NO:16 |
| SynB1 | RGGRLSYSRRRFSTSTGR | SEQ ID NO:17 |
| Pep-7 | SDLWEMMMVSLACQY | SEQ ID NO:18 |
| HN-1 | TSPLNIHNGQKL | SEQ ID NO:19 |
| BGSC (Bis- Guanidinium- Spermidine- Cholesterol) | 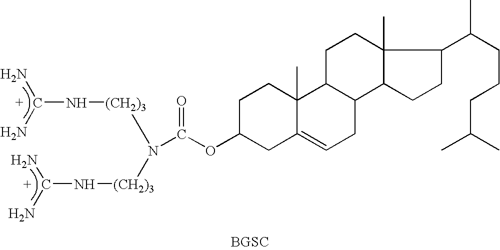 | |
| BGTC (Bis- Guanidinium-Tren- Cholesterol) | 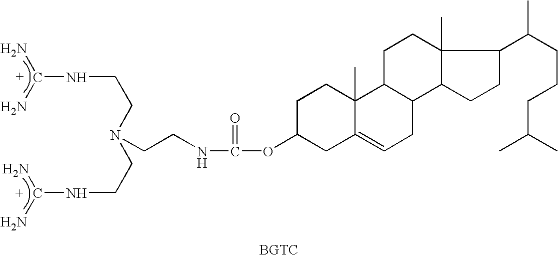 | |

Any other internalization sequences now known or later identified can be combined with any of the disclosed peptides.

d. Protein Variants

Protein variants and derivatives are well understood by those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| | |
|---|---|
| Amino Acid | Abbreviations |

| | | |
|---|---|---|
| Alanine | Ala | A |
| allosoleucine | AIle | |
| Arginine | Arg | R |
| asparagine | Asn | N |
| aspartic acid | Asp | D |
| Cysteine | Cys | C |
| glutamic acid | Glu | E |
| Glutamine | Gln | Q |
| Glycine | Gly | G |
| Histidine | His | H |
| Isolelucine | Ile | I |
| Leucine | Leu | L |
| Lysine | Lys | K |
| phenylalanine | Phe | F |
| proline | Pro | P |
| pyroglutamic acid | pGlu | |
| Serine | Ser | S |
| Threonine | Thr | T |
| Tyrosine | Tyr | Y |
| Tryptophan | Trp | W |
| Valine | Val | V |

TABLE 2
Amino Acid Substitutions
Original Residue Exemplary Conservative
Substitutions, others are known in the art.

| | |
|---|---|
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. For example, SEQ ID NO:2 sets forth a particular sequence of FG01. Specifically disclosed are variants of these and other proteins herein disclosed which have at least, 70% or 75% or 80% or 85% or 90% or 95% homology to the stated sequence. Wherein a sequence is said to have at least about 70% sequence identity, it is understood to also have at least about 75%, 80%, 85%, 90%, 92%, 92%, 93%, 94%, 95%, 96%, 97%, 98%, 99%, or 100% sequence identity.

Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. For example, one of the many nucleic acid sequences that can encode the protein sequence set forth in SEQ ID NO:2 is set forth in SEQ ID NO:1. It is also understood that while no amino acid sequence indicates what particular DNA sequence encodes that protein within an organism, where particular variants of a disclosed protein are disclosed herein, the known nucleic acid sequence that encodes that protein is also known and herein disclosed and described.

It is understood that there are numerous amino acid and peptide analogs which can be incorporated into the disclosed compositions. For example, there are numerous D amino acids or amino acids which have a different functional substituent then the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Engineering Reviews 13:197-216 (1995), Cahill et al., TIBS, 14(10):400-403 (1989); Benner, TIB Tech, 12:158-163 (1994); Ibba and Hennecke, Bio/technology, 12:678-682 (1994) all of which are herein incorporated by reference at least for material related to amino acid analogs).

Molecules can be produced that resemble peptides, but which are not connected via a natural peptide linkage. For example, linkages for amino acids or amino acid analogs can include CH₂NH—, —CH₂S—, —CH₂—CH₂—, —CH═CH—(cis and trans), —COCH₂—, —CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CHH₂—S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

ii. FG01 Nucleic Acid

Also provided is a nucleic acid sequence encoding a polypeptide having the amino acid sequence SEQ ID NO:2, or a fragment thereof at least 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 85, 90, or 100 residues in length. Also provided is an isolated nucleic acid comprising a sequence at least about 70% identical to SEQ ID NO:1, or a fragment thereof at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, or 100 residues in length, wherein the sequence encodes a polypeptide that binds adenylyl cyclase. Thus, the isolated nucleic acid sequence can comprise SEQ ID NO:1, or a fragment thereof at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, or 100 residues in length.

The nucleic acid sequence can comprise the C-terminal-encoding nucleic acid residues of SEQ ID NO:1. Thus, provided is an isolated nucleic acid comprising a sequence at least about 70%, at least about 75%, at least about 80%, at least about 85%, at least about 90%, at least about 95%, at least 70%, at least 75%, at least 80%, at least 85%, at least 90%, or at least 95% identical to nucleic acid residues 288 to 426 of SEQ ID NO:1, or a fragment thereof. Thus, provided is an isolated nucleic acid comprising a sequence at least about 70% identical to nucleic acid residues 288 to 426, 289 to 426, 290 to 426, 291 to 426, 292 to 426, 293 to 426, 294 to 426, 295 to 426, 296 to 426, 297 to 426, 298 to 426, 299 to 426, 300 to 426, 301 to 426, 302 to 426, 303 to 426, 304 to 426, 305 to 426, 306 to 426, 307 to 426, 309 to 426, 310 to 426, 311 to 426, 312 to 426, 313 to 426, 314 to 426, 315 to 426, 316 to 426, 317 to 426, 318 to 426, 319 to 426, 320 to 426, 321 to 426, 322 to 426, 323 to 426, 324 to 426, 325 to 426, 326 to 426, 327 to 426, 328 to 426, 329 to 426, 330 to 426, 331 to 426, 332 to 426, 333 to 426, 334 to 426, 335 to 426, 336 to 426, 337 to 426, 338 to 426, 339 to 426, 340 to 426, 341 to 426, 342 to 426, 343 to 426, 344 to 426, 345 to 426, 346 to 426, 347 to 426, 348 to 426, 349 to 426, 350 to 426, 351 to 426, 352 to 426, 353 to 426, 354 to 426, 355 to 426, 356 to 426, 357 to 426, 358 to 426, 359 to 426, 360 to 426 of SEQ ID NO:1.

Thus, provided is an isolated nucleic acid comprising a sequence at least about 70%, at least about 75%, at least about 80%, at least about 85%, at least about 90%, at least about 95%, at least 70%, at least 75%, at least 80%, at least 85%, at least 90%, or at least 95% identical to nucleic acid residues 288 to 425, 289 to 425, 290 to 425, 291 to 425, 292 to 425, 293 to 425, 294 to 425, 295 to 425, 296 to 425, 297 to 425, 298 to 425, 299 to 425, 300 to 425, 301 to 425, 302 to 425, 303 to 425, 304 to 425, 305 to 425, 306 to 425, 307 to 425, 309 to 425, 310 to 425, 311 to 425, 312 to 425, 313 to 425, 314 to 425, 315 to 425, 316 to 425, 317 to 425, 318 to 425, 319 to 425, 320 to 425, 321 to 425, 322 to 425, 323 to 425, 324 to 425, 325 to 425, 326 to 425, 327 to 425, 328 to 425, 329 to 425, 330 to 425, 331 to 425, 332 to 425, 333 to 425, 334 to 425, 335 to 425, 336 to 425, 337 to 425, 338 to 425, 339 to 425, 340 to 425, 341 to 425, 342 to 425, 343 to 425, 344 to 425, 345 to 425, 346 to 425, 347 to 425, 348 to 425, 349 to 425, 350 to 425, 351 to 425, 352 to 425, 353 to 425, 354 to 425, 355 to 425, 356 to 425, 357 to 425, 358 to 425, 359 to 425, 360 to 425 of SEQ ID NO:1.

Thus, provided is an isolated nucleic acid comprising a sequence at least about 70%, at least about 75%, at least about 80%, at least about 85%, at least about 90%, at least about 95%, at least 70%, at least 75%, at least 80%, at least 85%, at least 90%, or at least 95% identical to nucleic acid residues 288 to 424, 289 to 424, 290 to 424, 291 to 424, 292 to 424, 293 to 424, 294 to 424, 295 to 424, 296 to 424, 297 to 424, 298 to 424, 299 to 424, 300 to 424, 301 to 424, 302 to 424, 303 to 424, 304 to 424, 305 to 424, 306 to 424, 307 to 424, 309 to 424, 310 to 424, 311 to 424, 312 to 424, 313 to 424, 314 to 424, 315 to 424, 316 to 424, 317 to 424, 318 to 424, 319 to 424, 320 to 424, 321 to 424, 322 to 424, 323 to 424, 324 to 424, 325 to 424, 326 to 424, 327 to 424, 328 to 424, 329 to 424, 330 to 424, 331 to 424, 332 to 424, 333 to 424, 334 to 424, 335 to 424, 336 to 424, 337 to 424, 338 to 424, 339 to 424, 340 to 424, 341 to 424, 342 to 424, 343 to 424, 344 to 424, 345 to 424, 346 to 424, 347 to 424, 348 to 424, 349 to 424, 350 to 424, 351 to 424, 352 to 424, 353 to 424, 354 to 424, 355 to 424, 356 to 424, 357 to 424, 358 to 424, 359 to 424, 360 to 424 of SEQ ID NO:1.

Thus, provided is an isolated nucleic acid comprising a sequence at least about 70%, at least about 75%, at least about 80%, at least about 85%, at least about 90%, at least about 95%, at least 70%, at least 75%, at least 80%, at least 85%, at least 90%, or at least 95% identical to nucleic acid residues 288 to 423, 289 to 423, 290 to 423, 291 to 423, 292 to 423, 293 to 423, 294 to 423, 295 to 423, 296 to 423, 297 to 423, 298 to 423, 299 to 423, 300 to 423, 301 to 423, 302 to 423, 303 to 423, 304 to 423, 305 to 423, 306 to 423, 307 to 423, 309 to 423, 310 to 423, 311 to 423, 312 to 423, 313 to 423, 314 to 423, 315 to 423, 316 to 423, 317 to 423, 318 to 423, 319 to 423, 320 to 423, 321 to 423, 322 to 423, 323 to 423, 324 to 423, 325 to 423, 326 to 423, 327 to 423, 328 to 423, 329 to 423, 330 to 423, 331 to 423, 332 to 423, 333 to 423, 334 to 423, 335 to 423, 336 to 423, 337 to 423, 338 to 423, 339 to 423, 340 to 423, 341 to 423, 342 to 423, 343 to 423, 344 to 423, 345 to 423, 346 to 423, 347 to 423, 348 to 423, 349 to 423, 350 to 423, 351 to 423, 352 to 423, 353 to 423, 354 to 423, 355 to 423, 356 to 423, 357 to 423, 358 to 423, 359 to 423, 360 to 423 of SEQ ID NO:1.

Thus, provided is an isolated nucleic acid comprising a sequence at least about 70%, at least about 75%, at least about 80%, at least about 85%, at least about 90%, at least about 95%, at least 70%, at least 75%, at least 80%, at least 85%, at least 90%, or at least 95% identical to nucleic acid residues 288 to 422, 289 to 422, 290 to 422, 291 to 422, 292 to 422, 293 to 422, 294 to 422, 295 to 422, 296 to 422, 297 to 422, 298 to 422, 299 to 422, 300 to 422, 301 to 422, 302 to 422, 303 to 422, 304 to 422, 305 to 422, 306 to 422, 307 to 422, 309 to 422, 310 to 422, 311 to 422, 312 to 422, 313 to 422, 314 to 422, 315 to 422, 316 to 422, 317 to 422, 318 to 422, 319 to 422, 320 to 422, 321 to 422, 322 to 422, 323 to 422, 324 to 422, 325 to 422, 326 to 422, 327 to 422, 328 to 422, 329 to 422, 330 to 422, 331 to 422, 332 to 422, 333 to 422, 334 to 422, 335 to 422, 336 to 422, 337 to 422, 338 to 422, 339 to 422, 340 to 422, 341 to 422, 342 to 422, 343 to 422, 344 to 422, 345 to 422, 346 to 422, 347 to 422, 348 to 422, 349 to 422, 350 to 422, 351 to 422, 352 to 422, 353 to 422, 354 to 422, 355 to 422, 356 to 422, 357 to 422, 358 to 422, 359 to 422, 360 to 422 of SEQ ID NO:1.

Thus, provided is an isolated nucleic acid comprising a sequence at least about 70%, at least about 75%, at least about 80%, at least about 85%, at least about 90%, at least about 95%, at least 70%, at least 75%, at least 80%, at least 85%, at least 90%, or at least 95% identical to nucleic acid residues 288 to 421, 289 to 421, 290 to 421, 291 to 421, 292 to 421, 293 to 421, 294 to 421, 295 to 421, 296 to 421, 297 to 421, 298 to 421, 299 to 421, 300 to 421, 301 to 421, 302 to 421, 303 to 421, 304 to 421, 305 to 421, 306 to 421, 307 to 421, 309 to 421, 310 to 421, 311 to 421, 312 to 421, 313 to 421, 314 to 421, 315 to 421, 316 to 421, 317 to 421, 318 to 421, 319 to 421, 320 to 421, 321 to 421, 322 to 421, 323 to 421, 324 to 421, 325 to 421, 326 to 421, 327 to 421, 328 to 421, 329 to 421, 330 to 421, 331 to 421, 332 to 421, 333 to 421, 334 to 421, 335 to 421, 336 to 421, 337 to 421, 338 to 421, 339 to 421, 340 to 421, 341 to 421, 342 to 421, 343 to 421, 344 to 421, 345 to 421, 346 to 421, 347 to 421, 348 to 421, 349 to 421, 350 to 421, 351 to 421, 352 to 421, 353 to 421, 354 to 421, 355 to 421, 356 to 421, 357 to 421, 358 to 421, 359 to 421, 360 to 421 of SEQ ID NO:1.

Thus, provided is an isolated nucleic acid comprising a sequence at least about 70%, at least about 75%, at least about 80%, at least about 85%, at least about 90%, at least about 95%, at least 70%, at least 75%, at least 80%, at least 85%, at least 90%, or at least 95% identical to nucleic acid residues 288 to 421, 289 to 421, 290 to 421, 291 to 421, 292 to 421, 293 to 421, 294 to 421, 295 to 421, 296 to 421, 297 to 421, 298 to 421, 299 to 421, 300 to 421, 301 to 421, 302 to 421, 303 to 421, 304 to 421, 305 to 421, 306 to 421, 307 to 421, 309 to 421, 310 to 421, 311 to 421, 312 to 421, 313 to 421, 314 to 421, 315 to 421, 316 to 421, 317 to 421, 318 to 421, 319 to 421, 320 to 421, 321 to 421, 322 to 421, 323 to 421, 324 to 421, 325 to 421, 326 to 421, 327 to 421, 328 to 421, 329 to 421, 330 to 421, 331 to 421, 332 to 421, 333 to 421, 334 to 421, 335 to 421, 336 to 421, 337 to 421, 338 to 421, 339 to 421, 340 to 421, 341 to 421, 342 to 421, 343 to 421, 344 to 421, 345 to 421, 346 to 421, 347 to 421, 348 to 421, 349 to 421, 350 to 421, 351 to 421, 352 to 421, 353 to 421, 354 to 421, 355 to 421, 356 to 421, 357 to 421, 358 to 421, 359 to 421, 360 to 421 of SEQ ID NO:1.

Thus, provided is an isolated nucleic acid comprising a sequence at least about 70%, at least about 75%, at least about 80%, at least about 85%, at least about 90%, at least about 95%, at least 70%, at least 75%, at least 80%, at least 85%, at least 90%, or at least 95% identical to nucleic acid residues 288 to 419, 289 to 419, 290 to 419, 291 to 419, 292 to 419, 293 to 419, 294 to 419, 295 to 419, 296 to 419, 297 to 419, 298 to 419, 299 to 419, 300 to 419, 301 to 419, 302 to 419, 303 to 419, 304 to 419, 305 to 419, 306 to 419, 307 to 419, 309 to 419, 310 to 419, 311 to 419, 312 to 419, 313 to 419, 314 to 419, 315 to 419, 316 to 419, 317 to 419, 318 to 419, 319 to 419, 320 to 419, 321 to 419, 322 to 419, 323 to 419, 324 to 419, 325 to 419, 326 to 419, 327 to 419, 328 to 419, 329 to 419, 330 to 419, 331 to 419, 332 to 419, 333 to 419, 334 to 419, 335 to 419, 336 to 419, 337 to 419, 338 to 419, 339 to 419, 340 to 419, 341 to 419, 342 to 419, 343 to 419, 344 to 419, 345 to 419, 346 to 419, 347 to 419, 348 to 419, 349 to 419, 350 to 419, 351 to 419, 352 to 419, 353 to 419, 354 to 419, 355 to 419, 356 to 419, 357 to 419, 358 to 419, 359 to 419, 360 to 419 of SEQ ID NO:1.

Thus, provided is an isolated nucleic acid comprising a sequence at least about 70%, at least about 75%, at least about 80%, at least about 85%, at least about 90%, at least about 95%, at least 70%, at least 75%, at least 80%, at least 85%, at least 90%, or at least 95% identical to nucleic acid residues 288 to 418, 289 to 418, 290 to 418, 291 to 418, 292 to 418, 293 to 418, 294 to 418, 295 to 418, 296 to 418, 297 to 418, 298 to 418, 299 to 418, 300 to 418, 301 to 418, 302 to 418, 303 to 418, 304 to 418, 305 to 418, 306 to 418, 307 to 418, 309 to 418, 310 to 418, 311 to 418, 312 to 418, 313 to 418, 314 to 418, 315 to 418, 316 to 418, 317 to 418, 318 to 418, 319 to 418, 320 to 418, 321 to 418, 322 to 418, 323 to 418, 324 to 418, 325 to 418, 326 to 418, 327 to 418, 328 to 418, 329 to 418, 330 to 418, 331 to 418, 332 to 418, 333 to 418, 334 to 418, 335 to 418, 336 to 418, 337 to 418, 338 to 418, 339 to 418, 340 to 418, 341 to 418, 342 to 418, 343 to 418, 344 to 418, 345 to 418, 346 to 418, 347 to 418, 348 to 418, 349 to 418, 350 to 418, 351 to 418, 352 to 418, 353 to 418, 354 to 418, 355 to 418, 356 to 418, 357 to 418, 358 to 418, 359 to 418, 360 to 418 of SEQ ID NO:1.

Thus, provided is an isolated nucleic acid comprising a sequence at least about 70%, at least about 75%, at least about 80%, at least about 85%, at least about 90%, at least about 95%, at least 70%, at least 75%, at least 80%, at least 85%, at least 90%, or at least 95% identical to nucleic acid residues 288 to 417, 289 to 417, 290 to 417, 291 to 417, 292 to 417, 293 to 417, 294 to 417, 295 to 417, 296 to 417, 297 to 417, 298 to 417, 299 to 417, 300 to 417, 301 to 417, 302 to 417, 303 to 417, 304 to 417, 305 to 417, 306 to 417, 307 to 417, 309 to 417, 310 to 417, 311 to 417, 312 to 417, 313 to 417, 314 to 417, 315 to 417, 316 to 417, 317 to 417, 318 to 417, 319 to 417, 320 to 417, 321 to 417, 322 to 417, 323 to 417, 324 to 417, 325 to 417, 326 to 417, 327 to 417, 328 to 417, 329 to 417, 330 to 417, 331 to 417, 332 to 417, 333 to 417, 334 to 417, 335 to 417, 336 to 417, 337 to 417, 338 to 417, 339 to 417, 340 to 417, 341 to 417, 342 to 417, 343 to 417, 344 to 417, 345 to 417, 346 to 417, 347 to 417, 348 to 417, 349 to 417, 350 to 417, 351 to 417, 352 to 417, 353 to 417, 354 to 417, 355 to 417, 356 to 417, 357 to 417, 358 to 417, 359 to 417, 360 to 417 of SEQ ID NO:1.

Thus, provided is an isolated nucleic acid comprising a sequence at least about 70%, at least about 75%, at least about 80%, at least about 85%, at least about 90%, at least about 95%, at least 70%, at least 75%, at least 80%, at least 85%, at least 90%, or at least 95% identical to nucleic acid residues 288 to 416, 289 to 416, 290 to 416, 291 to 416, 292 to 416, 293 to 416, 294 to 416, 295 to 416, 296 to 416, 297 to 416, 298 to 416, 299 to 416, 300 to 416, 301 to 416, 302 to 416, 303 to 416, 304 to 416, 305 to 416, 306 to 416, 307 to 416, 309 to 416, 310 to 416, 311 to 416, 312 to 416, 313 to 416, 314 to 416, 315 to 416, 316 to 416, 317 to 416, 318 to 416, 319 to 416, 320 to 416, 321 to 416, 322 to 416, 323 to 416, 324 to 416, 325 to 416, 326 to 416, 327 to 416, 328 to 416, 329 to 416, 330 to 416, 331 to 416, 332 to 416, 333 to 416, 334 to 416, 335 to 416, 336 to 416, 337 to 416, 338 to 416, 339 to 416, 340 to 416, 341 to 416, 342 to 416, 343 to 416, 344 to 416, 345 to 416, 346 to 416, 347 to 416, 348 to 416, 349 to 416, 350 to 416, 351 to 416, 352 to 416, 353 to 416, 354 to 416, 355 to 416, 356 to 416, 357 to 416, 358 to 416, 359 to 416, 360 to 416 of SEQ ID NO:1.

Thus, provided an isolated nucleic acid comprising a sequence at least about 70%, at least about 75%, at least about 80%, at least about 85%, at least about 90%, at least about 95%, at least 70%, at least 75%, at least 80%, at least 85%, at least 90%, or at least 95% identical to nucleic acid residues 288 to 415, 289 to 415, 290 to 415, 291 to 415, 292 to 415, 293 to 415, 294 to 415, 295 to 415, 296 to 415, 297 to 415, 298 to 415, 299 to 415, 300 to 415, 301 to 415, 302 to 415, 303 to 415, 304 to 415, 305 to 415, 306 to 415, 307 to 415, 309 to 415, 310 to 415, 311 to 415, 312 to 415, 313 to 415, 314 to 415, 315 to 415, 316 to 415, 317 to 415, 318 to 415, 319 to 415, 320 to 415, 321 to 415, 322 to 415, 323 to 415, 324 to 415, 325 to 415, 326 to 415, 327 to 415, 328 to 415, 329 to 415, 330 to 415, 331 to 415, 332 to 415, 333 to 415, 334 to 415, 335 to 415, 336 to 415, 337 to 415, 338 to 415, 339 to 415, 340 to 415, 341 to 415, 342 to 415, 343 to 415, 344 to 415, 345 to 415, 346 to 415, 347 to 415, 348 to 415, 349 to 415, 350 to 415, 351 to 415, 352 to 415, 353 to 415, 354 to 415, 355 to 415, 356 to 415, 357 to 415, 358 to 415, 359 to 415, 360 to 415 of SEQ ID NO:1.

Thus, provided is an isolated nucleic acid comprising a sequence at least about 70%, at least about 75%, at least about 80%, at least about 85%, at least about 90%, at least about 95%, at least 70%, at least 75%, at least 80%, at least 85%, at least 90%, or at least 95% identical to nucleic acid residues 288 to 414, 289 to 414, 290 to 414, 291 to 414, 292 to 414, 293 to 414, 294 to 414, 295 to 414, 296 to 414, 297 to 414, 298 to 414, 299 to 414, 300 to 414, 301 to 414, 302 to 414, 303 to 414, 304 to 414, 305 to 414, 306 to 414, 307 to 414, 309 to 414, 310 to 414, 311 to 414, 312 to 414, 313 to 414, 314 to 414, 315 to 414, 316 to 414, 317 to 414, 318 to 414, 319 to 414, 320 to 414, 321 to 414, 322 to 414, 323 to 414, 324 to 414, 325 to 414, 326 to 414, 327 to 414, 328 to 414, 329 to 414, 330 to 414, 331 to 414, 332 to 414, 333 to 414, 334 to 414, 335 to 414, 336 to 414, 337 to 414, 338 to 414, 339 to 414, 340 to 414, 341 to 414, 342 to 414, 343 to 414, 344 to 414, 345 to 414, 346 to 414, 347 to 414, 348 to 414, 349 to 414, 350 to 414, 351 to 414, 352 to 414, 353 to 414, 354 to 414, 355 to 414, 356 to 414, 357 to 414, 358 to 414, 359 to 414, 360 to 414 of SEQ ID NO:1.

Thus, provided is an isolated nucleic acid comprising a sequence at least about 70%, at least about 75%, at least about 80%, at least about 85%, at least about 90%, at least about 95%, at least 70%, at least 75%, at least 80%, at least 85%, at least 90%, or at least 95% identical to nucleic acid residues 288 to 413, 289 to 413, 290 to 413, 291 to 413, 292 to 413, 293 to 413, 294 to 413, 295 to 413, 296 to 413, 297 to 413, 298 to 413, 299 to 413, 300 to 413, 301 to 413, 302 to 413, 303 to 413, 304 to 413, 305 to 413, 306 to 413, 307 to 413, 309 to 413, 310 to 413, 311 to 413, 312 to 413, 313 to 413, 314 to 413, 315 to 413, 316 to 413, 317 to 413, 318 to 413, 319 to 413, 320 to 413, 321 to 413, 322 to 413, 323 to 413, 324 to 413, 325 to 413, 326 to 413, 327 to 413, 328 to 413, 329 to 413, 330 to 413, 331 to 413, 332 to 413, 333 to 413, 334 to 413, 335 to 413, 336 to 413, 337 to 413, 338 to 413, 339 to 413, 340 to 413, 341 to 413, 342 to 413, 343 to 413, 344 to 413, 345 to 413, 346 to 413, 347 to 413, 348 to 413, 349 to 413, 350 to 413, 351 to 413, 352 to 413, 353 to 413, 354 to 413, 355 to 413, 356 to 413, 357 to 413, 358 to 413, 359 to 413, 360 to 413 of SEQ ID NO:1.

Thus, provided is an isolated nucleic acid comprising a sequence at least about 70%, at least about 75%, at least about 80%, at least about 85%, at least about 90%, at least about 95%, at least 70%, at least 75%, at least 80%, at least 85%, at least 90%, or at least 95% identical to nucleic acid residues 288 to 412, 289 to 412, 290 to 412, 291 to 412, 292 to 412, 293 to 412, 294 to 412, 295 to 412, 296 to 412, 297 to 412, 298 to 412, 299 to 412, 300 to 412, 301 to 412, 302 to 412, 303 to 412, 304 to 412, 305 to 412, 306 to 412, 307 to 412, 309 to 412, 310 to 412, 311 to 412, 312 to 412, 313 to 412, 314 to 412, 315 to 412, 316 to 412, 317 to 412, 318 to 412, 319 to 412, 320 to 412, 321 to 412, 322 to 412, 323 to 412, 324 to 412, 325 to 412, 326 to 412, 327 to 412, 328 to 412, 329 to 412, 330 to 412, 331 to 412, 332 to 412, 333 to 412, 334 to 412, 335 to 412, 336 to 412, 337 to 412, 338 to 412, 339 to 412, 340 to 412, 341 to 412, 342 to 412, 343 to 412, 344 to 412, 345 to 412, 346 to 412, 347 to 412, 348 to 412, 349 to 412, 350 to 412, 351 to 412, 352 to 412, 353 to 412, 354 to 412, 355 to 412, 356 to 412, 357 to 412, 358 to 412, 359 to 412, 360 to 412 of SEQ ID NO:1.

Thus, provided is an isolated nucleic acid comprising a sequence at least about 70%, at least about 75%, at least about 80%, at least about 85%, at least about 90%, at least about 95%, at least 70%, at least 75%, at least 80%, at least 85%, at least 90%, or at least 95% identical to nucleic acid residues 288 to 411, 289 to 411, 290 to 411, 291 to 411, 292 to 411, 293 to 411, 294 to 411, 295 to 411, 296 to 411, 297 to 411, 298 to 411, 299 to 411, 300 to 411, 301 to 411, 302 to 411, 303 to 411, 304 to 411, 305 to 411, 306 to 411, 307 to 411, 309 to 411, 310 to 411, 311 to 411, 312 to 411, 313 to 411, 314 to 411, 315 to 411, 316 to 411, 317 to 411, 318 to 411, 319 to 411, 320 to 411, 321 to 411, 322 to 411, 323 to 411, 324 to 411, 325 to 411, 326 to 411, 327 to 411, 328 to 411, 329 to 411, 330 to 411, 331 to 411, 332 to 411, 333 to 411, 334 to 411, 335 to 411, 336 to 411, 337 to 411, 338 to 411, 339 to 411, 340 to 411, 341 to 411, 342 to 411, 343 to 411, 344 to 411, 345 to 411, 346 to 411, 347 to 411, 348 to 411, 349 to 411, 350 to 411, 351 to 411, 352 to 411, 353 to 411, 354 to 411, 355 to 411, 356 to 411, 357 to 411, 358 to 411, 359 to 411, 360 to 411 of SEQ ID NO:1.

Thus, provided is an isolated nucleic acid comprising a sequence at least about 70%, at least about 75%, at least about 80%, at least about 85%, at least about 90%, at least about 95%, at least 70%, at least 75%, at least 80%, at least 85%, at least 90%, or at least 95% identical to nucleic acid residues 288 to 410, 289 to 410, 290 to 410, 291 to 410, 292 to 410, 293 to 410, 294 to 410, 295 to 410, 296 to 410, 297 to 410, 298 to 410, 299 to 410, 300 to 410, 301 to 410, 302 to 410, 303 to 410, 304 to 410, 305 to 410, 306 to 410, 307 to 410, 309 to 410, 310 to 410, 311 to 410, 312 to 410, 313 to 410, 314 to 410, 315 to 410, 316 to 410, 317 to 410, 318 to 410, 319 to 410, 320 to 410, 321 to 410, 322 to 410, 323 to 410, 324 to 410, 325 to 410, 326 to 410, 327 to 410, 328 to 410, 329 to 410, 330 to 410, 331 to 410, 332 to 410, 333 to 410, 334 to 410, 335 to 410, 336 to 410, 337 to 410, 338 to 410, 339 to 410, 340 to 410, 341 to 410, 342 to 410, 343 to 410, 344 to 410, 345 to 410, 346 to 410, 347 to 410, 348 to 410, 349 to 410, 350 to 410, 351 to 410, 352 to 410, 353 to 410, 354 to 410, 355 to 410, 356 to 410, 357 to 410, 358 to 410, 359 to 410, 360 to 410 of SEQ ID NO:1.

Also disclosed is an isolated nucleic acid of at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, or 100 nucleotides that hybridizes under stringent conditions to a hybridization probe consisting of the sequence SEQ ID NO:1 or the complement of SEQ ID NO:1.

Also disclosed herein is an expression vector comprising an isolated nucleic acid disclosed herein operably linked to an expression control sequence. Thus, disclosed herein is an expression vector comprising the nucleic acid sequence SEQ ID NO:1 operably linked to an expression control sequence.

The expression control sequence can be a tissue specific promoter. Any tissues specific promoter can be used. For example, neural, tumor, and pancreatic specific promoters are disclosed. Examples of some tissue-specific promoters include but are not limited to MUC1, EIIA, ACTB, WAP, bHLH-EC2, HOXA-1, Alpha-fetoprotein (AFP), opsin, CR1/2, Fc-γ-Receptor 1 (Fc-γ-R1), MMTVD-LTR, the human insulin promoter, Pdha-2. HOXA-1 is a neuronal tissue specific promoter, and as such, proteins expressed under the control of HOXA-1 are only expressed in neuronal tissue. Sequences for these and other tissue-specific promoters are known in the art and can be found, for example, in Genbank, at www.pubmed.gov.

The expression control sequence can be an inducible promoter. For example, tetracycline controlled transcriptional activation is a method of inducible expression where transcription is reversibly turned on or off in the presence of the antibiotic tetracycline or one of its derivatives (etc. doxycycline). In nature, pTet promotes TetR, the repressor, and TetA, the protein that pumps tetracycline antibiotic out of the cell. Two systems named Tet-off and Tet-on are used.

The Tet-off system makes use of the tetracycline transactivator (tTA) protein created by fusing one protein, TetR (tetracycline repressor), found in Escherichia coli bacteria with another protein, VP16, produced by the Herpes Simplex Virus. The tTA protein binds on DNA at a ‘tet’O operator. Once bound the ‘tet’O operator will activate a promoter coupled to the ‘tet’O operator, activating the transcription of nearby gene. Tetracycline derivatives bind tTA and render it incapable of binding to TRE sequences, therefore preventing transactivation of target genes. This expression system is also used in generation of transgenic mice, which conditionally express gene of interest.

The Tet-on system works in the opposite fashion. In that system the rtTA protein is only capable of binding the operator when bound by doxycycline. Thus the introduction of doxycyline to the system initiates the transcription of the genetic product. The tet-on system is sometimes preferred for the faster responsiveness.

Also disclosed for use in the provided compositions and methods are Cre, FRT and ER (estrogen receptor) conditional gene expression systems. In Cre and FRT systems, activation of knockout of the gene is irreversible once recombination is accomplished, while in Tet and ER systems it is reversible. Tet system has very tight control on expression, while ER system is somewhat leaky. However, Tet system, which depends on transcription and subsequent translation of target gene, is not as fast acting as ER system, which stabilizes the already expressed target protein upon hormone administration.

a. Nucleic Acids

There are a variety of molecules disclosed herein that are nucleic acid based, including for example the nucleic acids that encode, for example FG01 peptide, or fragments thereof, as well as various functional nucleic acids. The disclosed nucleic acids can be made up of for example, nucleotides, nucleotide analogs, or nucleotide substitutes. Non-limiting examples of these and other molecules are discussed herein. It is understood that for example, when a vector is expressed in a cell, the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

(A) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate). There are many varieties of these types of molecules available in the art and available herein.

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties. There are many varieties of these types of molecules available in the art and available herein.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid. There are many varieties of these types of molecules available in the art and available herein.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556). There are many varieties of these types of molecules available in the art and available herein.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

(B) Sequences

There are a variety of sequences related to the protein molecules involved in the signaling pathways disclosed herein, all of which are encoded by nucleic acids or are nucleic acids. The sequences for the human analogs of these genes, as well as other analogs, and alleles of these genes, and splice variants and other types of variants, are available in a variety of protein and gene databases, including Genbank. Those sequences available at the time of filing this application at Genbank are herein incorporated by reference in their entireties as well as for individual subsequences contained therein. Genbank can be accessed at http://www.ncbi.nih.gov/entrez/query.fcgi. Those of skill in the art understand how to resolve sequence discrepancies and differences and to adjust the compositions and methods relating to a particular sequence to other related sequences. Primers and/or probes can be designed for any given sequence given the information disclosed herein and known in the art.

(C) Primers and Probes

Disclosed are compositions including primers and probes, which are capable of interacting with the disclosed nucleic acids, such as the nucleic acid encoding FG01 as disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the disclosed nucleic acids or region of the nucleic acids or they hybridize with the complement of the nucleic acids or complement of a region of the nucleic acids.

The size of the primers or probes for interaction with the nucleic acids in certain embodiments can be any size that supports the desired enzymatic manipulation of the primer, such as DNA amplification or the simple hybridization of the probe or primer. A typical primer or probe would be at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments a primer or probe can be less than or equal to 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In certain embodiments this product is at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments the product is less than or equal to 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

b. Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

(A) Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell. Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell. Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, α-fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

(B) Markers

The viral vectors can include nucleic acid sequence encoding a marker product. This marker product is used to determine if the gene has been delivered to the cell and once delivered is being expressed. Preferred marker genes are the E. Coli lacZ gene, which encodes β-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have an appropriate gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

### 2. Cells

Also disclosed herein is a cultured cell comprising any of the nucleic acids disclosed herein operably linked to an expression control sequence. The cell can be any cell or cell line, including transformed cells and primary cell lines, that can be used to produce recombinant protein. In some aspects, the cell is a eukaryotic cell. For example, the cell can be a Chinese Hamster Ovary (CHO) cell. CHO cells are a cell line derived from Chinese Hamster ovary cells. The cell can be a HEK 293 cell. HEK29 cells were generated by transformation of human embryonic kidney cell cultures (hence HEK) with sheared adenovirus 5 DNA. The cell can be a SF9 cell. SF9 cells are an insect cell line derived from Spodoptera frugiperda much used for production of recombinant protein.

In some aspects, the cell is a stem cell. One category of stem cells is a pluripotent embryonic stem cell. A “pluripotent stem cell” as used herein means a cell which can give rise to many differentiated cell types in an embryo or adult, including the germ cells (sperm and eggs). Pluripotent stem cells are also capable of self-renewal. Thus, these cells not only populate the germ line and give rise to a plurality of terminally differentiated cells which comprise the adult specialized organs, but also are able to regenerate themselves. One category of stem cells are cells which are capable of self renewal and which can differentiate into cell types of the mesoderm, ectoderm, and endoderm, but which do not give rise to germ cells, sperm or egg.

Another category of stem cells is an adult stem cell which is any type of stem cell that is not derived from an embryo/fetus. For example, recent studies have indicated the presence of a more primitive cell population in the bone marrow capable of self-renewal as well as differentiation into a number of different tissue types other than blood cells. These multi-potential cells were discovered as a minor component in the CD34-plastic-adherent cell population of adult bone marrow, and are variously referred to as mesenchymal stem cells (MSC) (Pittenger, et al., Science 284:143-147 (1999)) or multi-potent adult progenitor cells (MAPC) cells (Furcht, L. T., et al., U.S. patent publication 20040107453 A1). MSC cells do not have a single specific identifying marker, but have been shown to be positive for a number of markers, including CD29, CD90, CD105, and CD73, and negative for other markers, including CD14, CD3, and CD34. Various groups have reported to differentiate MSC cells into myocytes, neurons, pancreatic beta-cells, liver cells, bone cells, and connective tissue. Another group (Wernet et al., U.S. patent publication 20020164794 A1) has described an unrestricted somatic stem cell (USSC) with multi-potential capacity that is derived from a CD45/CD34 population within cord blood. Typically, these stem cells have a limited capacity to generate new cell types and are committed to a particular lineage, although adult stem cells capable of generating all three cell types have been described (for example, United States Patent Application Publication No 20040107453 by Furcht, et al. published Jun. 3, 2004 and PCT/US02/04652, which are both incorporated by reference at least for material related to adult stem cells and culturing adult stem cells). An example of an adult stem cell is the multipotent hematopoietic stem cell, which forms all of the cells of the blood, such as erythrocytes, macrophages, T and B cells. Cells such as these are often referred to as “pluripotent hematopoietic stem cell” for its pluripotency within the hematopoietic lineage. A pluripotent adult stem cell is an adult stem cell having pluripotential capabilities (See for example, United States Patent Publication no. 20040107453, which is U.S. patent application Ser. No. 10/467,963).

Another category of stem cells is a blastocyst-derived stem cell which is a pluripotent stem cell which was derived from a cell which was obtained from a blastocyst prior to the, for example, 64, 100, or 150 cell stage. Blastocyst-derived stem cells can be derived from the inner cell mass of the blastocyst and are the cells commonly used in transgenic mouse work (Evans and Kaufman, (1981) Nature 292:154-156; Martin, (1981) Proc. Natl. Acad. Sci. 78:7634-7638). Blastocyst-derived stem cells isolated from cultured blastocysts can give rise to permanent cell lines that retain their undifferentiated characteristics indefinitely. Blastocyst-derived stem cells can be manipulated using any of the techniques of modern molecular biology, then re-implanted in a new blastocyst. This blastocyst can give rise to a full term animal carrying the genetic constitution of the blastocyst-derived stem cell. (Misra and Duncan, (2002) Endocrine 19:229-238). Such properties and manipulations are generally applicable to blastocyst-derived stem cells. It is understood blastocyst-derived stem cells can be obtained from pre or post implantation embryos and can be referred to as that there can be pre-implantation blastocyst-derived stem cells and post-implantation blastocyst-derived stem cells respectively.

Pluripotential stem cells can be isolated from fetal material, for example, from gonadal tissues, genital ridges, mesenteries or embryonic yolk sacs of embryos or fetal material. For example, such cells can be derived from primordial germ cells (PGCs). Pluripotential stem cells can also be derived from early embryos, such as blastocysts, testes (fetal and adult), and from other pluripotent stem cells such as ES and EG cells following the methods and using the compositions described herein.

The disclosed cells can lack the cell surface molecules required to substantially stimulate allogeneic lymphocytes in a mixed lymphocyte reaction. For example, the cells can lack the surface molecules required to substantially stimulate CD4+ T-cells in in vitro assessments, or in vivo in allogeneic, syngeneic, or autologous recipients. Preferably, the disclosed cells do not cause any substantial adverse immunological consequences for in vivo applications. For example, the therapeutic cell cultures can lack detectable amounts of at least two, or several, or all of the stimulating proteins HLA-DR, HLA-DP, HLA-DQ, CD80, CD86, and B7-H2, as determined by flow cytometry. Those lacking all of the foregoing are most preferred. Also preferred are therapeutic cell cultures which further lack detectable amounts of one or both of the immuno-modulating proteins HLA-G and CD178, as determined by flow cytometry. Also preferred are therapeutic cell cultures which express detectable amounts of the immuno-modulating protein PD-L2, as determined by flow cytometry. In one embodiment, the therapeutic cell culture does not substantially stimulate a lymphocyte mediated response in vitro, as compared to allogeneic controls in a mixed lymphocyte reaction.

### 3. Functional Nucleic Acids

Also disclosed herein are FG01 functional nucleic acid. In some aspects, the FG01 functional nucleic acid is an agonist of FG01 activity. In some aspects, the FG01 functional nucleic acid is an inhibitor of FG01 expression or activity. Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, RNAi, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA of FG01 or the genomic DNA of FG01 or they can interact with the polypeptide FG01. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (K_{d}) less than or equal to 10⁻⁶, 10⁻⁸, 10⁻¹⁰, or 10⁻¹². A representative sample of methods and techniques which aid in the design and use of antisense molecules can be found in U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with K_{d}'s from the target molecule of less than 10-12 M. It is preferred that the aptamers bind the target molecule with a K_{d} less than 10⁻⁶, 10⁻⁸, 10⁻¹⁰, or 10⁻¹². Aptamers can bind the target molecule with a very high degree of specificity. For example, aptamers have been isolated that have greater than a 10,000 fold difference in binding affinities between the target molecule and another molecule that differ at only a single position on the molecule (U.S. Pat. No. 5,543,293). It is preferred that the aptamer have a K_{d} with the target molecule at least 10, 100, 1000, 10,000, or 100,000 fold lower than the K_{d} with a background binding molecule. It is preferred when doing the comparison for a polypeptide for example, that the background molecule be a different polypeptide. Representative examples of how to make and use aptamers to bind a variety of different target molecules can be found in U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203; International Patent Application Nos. WO 9858058 by Ludwig and Sproat, WO 9858057 by Ludwig and Sproat, and WO 9718312 by Ludwig and Sproat) hairpin ribozymes (for example, U.S. Pat. Nos. 5,631,115, 5,646,031, 5,683,902, 5,712,384, 5,856,188, 5,866,701, 5,869,339, and 6,022,962), and tetrahymena ribozymes (for example, U.S. Pat. Nos. 5,595,873 and 5,652,107). There are also a number of ribozymes that are not found in natural systems, but which have been engineered to catalyze specific reactions de novo (for example, U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a K_{d} less than 10−6, 10−8, 10−10, or 10−12. Representative examples of how to make and use triplex forming molecules to bind a variety of different target molecules can be found in U.S. Pat. Nos. 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukarotic cells. (Yuan et al., Proc. Natl. Acad. Sci. USA 89:8006-8010 (1992); WO 93/22434 by Yale; WO 95/24489 by Yale; Yuan and Altman, EMBO J. 14:159-168 (1995), and Carrara et al., Proc. Natl. Acad. Sci. (USA) 92:2627-2631 (1995)). Representative examples of how to make and use EGS molecules to facilitate cleavage of a variety of different target molecules be found in U.S. Pat. Nos. 5,168,053, 5,624,824, 5,683,873, 5,728,521, 5,869,248, and 5,877,162.

Gene expression can also be effectively silenced in a highly specific manner through RNA interference (RNAi). This silencing was originally observed with the addition of double stranded RNA (dsRNA) (Fire, A., et al. (1998) Nature, 391:806-11; Napoli, C., et al. (1990) Plant Cell 2:279-89; Hannon, G. J. (2002) Nature, 418:244-51). Once dsRNA enters a cell, it is cleaved by an RNase III-like enzyme, Dicer, into double stranded small interfering RNAs (siRNA) 21-23 nucleotides in length that contains 2 nucleotide overhangs on the 3′ ends (Elbashir, S. M., et al. (2001) Genes Dev., 15:188-200; Bernstein, E., et al. (2001) Nature, 409:363-6; Hammond, S. M., et al. (2000) Nature, 404:293-6). In an ATP dependent step, the siRNAs become integrated into a multi-subunit protein complex, commonly known as the RNAi induced silencing complex (RISC), which guides the siRNAs to the target RNA sequence (Nykanen, A., et al. (2001) Cell, 107:309-21). At some point the siRNA duplex unwinds, and it appears that the antisense strand remains bound to RISC and directs degradation of the complementary mRNA sequence by a combination of endo and exonucleases (Martinez, J., et al. (2002) Cell, 110:563-74). However, the effect of iRNA or siRNA or their use is not limited to any type of mechanism.

Short Interfering RNA (siRNA) is a double-stranded RNA that can induce sequence-specific post-transcriptional gene silencing, thereby decreasing or even inhibiting gene expression. In one example, an siRNA triggers the specific degradation of homologous RNA molecules, such as mRNAs, within the region of sequence identity between both the siRNA and the target RNA. For example, WO 02/44321 discloses siRNAs capable of sequence-specific degradation of target mRNAs when base-paired with 3′ overhanging ends, herein incorporated by reference for the method of making these siRNAs. Sequence specific gene silencing can be achieved in mammalian cells using synthetic, short double-stranded RNAs that mimic the siRNAs produced by the enzyme dicer (Elbashir, S. M., et al. (2001) Nature, 411:494 498) (Ui-Tei, K., et al. (2000) FEBS Lett 479:79-82). siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. Suppliers include Ambion (Austin, Tex.), ChemGenes (Ashland, Mass.), Dharmacon (Lafayette, Colo.), Glen Research (Sterling, Va.), MWB Biotech (Esbersberg, Germany), Proligo (Boulder, Colo.), and Qiagen (Vento, The Netherlands). siRNA can also be synthesized in vitro using kits such as Ambion's SILENCER® siRNA Construction Kit. Disclosed herein are any siRNA designed as described above based on the sequences for FG01. For example, disclosed is an siRNA comprising the nucleic acid sequence SEQ ID NO:20.

The production of siRNA from a vector is more commonly done through the transcription of a short hairpin RNAs (shRNAs). Kits for the production of vectors comprising shRNA are available, such as, for example, Imgenex's GENESUPPRESSOR™ Construction Kits and Invitrogen's BLOCK-IT™ inducible RNAi plasmid and lentivirus vectors. Disclosed herein are any shRNA designed as described above based on the sequences for the herein disclosed inflammatory mediators.

### 4. Effectors

The herein provided compositions can further comprise an effector molecule. By “effector molecule” is meant a substance that acts upon the target cell(s) or tissue to bring about a desired effect. The effect can, for example, be the labeling, activating, repressing, or killing of the target cell(s) or tissue. Thus, the effector molecule can, for example, be a small molecule, pharmaceutical drug, toxin, fatty acid, detectable marker, conjugating tag, nanoparticle, or enzyme.

Examples of small molecules and pharmaceutical drugs that can be conjugated to a targeting peptide are known in the art. The effector can be a cytotoxic small molecule or drug that kills the target cell. The small molecule or drug can be designed to act on any critical cellular function or pathway. For example, the small molecule or drug can inhibit the cell cycle, activate protein degradation, induce apoptosis, modulate kinase activity, or modify cytoskeletal proteins. Any known or newly discovered cytotoxic small molecule or drugs is contemplated for use with the targeting peptides.

The effector can be a toxin that kills the targeted cell. Non-limiting examples of toxins include abrin, modeccin, ricin and diphtheria toxin. Other known or newly discovered toxins are contemplated for use with the provided compositions.

Fatty acids (i.e., lipids) that can be conjugated to the provided compositions include those that allow the efficient incorporation of the peptide into liposomes. Generally, the fatty acid is a polar lipid. Thus, the fatty acid can be a phospholipid The provided compositions can comprise either natural or synthetic phospholipid. The phospholipids can be selected from phospholipids containing saturated or unsaturated mono or disubstituted fatty acids and combinations thereof. These phospholipids can be dioleoylphosphatidylcholine, dioleoylphosphatidylserine, dioleoylphosphatidylethanolamine, dioleoylphosphatidylglycerol, dioleoylphosphatidic acid, palmitoyloleoylphosphatidylcholine, palmitoyloleoylphosphatidylserine, palmitoyloleoylphosphatidylethanolamine, palmitoyloleoylphophatidylglycerol, palmitoyloleoylphosphatidic acid, palmitelaidoyloleoylphosphatidylcholine, palmitelaidoyloleoylphosphatidylserine, palmitelaidoyloleoylphosphatidylethanolamine, palmitelaidoyloleoylphosphatidylglycerol, palmitelaidoyloleoylphosphatidic acid, myristoleoyloleoylphosphatidylcholine, myristoleoyloleoylphosphatidylserine, myristoleoyloleoylphosphatidylethanoamine, myristoleoyloleoylphosphatidylglycerol, myristoleoyloleoylphosphatidic acid, dilinoleoylphosphatidylcholine, dilinoleoylphosphatidylserine, dilinoleoylphosphatidylethanolamine, dilinoleoylphosphatidylglycerol, dilinoleoylphosphatidic acid, palmiticlinoleoylphosphatidylcholine, palmiticlinoleoylphosphatidylserine, palmiticlinoleoylphosphatidylethanolamine, palmiticlinoleoylphosphatidylglycerol, palmiticlinoleoylphosphatidic acid. These phospholipids may also be the monoacylated derivatives of phosphatidylcholine (lysophophatidylidylcholine), phosphatidylserine (lysophosphatidylserine), phosphatidylethanolamine (lysophosphatidylethanolamine), phophatidylglycerol (lysophosphatidylglycerol) and phosphatidic acid (lysophosphatidic acid). The monoacyl chain in these lysophosphatidyl derivatives may be palimtoyl, oleoyl, palmitoleoyl, linoleoyl myristoyl or myristoleoyl. The phospholipids can also be synthetic. Synthetic phospholipids are readily available commercially from various sources, such as AVANTI Polar Lipids (Albaster, Ala.); Sigma Chemical Company (St. Louis, Mo.). These synthetic compounds may be varied and may have variations in their fatty acid side chains not found in naturally occurring phospholipids. The fatty acid can have unsaturated fatty acid side chains with C14, C16, C18 or C20 chains length in either or both the PS or PC. Synthetic phospholipids can have dioleoyl (18:1)-PS; palmitoyl (16:0)-oleoyl (18:1)-PS, dimyristoyl (14:0)-PS; dipalmitoleoyl (16:1)-PC, dipalmitoyl (16:0)-PC, dioleoyl (18:1)-PC, palmitoyl (16:0)-oleoyl (18:1)-PC, and myristoyl (14:0)-oleoyl (18:1)-PC as constituents. Thus, as an example, the provided compositions can comprise palmitoyl 16:0.

Detectable markers include any substance that can be used to label or stain a target tissue or cell(s). Non-limiting examples of detectable markers include radioactive isotopes, enzymes, fluorochromes, and quantum dots (Qdot®). Other known or newly discovered detectable markers are contemplated for use with the provided compositions.

The effector molecule can be a nanoparticle, such as a heat generating nanoshell. As used herein, “nanoshell” is a nanoparticle having a discrete dielectric or semi-conducting core section surrounded by one or more conducting shell layers. U.S. Pat. No. 6,530,944 is hereby incorporated by reference herein in its entirety for its teaching of the methods of making and using metal nanoshells. Nanoshells can be formed with a core of a dielectric or inert material such as silicon, coated with a material such as a highly conductive metal which can be excited using radiation such as near infrared light (approximately 800 to 1300 nm). Upon excitation, the nanoshells emit heat. The resulting hyperthermia can kill the surrounding cell(s) or tissue. The combined diameter of the shell and core of the nanoshells ranges from the tens to the hundreds of nanometers. Near infrared light is advantageous for its ability to penetrate tissue. Other types of radiation can also be used, depending on the selection of the nanoparticle coating and targeted cells. Examples include x-rays, magnetic fields, electric fields, and ultrasound. The problems with the existing methods for hyperthermia, especially for use in cancer therapy, such as the use of heated probes, microwaves, ultrasound, lasers, perfusion, radiofrequency energy, and radiant heating is avoided since the levels of radiation used as described herein is insufficient to induce hyperthermia except at the surface of the nanoparticles, where the energy is more effectively concentrated by the metal surface on the dielectric. The particles can also be used to enhance imaging, especially using infrared diffuse photon imaging methods. Targeting molecules can be antibodies or fragments thereof, ligands for specific receptors, or other proteins specifically binding to the surface of the cells to be targeted.

The effector molecule can be a radioactive isotope. For example, the effector molecule can be a material, such as a “seed” or wire comprising any radioactive isotope suitable for implantation. A number of devices have been employed to implant radioactive seeds into tissues. See, e.g., U.S. Pat. Nos. 2,269,963 to Wappler; U.S. Pat. No. 4,402,308 to Scott; U.S. Pat. No. 5,860,909 to Mick; and U.S. Pat. No. 6,007,474 to Rydell. In a typical protocol for treating prostate cancer, an implantation device having a specialized needle is inserted through the skin between the rectum and scrotum into the prostate to deliver radioactive seeds to the prostate. The needle can be repositioned or a new needle used for other sites in the prostate where seeds are to be implanted. Typically, 20-40 needles are used to deliver between about 50-150 seeds per prostate. An ultrasound probe can be used to track the position of the needles.

Currently marketed radioactive seeds take the form of a capsule encapsulating a radioisotope. See, e.g., Symmetra® I-125 (Bebig GmbH, Germany); IoGold™ I-125 and IoGold™ Pd-103 (North American Scientific, Inc., Chatsworth, Calif.); Best® I-125 and Best Pd-103 (Best Industries, Springfield, Va.); Brachyseed® I-125 (Draximage, Inc., Canada); Intersource® Pd-103 (International Brachytherapy, Belgium); Oncoseed® I-125 (Nycomed Amersham, UK); STM 1250 I-125 (Sourcetech Medical, Carol Stream, Ill.); Pharmaseed® I-125 (Syncor, Woodland Hills, Calif.); Prostaseed™ I-125 (Urocor, Oklahoma City, Okla.); and I-Plant® I-125 (Implant Sciences Wakefield, Mass.). The capsule of these seeds can be made of a biocompatible substance such as titanium or stainless steel, and be tightly sealed to prevent leaching of the radioisotope. The capsule can be sized to fit down the bore of one of the needles used in the implantation device. Since most such needles are about 18 gauge, the capsule typically has a diameter of about 0.8 mm and a length of about 4.5-mm. The two radioisotopes most commonly used in brachytherapy seeds are iodine (I-¹²⁵) and palladium (Pd-¹⁰³). Both emit low energy irradiation and have half-life characteristics ideal for treating tumors. For example, I-¹²⁵ seeds decay at a rate of 50% every 60 days, so that using typical starting doses their radioactivity is almost exhausted after ten months. Pd-¹⁰³ seeds decay even more quickly, losing half their energy every 17 days so that they are nearly inert after only 3 months.

Radioactive brachytherapy seeds can also contain other components. For example, to assist in tracking their proper placement using standard X-ray imaging techniques, such seeds may contain a radiopaque marker. Markers are typically made of high atomic number (i.e., “high Z”) elements or alloys or mixtures containing such elements. Examples of these include platinum, iridium, rhenium, gold, tantalum, lead, bismuth alloys, indium alloys, solder or other alloys with low melting points, tungsten, and silver. Many radiopaque markers are currently being marketed including: platinum/iridium markers (Draximage, Inc. and International Brachytherapy), gold rods (Bebig GmbH), gold/copper alloy markers (North American Scientific), palladium rods (Syncor), tungsten markers (Best Industries), silver rods (Nycomed Amersham), silver spheres (International Isotopes Inc. and Urocor), and silver wire (Implant Sciences Corp.). Other radiopaque markers include polymers impregnated with various substances (see, e.g., U.S. Pat. No. 6,077,880).

A number of different U.S. patents disclose technology relating to brachytherapy. For example, U.S. Pat. No. 3,351,049 discloses the use of a low-energy X-ray-emitting interstitial implant as a brachytherapy source. In addition, U.S. Pat. No. 4,323,055; U.S. Pat. No. 4,702,228; U.S. Pat. No. 4,891,165; U.S. Pat. No. 5,405,309; U.S. Pat. No. 5,713,828; U.S. Pat. No. 5,997,463; U.S. Pat. Nos. 6,066,083; and 6,074,337 disclose technologies relating to brachytherapy devices.

The effector molecule can be covalently linked to the disclosed peptide. The effector molecule can be linked to the amino terminal end of the disclosed peptide. The effector molecule can be linked to the carboxy terminal end of the disclosed peptide. The effector molecule can be linked to an amino acid within the disclosed peptide. The herein provided compositions can further comprise a linker connecting the effector molecule and disclosed peptide. The disclosed peptide can also be conjugated to a coating molecule such as bovine serum albumin (BSA) (see Tkachenko et al., (2003) J Am Chem Soc, 125, 4700-4701) that can be used to coat the Nanoshells with the peptide.

The effector molecule can be covalently linked to the disclosed peptide. The effector molecule can be linked to the amino terminal end of the disclosed peptide. The effector molecule can be linked to the carboxy terminal end of the disclosed peptide. The effector molecule can be linked to an amino acid within the disclosed peptide. The herein provided compositions can further comprise a linker connecting the effector molecule and disclosed peptide. The disclosed peptide can also be conjugated to a coating molecule such as bovine serum albumin (BSA) (see Tkachenko et al., (2003) J Am Chem Soc, 125, 4700-4701) that can be used to coat the Nanoshells with the peptide.

Protein crosslinkers that can be used to crosslink the effector molecule to the disclosed peptide are known in the art and are defined based on utility and structure and include DSS (Disuccinimidylsuberate), DSP (Dithiobis(succinimidylpropionate)), DTSSP (3,3′-Dithiobis (sulfosuccinimidylpropionate)), SULFO BSOCOES (Bis[2-(sulfosuccinimdooxycarbonyloxy) ethyl]sulfone), BSOCOES (Bis[2-(succinimdooxycarbonyloxy)ethyl]sulfone), SULFO DST (Disulfosuccinimdyltartrate), DST (Disuccinimdyltartrate), SULFO EGS (Ethylene glycolbis(succinimidylsuccinate)), EGS (Ethylene glycolbis(sulfosuccinimidylsuccinate)), DPDPB (1,2-Di[3′-(2′-pyridyldithio) propionamido]butane), BSSS (Bis(sulfosuccinimdyl) suberate), SMPB (Succinimdyl-4-(p-maleimidophenyl) butyrate), SULFO SMPB (Sulfosuccinimdyl-4-(p-maleimidophenyl) butyrate), MBS (3-Maleimidobenzoyl-N-hydroxysuccinimide ester), SULFO MBS (3-Maleimidobenzoyl-N-hydroxysulfosuccinimide ester), SIAB (N-Succinimidyl(4-iodoacetyl) aminobenzoate), SULFO SIAB (N-Sulfosuccinimidyl(4-iodoacetyl)aminobenzoate), SMCC (Succinimidyl-4-(N-maleimidomethyl) cyclohexane-1-carboxylate), SULFO SMCC (Sulfosuccinimidyl-4-(N-maleimidomethyl) cyclohexane-1-carboxylate), NHS LC SPDP (Succinimidyl-6-[3-(2-pyridyldithio) propionamido) hexanoate), SULFO NHS LC SPDP (Sulfosuccinimidyl-6-[3-(2-pyridyldithio) propionamido) hexanoate), SPDP (N-Succinimdyl-3-(2-pyridyldithio)propionate), NHS BROMOACETATE (N-Hydroxysuccinimidylbromoacetate), NHS IODOACETATE (N-Hydroxysuccinimidyliodoacetate), MPBH (4-(N-Maleimidophenyl) butyric acid hydrazide hydrochloride), MCCH (4-(N-Maleimidomethyl) cyclohexane-1-carboxylic acid hydrazide hydrochloride), MBH (m-Maleimidobenzoic acid hydrazidehydrochloride), SULFO EMCS(N-(epsilon-Maleimidocaproyloxy) sulfosuccinimide), EMCS(N-(epsilon-Maleimidocaproyloxy) succinimide), PMPI (N-(p-Maleimidophenyl) isocyanate), KMUH (N-(kappa-Maleimidoundecanoic acid) hydrazide), LC SMCC (Succinimidyl-4-(N-maleimidomethyl)-cyclohexane-1-carboxy(6-amidocaproate)), SULFO GMBS (N-(gamma-Maleimidobutryloxy) sulfosuccinimide ester), SMPH (Succinimidyl-6-(beta-maleimidopropionamidohexanoate)), SULFO KMUS (N-(kappa-Maleimidoundecanoyloxy)sulfosuccinimide ester), GMBS (N-(gamma-Maleimidobutyrloxy) succinimide), DMP (Dimethylpimelimidate hydrochloride), DMS (Dimethylsuberimidate hydrochloride), MHBH (Wood's Reagent) (Methyl-p-hydroxybenzimidate hydrochloride, 98%), DMA (Dimethyladipimidate hydrochloride).

### 5. Sequence Similarities

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

### 6. Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

### 7. Combination Therapies

Provided herein is a composition that comprises FG01 and any known or newly discovered substance that can be administered to the site of a cancer, neurodegeneration, or metabolic disregulation. For example, the provided composition can further comprise one or more of classes of antibiotics (e.g. Aminoglycosides, Cephalosporins, Chloramphenicol, Clindamycin, Erythromycins, Fluoroquinolones, Macrolides, Azolides, Metronidazole, Penicillin's, Tetracycline's, Trimethoprim-sulfamethoxazole, Vancomycin), steroids (e.g. Andranes (e.g. Testosterone), Cholestanes (e.g. Cholesterol), Cholic acids (e.g. Cholic acid), Corticosteroids (e.g. Dexamethasone), Estraenes (e.g. Estradiol), Pregnanes (e.g. Progesterone), narcotic and non-narcotic analgesics (e.g. Morphine, Codeine, Heroin, Hydromorphone, Levorphanol, Meperidine, Methadone, Oxydone, Propoxyphene, Fentanyl, Methadone, Naloxone, Buprenorphine, Butorphanol, Nalbuphine, Pentazocine), anti-inflammatory agents (e.g. Alclofenac; Alclometasone Dipropionate; Algestone Acetonide; alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Decanoate; Deflazacort; Delatestryl; Depo-Testosterone; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Meclofenamate Sodium; Meclofenamic Acid; Meclorisone Dibutyrate; Mefenamic Acid; Mesalamine; Meseclazone; Mesterolone; Methandrostenolone; Methenolone; Methenolone Acetate; Methylprednisolone Suleptanate; Momiflumate; Nabumetone; Nandrolone; Naproxen; Naproxen Sodium; Naproxol; Nimazone; Olsalazine Sodium; Orgotein; Orpanoxin; Oxandrolane; Oxaprozin; Oxyphenbutazone; Oxymetholone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Stanozolol; Sudoxicam; Sulindac; Suprofen; Talmetacin; Talniflumate; Talosalate; Tebufelone; Tenidap; Tenidap Sodium; Tenoxicam; Tesicam; Tesimide; Testosterone; Testosterone Blends; Tetrydamine; Tiopinac; Tixocortol Pivalate; Tolmetin; Tolmetin Sodium; Triclonide; Triflumidate; Zidometacin; Zomepirac Sodium), or anti-histaminic agents (e.g. Ethanolamines (like diphenhydrmine carbinoxamine), Ethylenediamine (like tripelennamine pyrilamine), Alkylamine (like chlorpheniramine, dexchlorpheniramine, brompheniramine, triprolidine), other anti-histamines like astemizole, loratadine, fexofenadine, Bropheniramine, Clemastine, Acetaminophen, Pseudoephedrine, Triprolidine).

Numerous anti-cancer drugs are available for combination with the present method and compositions. The following are lists of anti-cancer (anti-neoplastic) drugs that can be used in conjunction with the presently disclosed DOC1 activity-enhancing or expression-enhancing methods.

Antineoplastic: Acivicin; Aclarubicin; Acodazole Hydrochloride; AcrQnine; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflomithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine; Fludarabine Phosphate; Fluorouracil; Fluorocitabine; Fosquidone; Fostriecin Sodium; Gemcitabine; Gemcitabine Hydrochloride; Gold Au 198; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safmgol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride.

Other anti-neoplastic compounds include: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; atrsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docosanol; dolasetron; doxifluridine; droloxifene; dronabinol; duocannycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; fmasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; irinotecan; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance genie inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfmosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thalidomide; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene dichloride; topotecan; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer.

The herein provide composition can further comprise one or more additional radiosensitizers. Examples of known radiosensitizers include gemcitabine, 5-fluorouracil, pentoxifylline, and vinorelbine. (Zhang et al., 1998; Lawrence et al., 2001; Robinson and Shewach, 2001; Strunz et al., 2002; Collis et al., 2003; Zhang et al., 2004).

The herein provide composition can further comprise Levodopa. The most widely used form of treatment is L-dopa in various forms. L-dopa is transformed into dopamine in the dopaminergic neurons by L-aromatic amino acid decarboxylase (often known by its former name dopa-decarboxylase). However, only 1-5% of L-DOPA enters the dopaminergic neurons. The remaining L-DOPA is often metabolised to dopamine elsewhere, causing a wide variety of side effects. Due to feedback inhibition, L-dopa results in a reduction in the endogenous formation of L-dopa, and so eventually becomes counterproductive.

The herein provide composition can further comprise Carbidopa or Benserazide. Carbidopa or Benserazide are dopa decarboxylase inhibitors. They help to prevent the metabolism of L-dopa before it reaches the dopaminergic neurons and are general given as combination preparations of carbidopa/levodopa (co-careldopa BAN) co-careldopa combined L-dopa and carbidopa in fixed ratios in such branded products of Sinemetand Parcopa and Benserazide/levodopa (co-beneldopa BAN) as Madopar. There are also controlled release versions of Sinemet and Madopar that spread out the effect of the L-dopa. Duodopa is a combination of levodopa and carbidopa, dispersed as a viscous gel. Using a patient-operated portable pump, the drug is continuously delivered via a tube directly into the upper small intestine, where it is rapidly absorbed.

The herein provide composition can further comprise Talcopone. Talcopone inhibits the COMT enzyme, thereby prolonging the effects of L-dopa, and so has been used to complement L-dopa. A similar drug, entacapone, has similar efficacy and has not been shown to cause significant alterations of liver function. Stalevo contains Levodopa, Carbidopa and Entacopone.

The herein provide composition can further comprise the dopamine-agonists bromocriptine (Parlodel), pergolide (Permax), pramipexole (Mirapex), ropinirole (Requip), cabergoline (Cabaser), apomorphine (Apokyn), or lisuride (Revanil). Dopamine agonists initially act by stimulating some of the dopamine receptors.

The herein provide composition can further comprise an MAO-B inhibitor. For example, selegiline (Eldepryl) and rasagiline (Azilect) reduce the symptoms by inhibiting monoamine oxidase-B (MAO-B), which inhibits the breakdown of dopamine secreted by the dopaminergic neurons. By-products of selegiline include amphetamine and methamphetamine, which can cause side effects such as insomnia.

The herein provide composition can further comprise a nucleic acid encoding glutamic acid decarboxylase (GAD), which catalyses the production of a neurotransmitter called GABA. GABA acts as a direct inhibitor on the overactive cells in the STN.

The herein provide composition can further comprise glial-derived neurotrophic factor (GDNF). Via a series of biochemical reactions, GDNF stimulates the formation of L-dopa.

The herein provide composition can further comprise an acetylcholinesterase inhibitor. Acetylcholinesterase inhibitors reduce the rate at which acetylcholine (ACh) is broken down and hence increase the concentration of ACh in the brain (combating the loss of ACh caused by the death of the cholinergin neurons). Examples currently marketed include donepezil (Aricept, Eisai and Pfizer), galantamine (Razadyne, Ortho-McNeil Neurologics, US) and rivastigmine (Exelon and Exelon Patch, Novartis). Donepezil and galantamine are taken orally. Rivastigmine has oral forms and a once-daily transdermal patch.

The herein provide composition can further comprise memantine (Namenda, Forest Pharmaceuticals, Axura, Merz GMBh, Ebixa, H. Lundbeck, and Akatinol). Memantine is a novel NMDA receptor antagonist, and has been shown to be moderately clinically efficacious.

The herein provide composition can further comprise one or more cells. The cell can be a stem cell. The stem cell can be a pluripotent stem cell. The cell can be a progenitor cell. The cell can be a neural progenitor cell. The cell can be a stem cell capable of differentiating into a neural cell. Thus, the herein provide composition can further comprise stem cells treated with factors to induce differentiation into neural cells. Other such cells known in the art for treating neurodegenerative disease or delivery of compositions to the brain are contemplated herein.

### 8. Antibodies

Also provided herein is an antibody that specifically binds FG01. Thus, provided herein is an antibody that specifically binds an amino acid consisting of the sequence SEQ ID NO:2.

Thus, also disclosed are immunodetection methods for detecting FG01 in a sample using the disclosed antibody. For example, the antibody can be used to identify proteins that bind FG01. The steps of various useful immunodetection methods have been described in the scientific literature, such as, e.g., Maggio et al., Enzyme-Immunoassay, (1987) and Nakamura, et al., Enzyme Immunoassays: Heterogeneous and Homogeneous Systems, Handbook of Experimental Immunology, Vol. 1: Immunochemistry, 27.1-27.20 (1986), each of which is incorporated herein by reference in its entirety and specifically for its teaching regarding immunodetection methods. Immunoassays, in their most simple and direct sense, are binding assays involving binding between antibodies and antigen. Many types and formats of immunoassays are known and all are suitable for detecting the disclosed biomarkers. Examples of immunoassays are enzyme linked immunosorbent assays (ELISAs), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP).

i. Antibodies Generally

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with FG01. The antibodies can be tested for their desired activity using the in vitro assays described herein, or by analogous methods, after which their in vivo therapeutic and/or prophylactic activities are tested according to known clinical testing methods.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity (See, U.S. Pat. No. 4,816,567 and Morrison et al., Proc. Natl. Acad. Sci. USA, 81:6851-6855 (1984)).

The disclosed monoclonal antibodies can be made using any procedure which produces mono clonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

If these approaches do not produce neutralizing antibodies, cells expressing cell surface localized versions of these proteins will be used to immunize mice, rats or other species. Traditionally, the generation of monoclonal antibodies has depended on the availability of purified protein or peptides for use as the immunogen. More recently DNA based immunizations have shown promise as a way to elicit strong immune responses and generate monoclonal antibodies. In this approach, DNA-based immunization can be used, wherein DNA encoding extracellular fragments of DR3 and TL1A expressed as a fusion protein with human IgG1 or an epitope tag is injected into the host animal according to methods known in the art (e.g., Kilpatrick K E, et al. Gene gun delivered DNA-based immunizations mediate rapid production of murine monoclonal antibodies to the Flt-3 receptor. Hybridoma. 1998 December; 17(6):569-76; Kilpatrick K E et al. High-affinity monoclonal antibodies to PED/PEA-15 generated using 5 microg of DNA. Hybridoma. 2000 August; 19(4):297-302, which are incorporated herein by referenced in full for the methods of antibody production) and as described in the examples.

An alternate approach to immunizations with either purified protein or DNA is to use antigen expressed in baculovirus. The advantages to this system include ease of generation, high levels of expression, and post-translational modifications that are highly similar to those seen in mammalian systems. Use of this system involves expressing the extracellular domain of TL1A or DR3 as fusion proteins with a signal sequence fragment. The antigen is produced by inserting a gene fragment in-frame between the signal sequence and the mature protein domain of the TL1A or DR3 nucleotide sequence. This results in the display of the foreign proteins on the surface of the virion. This method allows immunization with whole virus, eliminating the need for purification of target antigens.

Generally, either peripheral blood lymphocytes (“PBLs”) are used in methods of producing monoclonal antibodies if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, “Monoclonal Antibodies: Principles and Practice” Academic Press, (1986) pp. 59-103). Immortalized cell lines are usually transformed mammalian cells, including myeloma cells of rodent, bovine, equine, and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells. Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Rockville, Md. Human myeloma and mouse-human heteromyeloma cell lines also have been described for the production of human monoclonal antibodies (Kozbor, J. Immunol., 133:3001 (1984); Brodeur et al., “Monoclonal Antibody Production Techniques and Applications” Marcel Dekker, Inc., New York, (1987) pp. 51-63). The culture medium in which the hybridoma cells are cultured can then be assayed for the presence of monoclonal antibodies directed against DR3 and/or TL1A. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art, and are described further in the Examples below or in Harlow and Lane “Antibodies, A Laboratory Manual” Cold Spring Harbor Publications, New York, (1988).

After the desired hybridoma cells are identified, the clones may be subcloned by limiting dilution or FACS sorting procedures and grown by standard methods. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, protein G, hydroxylapatite chromatography, gel electrophoresis, dialysis, or affinity chromatography.

The monoclonal antibodies may also be made by recombinant DNA methods, such as those described in U.S. Pat. No. 4,816,567 (Cabilly et al.). DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

ii. Whole Immunoglobulin

As used herein, the term “antibody” encompasses, but is not limited to, whole immunoglobulin (i.e., an intact antibody) of any class. Native antibodies are usually heterotetrameric glycoproteins, composed of two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V(H)) followed by a number of constant domains. Each light chain has a variable domain at one end (V(L)) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains. The light chains of antibodies from any vertebrate species can be assigned to one of two clearly distinct types, called kappa (k) and lambda (l), based on the amino acid sequences of their constant domains. Depending on the amino acid sequence of the constant domain of their heavy chains, immunoglobulins can be assigned to different classes. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “variable” is used herein to describe certain portions of the variable domains that differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a b-sheet configuration, connected by three CDRs, which form loops connecting, and in some cases forming part of, the b-sheet structure. The CDRs in each chain are held together in close proximity by the FR regions and, with the CDRs from the other chain, contribute to the formation of the antigen binding site of antibodies (see Kabat E. A. et al., “Sequences of Proteins of Immunological Interest,” National Institutes of Health, Bethesda, Md. (1987)). The constant domains are not involved directly in binding an antibody to an antigen, but exhibit various effector functions, such as participation of the antibody in antibody-dependent cellular toxicity.

iii. Antibody Fragments

The term “antibody” as used herein is meant to include intact molecules as well as fragments thereof, such as, for example, Fab and F(ab′)₂, which are capable of binding the epitopic determinant.

As used herein, the term “antibody or fragments thereof” encompasses chimeric antibodies and hybrid antibodies, with dual or multiple antigen or epitope specificities, and fragments, such as F(ab′)2, Fab′, Fab and the like, including hybrid fragments. Thus, fragments of the antibodies that retain the ability to bind their specific antigens are provided. For example, fragments of antibodies which maintain DR3 or TL1A binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies) as described, for example, in U.S. Pat. No. 4,704,692, the contents of which are hereby incorporated by reference.

An isolated immunogenically specific paratope or fragment of the antibody is also provided. A specific immunogenic epitope of the antibody can be isolated from the whole antibody by chemical or mechanical disruption of the molecule. The purified fragments thus obtained are tested to determine their immunogenicity and specificity by the methods taught herein. Immunoreactive paratopes of the antibody, optionally, are synthesized directly. An immunoreactive fragment is defined as an amino acid sequence of at least about two to five consecutive amino acids derived from the antibody amino acid sequence.

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

Also disclosed are fragments of antibodies which have bioactivity. The polypeptide fragments can be recombinant proteins obtained by cloning nucleic acids encoding the polypeptide in an expression system capable of producing the polypeptide fragments thereof, such as an adenovirus or baculovirus expression system. For example, one can determine the active domain of an antibody from a specific hybridoma that can cause a biological effect associated with the interaction of the antibody with TL1A or DR3. For example, amino acids found to not contribute to either the activity or the binding specificity or affinity of the antibody can be deleted without a loss in the respective activity. For example, in various embodiments, amino or carboxy-terminal amino acids are sequentially removed from either the native or the modified non-immunoglobulin molecule or the immunoglobulin molecule and the respective activity assayed in one of many available assays. In another example, a fragment of an antibody comprises a modified antibody wherein at least one amino acid has been substituted for the naturally occurring amino acid at a specific position, and a portion of either amino terminal or carboxy terminal amino acids, or even an internal region of the antibody, has been replaced with a polypeptide fragment or other moiety, such as biotin, which can facilitate in the purification of the modified antibody. For example, a modified antibody can be fused to a maltose binding protein, through either peptide chemistry or cloning the respective nucleic acids encoding the two polypeptide fragments into an expression vector such that the expression of the coding region results in a hybrid polypeptide. The hybrid polypeptide can be affinity purified by passing it over an amylose affinity column, and the modified antibody receptor can then be separated from the maltose binding region by cleaving the hybrid polypeptide with the specific protease factor Xa. (See, for example, New England Biolabs Product Catalog, 1996, pg. 164). Similar purification procedures are available for isolating hybrid proteins from eukaryotic cells as well.

The fragments, whether attached to other sequences or not, include insertions, deletions, substitutions, or other selected modifications of particular regions or specific amino acids residues, provided the activity of the fragment is not significantly altered or impaired compared to the nonmodified antibody or antibody fragment. These modifications can provide for some additional property, such as to remove or add amino acids capable of disulfide bonding, to increase its bio-longevity, to alter its secretory characteristics, etc. In any case, the fragment must possess a bioactive property, such as binding activity, regulation of binding at the binding domain, etc. Functional or active regions of the antibody may be identified by mutagenesis of a specific region of the protein, followed by expression and testing of the expressed polypeptide. Such methods are readily apparent to a skilled practitioner in the art and can include site-specific mutagenesis of the nucleic acid encoding the antigen. (Zoller M J et al. Nucl. Acids Res. 10:6487-500 (1982).

Techniques can also be adapted for the production of single-chain antibodies specific to an antigenic protein of the present disclosure (see e.g., U.S. Pat. No. 4,946,778). In addition, methods can be adapted for the construction of F (ab) expression libraries (see e.g., Huse, et al., 1989 Science 246: 1275-1281) to allow rapid and effective identification of monoclonal F (ab) fragments with the desired specificity for a protein or derivatives, fragments, analogs or homologs thereof. Antibody fragments that contain the idiotypes to a protein antigen may be produced by techniques known in the art including, but not limited to: (i) an F ((ab′))(2) fragment produced by pepsin digestion of an antibody molecule; (ii) an Fab fragment generated by reducing the disulfide bridges of an F ((ab′))(2) fragment; (iii) an F (ab) fragment generated by the treatment of the antibody molecule with papain and a reducing agent and (iv) F (v), fragments.

Methods for the production of single-chain antibodies are well known to those of skill in the art. The skilled artisan is referred to U.S. Pat. No. 5,359,046, (incorporated herein by reference) for such methods. A single chain antibody is created by fusing together the variable domains of the heavy and light chains using a short peptide linker, thereby reconstituting an antigen binding site on a single molecule. Single-chain antibody variable fragments (scFvs) in which the C-terminus of one variable domain is tethered to the N-terminus of the other variable domain via a 15 to 25 amino acid peptide or linker have been developed without significantly disrupting antigen binding or specificity of the binding (Bedzyk et al., 1990; Chaudhary et al., 1990). The linker is chosen to permit the heavy chain and light chain to bind together in their proper conformational orientation. See, for example, Huston, J. S., et al., Methods in Enzym. 203:46-121 (1991), which is incorporated herein by reference. These Fvs lack the constant regions (Fc) present in the heavy and light chains of the native antibody.

iv. Monovalent Antibodies

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994, U.S. Pat. No. 4,342,566, and Harlow and Lane, Antibodies, A Laboratory Manual, Cold Spring Harbor Publications, New York, (1988). Papain digestion of antibodies typically produces two identical antigen binding fragments, called Fab fragments, each with a single antigen binding site, and a residual Fc fragment. Pepsin treatment yields a fragment, called the F(ab′)2 fragment, that has two antigen combining sites and is still capable of cross-linking antigen.

The Fab fragments produced in the antibody digestion also contain the constant domains of the light chain and the first constant domain of the heavy chain. Fab′ fragments differ from Fab fragments by the addition of a few residues at the carboxy terminus of the heavy chain domain including one or more cysteines from the antibody hinge region. The F(ab′)2 fragment is a bivalent fragment comprising two Fab′ fragments linked by a disulfide bridge at the hinge region. Fab′-SH is the designation herein for Fab′ in which the cysteine residue(s) of the constant domains bear a free thiol group. Antibody fragments originally were produced as pairs of Fab′ fragments which have hinge cysteines between them. Other chemical couplings of antibody fragments are also known.

v. Chimeric/Hybrid

In hybrid antibodies, one heavy and light chain pair is homologous to that found in an antibody raised against one antigen recognition feature, e.g., epitope, while the other heavy and light chain pair is homologous to a pair found in an antibody raised against another epitope. This results in the property of multi-functional valency, i.e., ability to bind at least two different epitopes simultaneously. As used herein, the term “hybrid antibody” refers to an antibody wherein each chain is separately homologous with reference to a mammalian antibody chain, but the combination represents a novel assembly so that two different antigens are recognized by the antibody. Such hybrids can be formed by fusion of hybridomas producing the respective component antibodies, or by recombinant techniques. Such hybrids may, of course, also be formed using chimeric chains.

vi. Anti-Idiotypic

The encoded antibodies can be anti-idiotypic antibodies (antibodies that bind other antibodies) as described, for example, in U.S. Pat. No. 4,699,880. Such anti-idiotypic antibodies could bind endogenous or foreign antibodies in a treated individual, thereby to ameliorate or prevent pathological conditions associated with an immune response, e.g., in the context of an autoimmune disease.

vii. Conjugates or Fusions of Antibody Fragments

The targeting function of the antibody can be used therapeutically by coupling the antibody or a fragment thereof with a therapeutic agent. Such coupling of the antibody or fragment (e.g., at least a portion of an immunoglobulin constant region (Fc)) with the therapeutic agent can be achieved by making an immunoconjugate or by making a fusion protein, comprising the antibody or antibody fragment and the therapeutic agent. For example, provided is a DR3 Fc fusion protein, e.g., DR3 extracellular domain (150 aa) fused to mouse IgG Fc (DR3 (human)-muIg Fusion Protein).

Also included within the meaning of “antibody or fragments thereof” are conjugates of antibody fragments and antigen binding proteins (single chain antibodies) as described, for example, in U.S. Pat. No. 4,704,692, the contents of which are hereby incorporated by reference.

An antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates disclosed can be used for modifying a given biological response. The drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, α-interferon, β-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982). Alternatively, an antibody can be conjugated to a second antibody to form an antibody heteroconjugate as described by Segal in U.S. Pat. No. 4,676,980.

viii. Method of Making Antibodies Using Protein Chemistry

One method of producing proteins comprising the antibodies is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the antibody, for example, can be synthesized by standard chemical reactions. For example, a peptide or polypeptide can be synthesized and not cleaved from its synthesis resin whereas the other fragment of an antibody can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant G A (1992) Synthetic Peptides: A User Guide. W.H. Freeman and Co., N.Y. (1992); Bodansky M and Trost B., Ed. (1993) Principles of Peptide Synthesis. Springer-Verlag Inc., NY. Alternatively, the peptide or polypeptide is independently synthesized in vivo as described above. Once isolated, these independent peptides or polypeptides may be linked to form an antibody or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide-alpha-thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site. Application of this native chemical ligation method to the total synthesis of a protein molecule is illustrated by the preparation of human interleukin 8 (IL-8) (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

ix. Human and Humanized

Transgenic animals (e.g., mice) that are capable, upon immunization, of producing a full repertoire of human antibodies in the absence of endogenous immunoglobulin production can be employed. For example, it has been described that the homozygous deletion of the antibody heavy chain joining region (J(H)) gene in chimeric and germ-line mutant mice results in complete inhibition of endogenous antibody production. Transfer of the human germ-line immunoglobulin gene array in such germ-line mutant mice will result in the production of human antibodies upon antigen challenge (see, e.g., Jakobovits et al., Proc. Natl. Acad. Sci. USA, 90:2551-255 (1993); Jakobovits et al., Nature, 362:255-258 (1993); Bruggemann et al., Year in Immuno., 7:33 (1993)). Human antibodies can also be produced in phage display libraries (Hoogenboom et al., J. Mol. Biol., 227:381 (1991); Marks et al., J. Mol. Biol., 222:581 (1991)). The techniques of Cote et al. and Boerner et al. are also available for the preparation of human monoclonal antibodies (Cole et al., Monoclonal Antibodies and Cancer Therapy, Alan R. Liss, p. 77 (1985); Boerner et al., J. Immunol., 147(1):86-95 (1991)).

Optionally, the antibodies are generated in other species and “humanized” for administration in humans. Humanized forms of non-human (e.g., murine) antibodies are chimeric immunoglobulins, immunoglobulin chains or fragments thereof (such as Fv, Fab, Fab′, F(ab′)2, or other antigen-binding subsequences of antibodies) which contain minimal sequence derived from non-human immunoglobulin. Humanized antibodies include human immunoglobulins (recipient antibody) in which residues from a complementarity determining region (CDR) of the recipient antibody are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues that are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature, 321:522-525 (1986); Riechmann et al., Nature, 332:323-327 (1988); and Presta, Curr. Op. Struct. Biol., 2:593-596 (1992))

Methods for humanizing non-human antibodies are well known in the art. Generally, a humanized antibody has one or more amino acid residues introduced into it from a source that is non-human. These non-human amino acid residues are often referred to as “import” residues, which are typically taken from an “import” variable domain. Antibody humanization techniques generally involve the use of recombinant DNA technology to manipulate the DNA sequence encoding one or more polypeptide chains of an antibody molecule. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, a humanized form of a non-human antibody (or a fragment thereof) is a chimeric antibody or fragment (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important in order to reduce antigenicity. According to the “best-fit” method, the sequence of the variable domain of a rodent antibody is screened against the entire library of known human variable domain sequences. The human sequence which is closest to that of the rodent is then accepted as the human framework (FR) for the humanized antibody (Sims et al., J. Immunol., 151:2296 (1993) and Chothia et al., J. Mol. Biol., 196:901 (1987)). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three dimensional models of the parental and humanized sequences. Three dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved. In general, the CDR residues are directly and most substantially involved in influencing antigen binding (see, WO 94/04679, published 3 Mar. 1994).

As used herein, the term “epitope” is meant to include any determinant capable of specific interaction with the anti-DR3 or anti-TL1A antibodies disclosed. Epitopic determinants usually consist of chemically active surface groupings of molecules such as amino acids or sugar side chains and usually have specific three dimensional structural characteristics, as well as specific charge characteristics.

An “epitope tag” denotes a short peptide sequence unrelated to the function of the antibody or molecule that can be used for purification or crosslinking of the molecule with anti-epitope tag antibodies or other reagents.

By “specifically binds” is meant that an antibody recognizes and physically interacts with its cognate antigen (e.g., a DR3 receptor polypeptide or a TL1A poly peptide) and does not significantly recognize and interact with other antigens; such an antibody may be a polyclonal antibody or a monoclonal antibody, which are generated by techniques that are well known in the art.

The antibody can be bound to a substrate or labeled with a detectable moiety or both bound and labeled. The detectable moieties contemplated with the present compositions include fluorescent, enzymatic and radioactive markers.

### 9. Cell Delivery Systems

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

i. Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed nucleic acids into the cell without degradation and include a promoter yielding expression of the gene in the cells into which it is delivered. In some embodiments the vectors are derived from either a virus or a retrovirus. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

a. Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

b. Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. In another preferred embodiment both the E1 and E3 genes are removed from the adenovirus genome.

c. Adeno-Associated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

The disclosed vectors thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

d. Large Payload Viral Vectors

Molecular genetic experiments with large human herpesviruses have provided a means whereby large heterologous DNA fragments can be cloned, propagated and established in cells permissive for infection with herpesviruses (Sun et al., Nature genetics 8: 33-41, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA >150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA >220 kb and to infect cells that can stably maintain DNA as episomes.

Other useful systems include, for example, replicating and host-restricted non-replicating vaccinia virus vectors.

Nucleic acids that are delivered to cells which are to be integrated into the host cell genome, typically contain integration sequences. These sequences are often viral related sequences, particularly when viral based systems are used. These viral intergration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

ii. Non-Nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci. USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue, the principles of which can be applied to targeting of other cells (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that are delivered to cells which are to be integrated into the host cell genome, typically contain integration sequences. These sequences are often viral related sequences, particularly when viral based systems are used. These viral intergration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

### 10. Carriers

The disclosed compositions comprising FG01, nucleic acids encoding FG01, or fragments thereof, can be combined, conjugated or coupled with or to carriers and other compositions to aid administration, delivery or other aspects of the inhibitors and their use. For convenience, such composition will be referred to herein as carriers. Carriers can, for example, be a small molecule, pharmaceutical drug, fatty acid, detectable marker, conjugating tag, nanoparticle, or enzyme.

The disclosed compositions can be used therapeutically in combination with a pharmaceutically acceptable carrier. By “pharmaceutically acceptable” is meant a material that is not biologically or otherwise undesirable, i.e., the material can be administered to a subject, along with the composition, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds can be administered according to standard procedures used by those skilled in the art.

Pharmaceutical compositions can include carriers, thickeners, diluents, buffers, preservatives, surface active agents and the like in addition to the molecule of choice. Pharmaceutical compositions can also include one or more active ingredients such as antimicrobial agents, antiinflammatory agents, anesthetics, and the like.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Formulations for topical administration can include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions can potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

The materials may be in solution, suspension (for example, incorporated into microparticles, liposomes, or cells). These can be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

The carrier molecule can be covalently linked to the disclosed inhibitors. The carrier molecule can be linked to the amino terminal end of the disclosed peptides. The carrier molecule can be linked to the carboxy terminal end of the disclosed peptides. The carrier molecule can be linked to an amino acid within the disclosed peptides. The herein provided compositions can further comprise a linker connecting the carrier molecule and disclosed inhibitors. The disclosed inhibitors can also be conjugated to a coating molecule such as bovine serum albumin (BSA) (see Tkachenko et al., (2003) J Am Chem Soc, 125, 4700-4701) that can be used to coat microparticles, nanoparticles of nanoshells with the inhibitors.

Protein crosslinkers that can be used to crosslink the carrier molecule to the inhibitors, such as the disclosed peptides, are known in the art and are defined based on utility and structure and include DSS (Disuccinimidylsuberate), DSP (Dithiobis(succinimidylpropionate)), DTSSP (3,3′-Dithiobis (sulfosuccinimidylpropionate)), SULFO BSOCOES (Bis[2-(sulfosuccinimdooxycarbonyloxy) ethyl]sulfone), BSOCOES (Bis[2-(succinimdooxycarbonyloxy)ethyl]sulfone), SULFO DST (Disulfosuccinimdyltartrate), DST (Disuccinimdyltartrate), SULFO EGS (Ethylene glycolbis(succinimidylsuccinate)), EGS (Ethylene glycolbis(sulfosuccinimidylsuccinate)), DPDPB (1,2-Di[3′-(2′-pyridyldithio) propionamido]butane), BSSS (Bis(sulfosuccinimdyl) suberate), SMPB (Succinimdyl-4-(p-maleimidophenyl) butyrate), SULFO SMPB (Sulfosuccinimdyl-4-(p-maleimidophenyl) butyrate), MBS (3-Maleimidobenzoyl-N-hydroxysuccinimide ester), SULFO MBS (3-Maleimidobenzoyl-N-hydroxysulfosuccinimide ester), SIAB (N-Succinimidyl(4-iodoacetyl) aminobenzoate), SULFO SIAB (N-Sulfosuccinimidyl(4-iodoacetyl)aminobenzoate), SMCC (Succinimidyl-4-(N-maleimidomethyl)cyclohexane-1-carboxylate), SULFO SMCC (Sulfosuccinimidyl-4-(N-maleimidomethyl)cyclohexane-1-carboxylate), NHS LC SPDP (Succinimidyl-6-[3-(2-pyridyldithio) propionamido) hexanoate), SULFO NHS LC SPDP (Sulfosuccinimidyl-6-[3-(2-pyridyldithio) propionamido) hexanoate), SPDP (N-Succinimdyl-3-(2-pyridyldithio) propionate), NHS BROMOACETATE (N-Hydroxysuccinimidylbromoacetate), NHS IODOACETATE (N-Hydroxysuccinimidyliodoacetate), MPBH (4-(N-Maleimidophenyl) butyric acid hydrazide hydrochloride), MCCH (4-(N-Maleimidomethyl)cyclohexane-1-carboxylic acid hydrazide hydrochloride), MBH (m-Maleimidobenzoic acid hydrazidehydrochloride), SULFO EMCS(N-(epsilon-Maleimidocaproyloxy) sulfosuccinimide), EMCS(N-(epsilon-Maleimidocaproyloxy) succinimide), PMPI (N-(p-Maleimidophenyl) isocyanate), KMUH (N-(kappa-Maleimidoundecanoic acid) hydrazide), LC SMCC (Succinimidyl-4-(N-maleimidomethyl)-cyclohexane-1-carboxy(6-amidocaproate)), SULFO GMBS (N-(gamma-Maleimidobutryloxy) sulfosuccinimide ester), SMPH (Succinimidyl-6-(beta-maleimidopropionamidohexanoate)), SULFO KMUS (N-(kappa-Maleimidoundecanoyloxy)sulfosuccinimide ester), GMBS (N-(gamma-Maleimidobutyrloxy) succinimide), DMP (Dimethylpimelimidate hydrochloride), DMS (Dimethylsuberimidate hydrochloride), MHBH (Wood's Reagent) (Methyl-p-hydroxybenzimidate hydrochloride, 98%), DMA (Dimethyladipimidate hydrochloride).

i. Nanoparticles, Microparticles, and Microbubbles

The term “nanoparticle” refers to a nanoscale particle with a size that is measured in nanometers, for example, a nanoscopic particle that has at least one dimension of less than about 100 nm. Examples of nanoparticles include paramagnetic nanoparticles, superparamagnetic nanoparticles, metal nanoparticles, fullerene-like materials, inorganic nanotubes, dendrimers (such as with covalently attached metal chelates), nanofibers, nanohoms, nano-onions, nanorods, nanoropes and quantum dots. A nanoparticle can produce a detectable signal, for example, through absorption and/or emission of photons (including radio frequency and visible photons) and plasmon resonance.

Microspheres (or microbubbles) can also be used with the methods disclosed herein. Microspheres containing chromophores have been utilized in an extensive variety of applications, including photonic crystals, biological labeling, and flow visualization in microfluidic channels. See, for example, Y. Lin, et al., Appl. Phys Lett. 2002, 81, 3134; D. Wang, et al., Chem. Mater. 2003, 15, 2724; X. Gao, et al., J. Biomed. Opt. 2002, 7, 532; M. Han, et al., Nature Biotechnology. 2001, 19, 631; V. M. Pai, et al., Mag. & Magnetic Mater. 1999, 194, 262, each of which is incorporated by reference in its entirety. Both the photostability of the chromophores and the monodispersity of the microspheres can be important.

Nanoparticles, such as, for example, silica nanoparticles, metal nanoparticles, metal oxide nanoparticles, or semiconductor nanocrystals can be incorporated into microspheres. The optical, magnetic, and electronic properties of the nanoparticles can allow them to be observed while associated with the microspheres and can allow the microspheres to be identified and spatially monitored. For example, the high photostability, good fluorescence efficiency and wide emission tunability of colloidally synthesized semiconductor nanocrystals can make them an excellent choice of chromophore. Unlike organic dyes, nanocrystals that emit different colors (i.e. different wavelengths) can be excited simultaneously with a single light source. Colloidally synthesized semiconductor nanocrystals (such as, for example, core-shell CdSe/ZnS and CdS/ZnS nanocrystals) can be incorporated into microspheres. The microspheres can be monodisperse silica microspheres.

The nanoparticle can be a metal nanoparticle, a metal oxide nanoparticle, or a semiconductor nanocrystal. The metal of the metal nanoparticle or the metal oxide nanoparticle can include titanium, zirconium, hafnium, vanadium, niobium, tantalum, chromium, molybdenum, tungsten, manganese, technetium, rhenium, iron, ruthenium, osmium, cobalt, rhodium, iridium, nickel, palladium, platinum, copper, silver, gold, zinc, cadmium, scandium, yttrium, lanthanum, a lanthanide series or actinide series element (e.g., cerium, praseodymium, neodymium, promethium, samarium, europium, gadolinium, terbium, dysprosium, holmium, erbium, thulium, ytterbium, lutetium, thorium, protactinium, and uranium), boron, aluminum, gallium, indium, thallium, silicon, germanium, tin, lead, antimony, bismuth, polonium, magnesium, calcium, strontium, and barium. In certain embodiments, the metal can be iron, ruthenium, cobalt, rhodium, nickel, palladium, platinum, silver, gold, cerium or samarium. The metal oxide can be an oxide of any of these materials or combination of materials. For example, the metal can be gold, or the metal oxide can be an iron oxide, a cobalt oxide, a zinc oxide, a cerium oxide, or a titanium oxide. Preparation of metal and metal oxide nanoparticles is described, for example, in U.S. Pat. Nos. 5,897,945 and 6,759,199, each of which is incorporated by reference in its entirety.

For example, the disclosed compositions comprising FG01, nucleic acids encoding FG01, or fragments thereof, can be immobilized on silica nanoparticles (SNPs). SNPs have been widely used for biosensing and catalytic applications owing to their favorable surface area-to-volume ratio, straightforward manufacture and the possibility of attaching fluorescent labels, magnetic nanoparticles (Yang, H. H. et al. 2005) and semiconducting nanocrystals (Lin, Y. W., et al. 2006).

The nanoparticle can also be, for example, a heat generating nanoshell. As used herein, “nanoshell” is a nanoparticle having a discrete dielectric or semi-conducting core section surrounded by one or more conducting shell layers. U.S. Pat. No. 6,530,944 is hereby incorporated by reference herein in its entirety for its teaching of the methods of making and using metal nanoshells.

Targeting molecules can be attached to the disclosed compositions and/or carriers. For example, the targeting molecules can be antibodies or fragments thereof, ligands for specific receptors, or other proteins specifically binding to the surface of the cells to be targeted.

ii. Liposomes

“Liposome” as the term is used herein refers to a structure comprising an outer lipid bi- or multi-layer membrane surrounding an internal aqueous space. Liposomes can be used to package any biologically active agent for delivery to cells.

Materials and procedures for forming liposomes are well-known to those skilled in the art. Upon dispersion in an appropriate medium, a wide variety of phospholipids swell, hydrate and form multilamellar concentric bilayer vesicles with layers of aqueous media separating the lipid bilayers. These systems are referred to as multilamellar liposomes or multilamellar lipid vesicles (“MLVs”) and have diameters within the range of 10 nm to 100 μm. These MLVs were first described by Bangham, et al., J Mol. Biol. 13:238-252 (1965). In general, lipids or lipophilic substances are dissolved in an organic solvent. When the solvent is removed, such as under vacuum by rotary evaporation, the lipid residue forms a film on the wall of the container. An aqueous solution that typically contains electrolytes or hydrophilic biologically active materials is then added to the film. Large MLVs are produced upon agitation. When smaller MLVs are desired, the larger vesicles are subjected to sonication, sequential filtration through filters with decreasing pore size or reduced by other forms of mechanical shearing. There are also techniques by which MLVs can be reduced both in size and in number of lamellae, for example, by pressurized extrusion (Barenholz, et al., FEBS Lett. 99:210-214 (1979)).

Liposomes can also take the form of unilamnellar vesicles, which are prepared by more extensive sonication of MLVs, and consist of a single spherical lipid bilayer surrounding an aqueous solution. Unilamellar vesicles (“ULVs”) can be small, having diameters within the range of 20 to 200 nm, while larger ULVs can have diameters within the range of 200 nm to 2 μm. There are several well-known techniques for making unilamellar vesicles. In Papahadjopoulos, et al., Biochim et Biophys Acta 135:624-238 (1968), sonication of an aqueous dispersion of phospholipids produces small ULVs having a lipid bilayer surrounding an aqueous solution. Schneider, U.S. Pat. No. 4,089,801 describes the formation of liposome precursors by ultrasonication, followed by the addition of an aqueous medium containing amphiphilic compounds and centrifugation to form a biomolecular lipid layer system.

Small ULVs can also be prepared by the ethanol injection technique described by Batzri, et al., Biochim et Biophys Acta 298:1015-1019 (1973) and the ether injection technique of Deamer, et al., Biochim et Biophys Acta 443:629-634 (1976). These methods involve the rapid injection of an organic solution of lipids into a buffer solution, which results in the rapid formation of unilamellar liposomes. Another technique for making ULVs is taught by Weder, et al. in “Liposome Technology”, ed. G. Gregoriadis, CRC Press Inc., Boca Raton, Fla., Vol. I, Chapter 7, pg. 79-107 (1984). This detergent removal method involves solubilizing the lipids and additives with detergents by agitation or sonication to produce the desired vesicles.

Papahadjopoulos, et al., U.S. Pat. No. 4,235,871, describes the preparation of large ULVs by a reverse phase evaporation technique that involves the formation of a water-in-oil emulsion of lipids in an organic solvent and the drug to be encapsulated in an aqueous buffer solution. The organic solvent is removed under pressure to yield a mixture which, upon agitation or dispersion in an aqueous media, is converted to large ULVs. Suzuki et al., U.S. Pat. No. 4,016,100, describes another method of encapsulating agents in unilamellar vesicles by freezing/thawing an aqueous phospholipid dispersion of the agent and lipids.

In addition to the MLVs and ULVs, liposomes can also be multivesicular. Described in Kim, et al., Biochim et Biophys Acta 728:339-348 (1983), these multivesicular liposomes are spherical and contain internal granular structures. The outer membrane is a lipid bilayer and the internal region contains small compartments separated by bilayer septum. Still yet another type of liposomes are oligolamellar vesicles (“OLVs”), which have a large center compartment surrounded by several peripheral lipid layers. These vesicles, having a diameter of 2-15 μm, are described in Callo, et al., Cryobiology 22(3):251-267 (1985).

Mezei, et al., U.S. Pat. Nos. 4,485,054 and 4,761,288 also describe methods of preparing lipid vesicles. More recently, Hsu, U.S. Pat. No. 5,653,996 describes a method of preparing liposomes utilizing aerosolization and Yiournas, et al., U.S. Pat. No. 5,013,497 describes a method for preparing liposomes utilizing a high velocity-shear mixing chamber. Methods are also described that use specific starting materials to produce ULVs (Wallach, et al., U.S. Pat. No. 4,853,228) or OLVs (Wallach, U.S. Pat. Nos. 5,474,848 and 5,628,936).

A comprehensive review of all the aforementioned lipid vesicles and methods for their preparation are described in “Liposome Technology”, ed. G. Gregoriadis, CRC Press Inc., Boca Raton, Fla., Vol. I, II & III (1984). This and the aforementioned references describing various lipid vesicles suitable for use with the disclosed compositions and methods are incorporated herein by reference.

Fatty acids (i.e., lipids) that can be conjugated to the provided compositions include those that allow the efficient incorporation of the proprotein convertase inhibitors into liposomes. Generally, the fatty acid is a polar lipid. Thus, the fatty acid can be a phospholipid The provided compositions can comprise either natural or synthetic phospholipid. The phospholipids can be selected from phospholipids containing saturated or unsaturated mono or disubstituted fatty acids and combinations thereof. These phospholipids can be dioleoylphosphatidylcholine, dioleoylphosphatidylserine, dioleoylphosphatidylethanolamine, dioleoylphosphatidylglycerol, dioleoylphosphatidic acid, palmitoyloleoylphosphatidylcholine, palmitoyloleoylphosphatidylserine, palmitoyloleoylphosphatidylethanolamine, palmitoyloleoylphophatidylglycerol, palmitoyloleoylphosphatidic acid, palmitelaidoyloleoylphosphatidylcholine, palmitelaidoyloleoylphosphatidylserine, palmitelaidoyloleoylphosphatidylethanolamine, palmitelaidoyloleoylphosphatidylglycerol, palmitelaidoyloleoylphosphatidic acid, myristoleoyloleoylphosphatidylcholine, myristoleoyloleoylphosphatidylserine, myristoleoyloleoylphosphatidylethanoamine, myristoleoyloleoylphosphatidylglycerol, myristoleoyloleoylphosphatidic acid, dilinoleoylphosphatidylcholine, dilinoleoylphosphatidylserine, dilinoleoylphosphatidylethanolamine, dilinoleoylphosphatidylglycerol, dilinoleoylphosphatidic acid, palmiticlinoleoylphosphatidylcholine, palmiticlinoleoylphosphatidylserine, palmiticlinoleoylphosphatidylethanolamine, palmiticlinoleoylphosphatidylglycerol, palmiticlinoleoylphosphatidic acid. These phospholipids may also be the monoacylated derivatives of phosphatidylcholine (lysophophatidylidylcholine), phosphatidylserine (lysophosphatidylserine), phosphatidylethanolamine (lysophosphatidylethanolamine), phophatidylglycerol (lysophosphatidylglycerol) and phosphatidic acid (lysophosphatidic acid). The monoacyl chain in these lysophosphatidyl derivatives may be palimtoyl, oleoyl, palmitoleoyl, linoleoyl myristoyl or myristoleoyl. The phospholipids can also be synthetic. Synthetic phospholipids are readily available commercially from various sources, such as AVANTI Polar Lipids (Albaster, Ala.); Sigma Chemical Company (St. Louis, Mo.). These synthetic compounds may be varied and may have variations in their fatty acid side chains not found in naturally occurring phospholipids. The fatty acid can have unsaturated fatty acid side chains with C14, C16, C18 or C20 chains length in either or both the PS or PC. Synthetic phospholipids can have dioleoyl (18:1)-PS; palmitoyl (16:0)-oleoyl (18:1)-PS, dimyristoyl (14:0)-PS; dipalmitoleoyl (16:1)-PC, dipalmitoyl (16:0)-PC, dioleoyl (18:1)-PC, palmitoyl (16:0)-oleoyl (18:1)-PC, and myristoyl (14:0)-oleoyl (18:1)-PC as constituents. Thus, as an example, the provided compositions can comprise palmitoyl 16:0.

iii. In Vivo/Ex Vivo

As described above, the compositions can be administered in a pharmaceutically acceptable carrier and can be delivered to the subject's cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

### B. METHODS

### 1. Modulating GSK3 Activity

Provided herein is a method of inhibiting GSK3 activity in a cell, comprising contacting the cell with a composition comprising FG01 protein, a nucleic acid encoding FG01, or a fragment thereof. Thus, provided herein is a method of inhibiting GSK3 activity in a cell, comprising contacting the cell with an isolated nucleic acid comprising a sequence at least about 70%, 75%, 80%, 85%, 90%, 92%, 92%, 93%, 94%, 95%, 96%, 97%, 98%, 99%, 100% or at least 70%, 75%, 80%, 85%, 90%, 92%, 92%, 93%, 94%, 95%, 96%, 97%, 98%, 99%, 100% identical to SEQ ID NO:1, or a fragment thereof at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, or 100 residues in length, operably linked to an expression control sequence, wherein the sequence encodes a polypeptide that binds adenylyl cyclase.

Also provided is a method of inhibiting GSK3 activity in a cell, comprising contacting the cell with a purified polypeptide comprising an amino acid sequence at least at least about 70%, 75%, 80%, 85%, 90%, 92%, 92%, 93%, 94%, 95%, 96%, 97%, 98%, 99%, 100% or at least 70%, 75%, 80%, 85%, 90%, 92%, 92%, 93%, 94%, 95%, 96%, 97%, 98%, 99%, 100% identical to the sequence SEQ ID NO:2, or a fragment thereof at least 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 85, 90, or 100 residues in length, wherein the polypeptide binds adenylyl cyclase.

### 2. Treatment

Provided herein are compositions and methods for treating a condition or disease affected by GSK3 levels or activity. The method can comprise administering a peptide comprising FG01 or a nucleic acid encoding FG01. Likewise, the method can comprise administering a peptidomimetic of FG01.

Thus, provided is a method of treating a subject with a condition or disease affected by GSK3 levels or activity, the method comprising administering to the subject an expression vector encoding protein FG01, such that a therapeutically effective amount of protein FG01, or a fragment thereof, is expressed in the subject.

Also provided is a method of treating a subject with a condition or disease affected by GSK3 levels or activity, the method comprising administering to the subject a cell comprising an expression vector encoding protein FG01, such that a therapeutically effective amount of protein FG01, or a fragment thereof, is expressed by the cell in the subject. In some aspects, the cell of the methods is a stem cell.

Also provided is a method of treating a subject with a condition or disease affected by GSK3 levels or activity, the method comprising administering to the subject a therapeutically effective amount of a composition comprising FG01 protein or a fragment thereof.

i. Neurodegenerative Disease

The condition or disease can in some aspects be a neurodegenerative disease. Thus, provided is a method of treating, preventing, or reducing the risk of developing a neurodegenerative disorder, such as Alzheimer's disease, in a subject comprising administering to the subject a therapeutically effective amount of a composition comprising FG01 or a nucleic acid encoding FG01, or a fragment thereof. Also provided is a method of treating a subject at risk for a neurodegenerative disorder, such as Alzheimer's disease, comprising administering to the subject a composition comprising FG01 or a nucleic acid encoding FG01, or a fragment thereof. As used herein, the terms “disorder” and “disease” are used interchangeably to refer to a condition in a subject.

As used herein, the term “Aβ-related disorder” or an “Aβ disorder” is a disease (e.g., Alzheimer's disease) or a condition (e.g., senile dementia) that involves an aberration or dysregulation of Aβ levels. An Aβ-related disorder includes, but is not limited to Alzheimer's disease, Down's syndrome and inclusion body myositis. Thus, the Aβ related disorder can be Alzheimer's disease. The progression of the Aβ related disorder can be slowed or reversed.

Also provided is a method for modulating amyloid-β peptide (Aβ) levels exhibited by a cell or tissue comprising contacting said cell or tissue with an amount of a composition comprising FG01 or a nucleic acid encoding FG01, or a fragment thereof, sufficient to modulate said Aβ levels.

As used herein, a cell or tissue may include, but not be limited to: an excitable cell, e.g., a sensory neuron, motomeuron, or interneuron; a glial cell; a primary culture of cells, e.g., a primary culture of neuronal or glial cells; cell(s) derived from a neuronal or glial cell line; dissociated cell(s); whole cell(s) or intact cell(s); permeabilized cell(s); a broken cell preparation; an isolated and/or purified cell preparation; a cellular extract or purified enzyme preparation; a tissue or organ, e.g., brain, brain structure, brain slice, spinal cord, spinal cord slice, central nervous system, peripheral nervous system, or nerve; tissue slices, and a whole animal. In certain embodiments, the brain structure is cerebral cortex, the hippocampus, or their anatomical and/or functional counterparts in other mammalian species. In certain embodiments, the cell or tissue is an N2a cell, a primary neuronal culture or a hippocampal tissue explant.

Also provided is a method for prevention, treatment, e.g., management, of an Aβ-related disorder, or amelioration of a symptom of an Aβ-related disorder such as Alzheimer's disease. It is understood that the methods described herein in the context of treating and/or ameliorating a symptom can also routinely be utilized as part of a prevention protocol.

Also provided is a method of treating, or ameliorating a symptom of, an Aβ-related disorder comprising administering to a subject in need of such treating or ameliorating an amount of a composition comprising FG01 or a nucleic acid encoding FG01, or a fragment thereof, sufficient to reduce Aβ levels in the subject such that the Aβ-related disorder is treated or a symptom of the AP related disorder is ameliorated.

Examples of neurodegenerative disorders include Alexander disease, Alper's disease, Alzheimer disease, Amyotrophic lateral sclerosis, Ataxia telangiectasia, Batten disease (also known as Spielmeyer-Vogt-Sjogren-Batten disease), Canavan disease, Cockayne syndrome, Corticobasal degeneration, Creutzfeldt-Jakob disease, Huntington disease, Kennedy's disease, Krabbe disease, Lewy body dementia, Machado-Joseph disease, Spinocerebellar ataxia type 3, Multiple sclerosis, Multiple System Atrophy, Parkinson's disease, Pelizaeus-Merzbacher Disease, Pick's disease, Primary lateral sclerosis, Refsum's disease, Sandhoff disease, Schilder's disease, Spielmeyer-Vogt-Sjogren-Batten disease (also known as Batten disease), Spinocerebellar ataxia (multiple types with varying characteristics), Spinal muscular atrophy, Steele-Richardson-Olszewski disease, Transmissible spongiform encephalopathies (TSE), and Tabes dorsalis.

The condition or disease can in some aspects be Alzheimer's disease. Alzheimer's disease is a progressive neurodegenerative disorder that is characterized by the formation of senile plaques and neurofibrillary tangles containing amyloid β (Aβ) peptide. These plaques are found in limbic and association cortices of the brain. The hippocampus is part of the limbic system and plays an important role in learning and memory. In subjects with Alzheimer's disease, accumulating plaques damage the neuronal architecture in limbic areas and eventually cripple the memory process.

Approximately twenty million people worldwide suffer with dementia that results from Alzheimer's disease. The disease can be early onset affecting individuals as young as 30 years of age, or it can be familial or sporadic. Familial Alzheimer's disease was once thought to be inherited strictly as an autosomal dominant trait; however, this view is changing as more genetic determinants are isolated. For example, some normal allelic variants of apolipoprotein E (ApoE), which is found in senile plaques, can either protect against or increase the risk of developing the disease (Strittmatter et al. (1993) Proc Natl Acad Sci 90:1977-1981).

Amyloid-β (Aβ) peptides are metabolites of the Alzheimer's disease-associated precursor protein, β-amyloid precursor protein (APP), and are believed to be the major pathological determinants of Alzheimer's disease (AD). These peptides consist mainly of 40 to 42 amino acids, Aβ1-40 (“Aβ40”) and Aβ1-42 (“Aβ42”), respectively. Aβ40 and Aβ42 are generated by two enzymatic cleavages occurring close to the C-terminus of APP. The enzymes responsible for the cleavage, β-secretase and γ-secretase, generate the - and C-termini of Aβ, respectively. The amino terminus of Aβ is formed by β-secretase cleavage between methionine residue 596 and aspartate residue 597 of APP (APP 695 isoform numbering) (see, e.g., U.S. Pat. No. 6,440,698; and U.S. Pat. No. 5,744,346).

γ-secretase activity cleaves at varying positions 38-, 40- or 43-residues C-terminal of this β-secretase cleavage to release Aβ peptides (see, e.g., U.S. Patent Application 20020025540). The complete molecular identity of γ-secretase enzyme is still unknown. Presenilin 1, or the closely related presenilin 2, is needed for γ-secretase activity. γ-secretase activity is reduced 80% in cultured cells derived from embryos genetically deleted for presenilin 1. All γ-secretase activity is lost in cells lacking both presenilin 1 and presenilin 2. Peptidomimetic inhibitors of γ-secretase activity can be crosslinked to presenilins 1 and 2, suggesting that these proteins are catalytic subunits for the cleavage. However, γ-secretase activity isolated from cells chromatographs as a large complex>1 M daltons. Recent genetic studies have identified three more proteins required for γ-secretase activity; nicastrin, aph-1 and pen-1. (Francis et al., 2002, Developmental Cell 3(1): 85-97; Steiner et al., 2002, J. Biol. Chemistry: 277(42): 39062-39065; and Li et al., 2002, J. Neurochem. 82(6): 1540-1548). Accumulation of presenilin into high molecular weight complexes is altered in cells lacking these proteins.

A third enzyme, α-secretase, cleaves the precursor protein between the β- and γ-cleavage sites, thus precluding AP production and releasing an approximately 3 kDa peptide known as P3, which is non-pathological. Both β- and α-secretase cleavage also result in soluble, secreted-terminal fragments of APP, known as sAPβ and sAPPα, respectively. The sAPPα fragment has been suggested to be neuroprotective.

In normal individuals, the Aβ peptide is found in two predominant forms, the majority Aβ-40 (also known as Aβ1-40) form and the minority Aβ42 (also known as Aβ1-42) form, each having a distinct COOH-terminus. The major histological lesions of AD are neuritic plaques and neurofibrillary tangles occurring in affected brain regions. Neuritic plaques consist of Aβ peptides, primarily Aβ40 and Aβ42. Although healthy neurons produce at least ten times more Aβ40 compared to Aβ42, plaques contain a larger proportion of the less soluble Aβ42. Patients with the most common form of familial Alzheimer's disease show an increase in the amount of the Aβ42 form. The Aβ40 form is not associated with early deposits of amyloid plaques. In contrast, the Aβ42 form accumulates early and predominantly in the parenchymal plaques and there is strong evidence that Aβ42 plays a major role in amyloid plaque deposits in familial Alzheimer's disease patients (Roher et al., 1993, Proc. Natl. Acad. Sci. USA 90:10836; Iwatasubo, T., et al., 1994 Neuron 13:45; Yamaguchi et al, 1995, Amyloid Int. J. Clin. Invest. 2:7-16; and Mann et al., 1996 Am. J. Pathol. 148:1257).

Mutations in four genes are known to predispose an individual to Alzheimer's disease: ApoE, amyloid precursor protein (APP), presenilin-1, and presenilin-2 (Selkoe (1999) Nature 399:A23-A31). The e4 allele of the ApoE gene confers increased risk for late onset Alzheimer's disease. β-amyloid protein (Aβ) is the major component of senile plaques, and it is normally formed when β- and γ-secretase cleave APP. In Alzheimer's disease patients, large quantities of Aβ are generated and accumulate extracellularly in these neuropathological plaques. Efforts to understand the mechanism underlying Aβ deposition have recently focused on the APP-cleaving secretase. In fact, two yeast aspartyl proteases have been shown to process APP in vitro (Zhang et al. (1997) Biochem Biophys Acta 1359:110-122). Evidence using peptidomimetic probes further confirms that the secretase is an intramembrane-cleaving aspartyl protease (Wolfe et al. (1999) Biochemistry 38:4720-4727). The presenilin-1 gene is a candidate for the γ-secretase that cleaves the APP carboxyl terminus. Several lines of evidence support the involvement of presenilins in the disease process. Presenilin can be coimmunoprecipitated with APP, and mutations in the presenilin genes increase production of the 42-amino acid peptide form of Aβ. These missense point mutations result in a particularly aggressive, early onset form of the disease (Haaas and DeStrooper (1999) Science 286:916-919).

The proteases, BACE1 and BACE2 (β-site APP cleaving enzymes 1 and 2) which appear to be β-secretase, are potential therapeutic targets because of their ability to cleave APP. Vassar et al. (1999; Science 286:735-741) have found that BACE1 is an aspartyl protease with β-secretase activity which cleaves APP to produce Aβ peptide in vitro. It is expressed at moderate levels across all brain regions and is concentrated in neurons but not in glia. BACE2, which has 52% amino acid identity with BACE1, has been described by Saunders et al. (1999; Science 286:1255a). Whereas BACE1 maps to the long arm of chromosome 11, BACE2 maps to the Down syndrome region of chromosome 21 (Acquati et al. (2000) 468: 59-64; Saunders et al. supra). This location is significant because middle-aged Down syndrome patients have enhanced β-amyloid deposits. Other members of the BACE family can also participate in this APP cleavage: the amino terminals of Aβ peptides appear to be cleaved heterogeneously indicating that there can be several β-secretase involved in APP processing (Vassar (1999) Science 286:735-741).

Associations between Alzheimer's disease and many other genes and proteins have been reported. Fetal Alzheimer antigen (FALZ) and synuclein a (SNCA) are found in brain plaques and tangles. Inheritance of some gene polymorphisms is also linked to increased risk of developing the disease. For example, a polymorphism in the gene encoding β2-macroglobulin, a protein that can act as a protease inhibitor, is associated with increased risk for developing a late-onset form of Alzheimer's disease.

One hundred years ago Alois Alzheimer described the major behavioral and neuropathological features of the neurodegenerative disorder bearing his name. AD is characterized clinically/behaviorally by progressive impairment of memory and cognition. Neuropathological and neurobiological changes associated with this slow progression of clinical symptoms include accumulation of amyloid plaques and neurofibrillary tangles (NFTs) (Gearing M. et al., The Consortium to Establish a Registry for Alzheimer's Disease (CERAD). Part X. Neuropathology confirmation of the clinical diagnosis of Alzheimer's disease. Neurology. 1995; 45(3 Pt 1):461-466) gliosis (Unger J W., Microscopy Res. Technique. 1998; 43:24-28), reduced dendritic plasticity relative to normal aged (Buell S J. Coleman P D., Science. 1979; 206(4420):854-856), Flood D G. et al., Brain Research. 1985; 345(2):366-368, Flood D G., et al., Brain Research. 1987; 402(2):205-216), and reduced density of neurons (Coleman P D. Flood D G., et al., Neurobiology of Aging. 1987; 8(6):521-545), Terry R D, et al., 1987; 21:530-539, West M J, et al., Lancet. 1994; 344:769-772) and synapses (Scheff S W. et al., Neurobiology of Aging. 1990; 11(1):29-37).

Studies of altered gene expression in Alzheimer's disease brain tissue have shown a general reduction of message level estimated at about 35% (Doebler J A, et al., Journal of Neuropathology & Experimental Neurology. 1987; 46(1):28-39), (Griffin W S, et al., Alzheimer Disease & Associated Disorders. 1990; 4(2):69-78), (Harrison P J, et al., Psychological Medicine. 1991; 21:855-866). Against this background of a general reduction of mRNA, selected studies have demonstrated increased as well as decreased expression of a wide variety of genes. Some gene classes affected in Alzheimer's disease are expressed in a neuron specific manner. These especially include decreased expression of selected genes that are related to synaptic structure and function and the neuronal cytoskeleton (Ginsberg S D. et al., Annals of Neurology. 2000; 48(1):77-87), (Yao P, et al., Journal of Neuroscience. 1998; 18(7):2399-2411). Other classes of genes whose expression is altered in AD include those related to the cell cycle (Arendt T., Neurobiology of Aging. 2000; 21(6):783-796), (Husseman J W., et al., Neurobiology of Aging. 2000; 21(6):815-828), (Nagy Z., et al., Neurobiology of Aging. 2000; 21(6):761-769), (Vincent I, et al., J. Neurosci. 1997; 17:3588-3598) and inflammatory/stress responses (for a review, see (Akiyama H., et al., Neurobiology of Aging. 2000; 21(3):383-421)). These gene classes are expressed in a variety of cell types that reside outside the nervous system including leukocytes (Wakutani Y. et al., Dementia. 1995; 6(6):301-305), monocytes (Jung S S. et al., Neurobiology of Aging. 1999; 20(3):249-257), and epithelial cells (Schmitz A., et al., Histochemistry & Cell Biology. 2002; 117(2):171-180) as well as other cell types.

Multivariate analysis of profiles of expression of multiple gene products (messages) by single neurons or homogenates from postmortem human brain can be used to distinguish Alzheimer's disease from control samples (Cheetham J E., et al., Journal of Neuroscience Methods. 1997; 77(1):43-48, Chow, N., et al., Proc. Natl. Acad. Sci. USA. 1998; 95:9620-9625).

Symptoms of Aβ-related disorders are well known to those of skill in the art. For example, symptoms of Alzheimer's disease are well known in the art and can include, e.g., memory loss, mild cognitive impairment, cognitive decline, severe cognitive impairment and personality changes that result in loss of functional ability, e.g., over the course of a decade. In debilitated states, patients usually exhibit severe impairment, and retain only vegetative neurologic function. Symptoms of Alzheimer's disease can also include certain art-known neuropathological lesions, including intracellular neurofibrillary tangles and extracellular parenchymal and cerebrovascular amyloid.

Thus, also provided is a method of reducing amyloid beta (Aβ) generation in a subject, comprising administering to the subject a vector comprising an isolated nucleic acid comprising a sequence at least at least about 70%, 75%, 80%, 85%, 90%, 92%, 92%, 93%, 94%, 95%, 96%, 97%, 98%, 99%, 100% or at least 70%, 75%, 80%, 85%, 90%, 92%, 92%, 93%, 94%, 95%, 96%, 97%, 98%, 99%, 100% identical to SEQ ID NO:1, or a fragment thereof at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, or 100 residues in length, operably linked to an expression control sequence, wherein the sequence encodes a polypeptide that binds adenylyl cyclase.

Also provided is a method of treating or preventing Alzheimer's disease in a subject, comprising administering to the subject a vector comprising an isolated nucleic acid comprising a sequence at least at least about 70%, 75%, 80%, 85%, 90%, 92%, 92%, 93%, 94%, 95%, 96%, 97%, 98%, 99%, 100% or at least 70%, 75%, 80%, 85%, 90%, 92%, 92%, 93%, 94%, 95%, 96%, 97%, 98%, 99%, 100% identical to SEQ ID NO:1, or a fragment thereof at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, or 100 residues in length, operably linked to an expression control sequence, wherein the sequence encodes a polypeptide that binds adenylyl cyclase.

Also provided is a method of reducing amyloid beta (Aβ) generation in a subject, comprising administering to the subject a purified polypeptide comprising an amino acid sequence at least at least about 70%, 75%, 80%, 85%, 90%, 92%, 92%, 93%, 94%, 95%, 96%, 97%, 98%, 99%, 100% or at least 70%, 75%, 80%, 85%, 90%, 92%, 92%, 93%, 94%, 95%, 96%, 97%, 98%, 99%, 100% identical to the sequence SEQ ID NO:2, or a fragment thereof at least 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 85, 90, or 100 residues in length, wherein the polypeptide binds adenylyl cyclase.

Also provided is a method of treating or preventing Alzheimer's disease in a subject, comprising administering to the subject a purified polypeptide comprising an amino acid sequence at least at least about 70%, 75%, 80%, 85%, 90%, 92%, 92%, 93%, 94%, 95%, 96%, 97%, 98%, 99%, 100% or at least 70%, 75%, 80%, 85%, 90%, 92%, 92%, 93%, 94%, 95%, 96%, 97%, 98%, 99%, 100% identical to the sequence SEQ ID NO:2, or a fragment thereof at least 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 85, 90, or 100 residues in length, wherein the polypeptide binds adenylyl cyclase.

ii. Cancer

The condition or disease can in some aspects be cancer. The cancer of the disclosed methods can be any cell in a subject undergoing unregulated growth, invasion, or metastasis. In some aspects, the cancer can be any neoplasm or tumor for which GSK3 is known to be involved. Thus, the cancer can be a sarcoma, lymphoma, leukemia, carcinoma, blastoma, or germ cell tumor. A representative but non-limiting list of cancers that the disclosed compositions can be used to treat include lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, and pancreatic cancer.

Thus, provided is a method of treating or preventing cancer in a subject, comprising administering to the subject a composition comprising FG01 or a nucleic acid encoding FG01, or a fragment thereof.

Thus, provided is a method of treating or preventing cancer in a subject, comprising administering to the subject a vector comprising an isolated nucleic acid comprising a sequence at least at least about 70%, 75%, 80%, 85%, 90%, 92%, 92%, 93%, 94%, 95%, 96%, 97%, 98%, 99%, 100% or at least 70%, 75%, 80%, 85%, 90%, 92%, 92%, 93%, 94%, 95%, 96%, 97%, 98%, 99%, 100% identical to SEQ ID NO:1, or a fragment thereof at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, or 100 residues in length, operably linked to an expression control sequence, wherein the sequence encodes a polypeptide that binds adenylyl cyclase.

Thus, provided is a method of treating or preventing cancer in a subject, comprising administering to the subject a purified polypeptide comprising an amino acid sequence at least at least about 70%, 75%, 80%, 85%, 90%, 92%, 92%, 93%, 94%, 95%, 96%, 97%, 98%, 99%, 100% or at least 70%, 75%, 80%, 85%, 90%, 92%, 92%, 93%, 94%, 95%, 96%, 97%, 98%, 99%, 100% identical to the sequence SEQ ID NO:2, or a fragment thereof at least 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 85, 90, or 100 residues in length, wherein the polypeptide binds adenylyl cyclase.

iii. Autoimmune Disease

The condition or disease can in some aspects be an autoimmune disease. Autoimmune disease refers to diseases including, but not limited to, rheumatoid arthritis, multiple sclerosis, diabetes, inflammatory bowel disease, psoriasis, systemic lupus erythematosus, allergic disease, or asthma.

iv. Metabolic Disease

The condition or disease can in some aspects be a metabolic disease. Metabolic disease refers to diabetes and disorders of carbohydrate metabolism, amino acid metabolism, organic acid metabolism (organic acidurias), fatty acid oxidation and mitochondrial metabolism, porphyrin metabolism, purine or pyrimidine metabolism, steroid metabolism, mitochondrial function, peroxisomal function, Lysosomal storage disorders, Acromegaly, Addison's Disease, Cushing's Syndrome, Cystic Fibrosis, Endocrine Diseases, Human Growth Hormone related diseases, Hyperparathyroidism, Multiple Endocrine Neoplasia Type 1, Prolactinoma, Turner Syndrome.

Thus, the condition or disease can in some aspects be diabetes. The World Health Organization recognizes three main forms of diabetes: type 1, type 2, and gestational diabetes (occurring during pregnancy), which have similar signs, symptoms, and consequences, but different causes and population distributions. Type 1 is usually due to autoimmune destruction of the pancreatic beta cells which produce insulin. Type 2 is characterized by tissue-wide insulin resistance and varies widely; it sometimes progresses to loss of beta cell function. Gestational diabetes is similar to type 2 diabetes, in that it involves insulin resistance. The hormones of pregnancy cause insulin resistance in those women genetically predisposed to developing this condition. Types 1 and 2 are incurable chronic conditions, but have been treatable since insulin became medically available in 1921. Gestational diabetes typically resolves with delivery. Thus, in some aspects of the disclosed method, the subject has been diagnosed with type 1 or type 2 diabetes mellitus or gestational diabetes.

Diabetes can cause many complications. Acute glucose level abnormalities may occur if insulin level is not well-controlled. Serious long-term complications include cardiovascular disease (doubled risk), chronic renal failure (the main cause of dialysis in developed world adults), retinal damage (which can lead to blindness and is the most significant cause of adult blindness in the non-elderly in the developed world), nerve damage (of several kinds), and microvascular damage, which may cause erectile dysfunction (impotence) and poor healing. Poor healing of wounds, particularly of the feet, can lead to gangrene which can require amputation—the leading cause of non-traumatic amputation in adults in the developed world.

Diabetes, without qualification, usually refers to diabetes mellitus, but there are several rarer conditions also named diabetes. The most common of these is diabetes insipidus (unquenchable diabetes) in which the urine is not sweet; it can be caused by either kidney (nephrogenic DI) or pituitary gland (central DI) damage.

There are several rare causes of diabetes mellitus that do not fit into type 1, type 2, or gestational diabetes, namely genetic defects in beta cells (autosomal or mitochondrial), genetically-related insulin resistance, with or without lipodystrophy (abnormal body fat deposition), diseases of the pancreas (e.g. chronic pancreatitis, cystic fibrosis), hormonal defects, and chemicals or drugs. In addition, the tenth version of the International Statistical Classification of Diseases (ICD-10) contained a diagnostic entity named “malnutrition-related diabetes mellitus” (MRDM or MMDM, ICD-10 code E12).

The classical triad of diabetes symptoms is polyuria (frequent urination), polydipsia (increased thirst and consequent increased fluid intake) and polyphagia (increased appetite). These symptoms may develop quite fast in type 1, particularly in children (weeks or months) but may be subtle or completely absent—as well as developing much more slowly—in type 2. In type 1 there may also be weight loss (despite normal or increased eating) and irreducible fatigue. These symptoms may also manifest in type 2 diabetes in patients whose diabetes is poorly controlled.

Diabetes mellitus is characterized by recurrent or persistent hyperglycemia, and is diagnosed by demonstrating any one of the following:

fasting plasma glucose level at or above 126 mg/dL (7.0 mmol/l);

plasma glucose at or above 200 mg/dL or 11.1 mmol/l two hours after a 75 g oral glucose load as in a glucose tolerance test;

random plasma glucose at or above 200 mg/dL or 11.1 mmol/l.

Patients with fasting sugars between 6.1 and 7.0 mmol/l (ie, 110 and 125 mg/dL) are considered to have “impaired fasting glucose” and patients with plasma glucose at or above 140 mg/dL or 7.8 mmol/l two hours after a 75 g oral glucose load are considered to have “impaired glucose tolerance.” “Prediabetes” is either impaired fasting glucose or impaired glucose tolerance; the latter in particular is a major risk factor for progression to full-blown diabetes mellitus as well as cardiovascular disease. Thus, in some aspects, the subject has been diagnosed with pre-diabetes.

While not generally used for diagnosis, an elevated level of glucose irreversibly bound to hemoglobin (termed glycosylated hemoglobin, Hb_{A1c}, or A1C) of 6.0% or higher (the 2003 revised U.S. standard) is considered abnormal. HbA1c is primarily used as a treatment-tracking test reflecting average blood glucose levels over the preceding 90 days (approximately). However, some physicians may order this test at the time of diagnosis to track changes over time. The current recommended goal for HbA1c in patients with diabetes is <7.0%, which as defined as “good glycemic control”, although some guidelines are stricter (<6.5%). People with diabetes who have HbA1c levels within this range have a significantly lower incidence of complications from diabetes, including retinopathy and diabetic nephropathy.

Thus, in some aspects, the subject has been diagnosed with diabetes or pre-diabetes. Thus, in some aspects, the subject has a fasting plasma glucose level of at least 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, or 140 mg/dL. Thus, in some aspects, the subject has a plasma glucose of at least 140, 150, 160, 170, 180, 190, 200, 210, 220, 230, 240, 250, 260, 270, 290 or 300 mg/dL two hours after a 75 g oral glucose load in a glucose tolerance test. Thus, in some aspects, the subject has a random plasma glucose of at least 140, 150, 160, 170, 180, 190, 200, 210, 220, 230, 240, 250, 260, 270, 290 or 300 mg/dL. Thus, in some aspects, the subject has a hemoglobin HbA1C (A1C) level greater than 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or 9.0 percent.

In other aspects, the subject does not have clinical indications of diabetes. Thus, in some aspects, the subject has a fasting plasma glucose level of less than 126, 125, 124, 123, 121, 120, 115, 110, 105, 100, 95, 90, 85, or 80 mg/dL. Thus, in some aspects, the subject has a plasma glucose of less than 200, 195, 190, 185, 180, 175, 170, 165, 160, 155, 150, 145, 140, 135, or 130 mg/dL two hours after a 75 g oral glucose load in a glucose tolerance test. Thus, in some aspects, the subject has a random plasma glucose of at less than 200, 195, 190, 185, 180, 175, 170, 165, 160, 155, 150, 145, 140, 135, or 130 mg/dL. Thus, in some aspects, the subject has a hemoglobin Hb_{A1C} (A1C) level less than 6.0, 5.9, 5.8, 5.7, 5.6, 5.5, 5.4, 5.3, 5.2, 5.1, 5.0, 4.9, 4.8, 4.7, 4.6, 4.5, 4.4, 4.3, 4.2, 4.1, or 4.0 percent.

Chronic elevation of blood glucose level leads to damage of blood vessels. In diabetes, the resulting problems are grouped under “microvascular disease” (due to damage to small blood vessels) and “macrovascular disease” (due to damage to the arteries). The damage to small blood vessels leads to a microangiopathy, which can cause diabetic retinopathy and/or diabetic nephropathy. Angiopathy means disease of the blood vessels (arteries, veins, and capillaries). In microangiopathy, the walls of very small blood vessels (capillaries) become so thick and weak that they bleed, leak protein, and slow the flow of blood. For example, diabetics can develop microangiopathy with thickening of capillaries in many areas including the eye.

Diabetic retinopathy refers to growth of friable and poor-quality new blood vessels in the retina as well as macular edema (swelling of the macula), which can lead to severe vision loss or blindness. Retinal damage (e.g., from microangiopathy) makes it the most common cause of blindness among non-elderly adults in the US.

Diabetic nephropathy refers to damage to the kidney which can lead to chronic renal failure, eventually requiring dialysis. Diabetes mellitus is the most common cause of adult kidney failure worldwide in the developed world.

Thus, provided is a method of treating or preventing diabetes in a subject, comprising administering to the subject a composition comprising FG01 or a nucleic acid encoding FG01, or a fragment thereof.

Also provided is a method of treating or preventing diabetes in a subject, comprising administering to the subject a vector comprising an isolated nucleic acid comprising a sequence at least at least about 70%, 75%, 80%, 85%, 90%, 92%, 92%, 93%, 94%, 95%, 96%, 97%, 98%, 99%, 100% or at least 70%, 75%, 80%, 85%, 90%, 92%, 92%, 93%, 94%, 95%, 96%, 97%, 98%, 99%, 100% identical to SEQ ID NO:1, or a fragment thereof at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, or 100 residues in length, operably linked to an expression control sequence, wherein the sequence encodes a polypeptide that binds adenylyl cyclase.

Also provided is a method of treating or preventing diabetes in a subject, comprising administering to the subject a purified polypeptide comprising an amino acid sequence at least at least about 70%, 75%, 80%, 85%, 90%, 92%, 92%, 93%, 94%, 95%, 96%, 97%, 98%, 99%, 100% or at least 70%, 75%, 80%, 85%, 90%, 92%, 92%, 93%, 94%, 95%, 96%, 97%, 98%, 99%, 100% identical to the sequence SEQ ID NO:2, or a fragment thereof at least 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 85, 90, or 100 residues in length, wherein the polypeptide binds adenylyl cyclase.

As further disclosed herein, any of the herein disclosed treatment methods can further comprise monitoring measuring GSK3 levels or activity in the subject. As further disclosed herein, any of the herein disclosed treatment methods can further comprise measuring APP levels in the subject. As further disclosed herein, any of the herein disclosed treatment methods can further comprise measuring Aβ levels in the subject. As further disclosed herein, any of the herein disclosed treatment methods can further comprise monitoring amyloid plaques in the subject.

### 3. Diagnosis

Also provided herein are compositions and methods for diagnosing or monitoring a condition or disease affected by GSK3 levels or activity. The method can comprise measuring levels of human homologue of FG01 or a nucleic acid encoding the human homologue of FG01, in the subject, wherein lower than normal levels of FG01 is an indication that the subject has a condition or disease affected by GSK3 levels or activity. The human homologue of FG01 can comprise an amino acid sequence having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to SEQ ID NO:2.

### 4. Screening Assays

Also provided is a method of identifying a modulator of glycogen synthase kinase-3 (GSK-3), the method comprising contacting purified polypeptide comprising an amino acid sequence at least at least about 70%, 75%, 80%, 85%, 90%, 92%, 92%, 93%, 94%, 95%, 96%, 97%, 98%, 99%, 100% or at least 70%, 75%, 80%, 85%, 90%, 92%, 92%, 93%, 94%, 95%, 96%, 97%, 98%, 99%, 100% identical to the sequence SEQ ID NO:2, or a fragment thereof at least 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 85, 90, or 100 residues in length, wherein the polypeptide binds adenylyl cyclase, with a candidate agent (test compound); and determining whether the candidate agents binds to the purified polypeptide, said binding being an indication that the candidate agents is a modulator of glycogen synthase kinase-3 (GSK-3).

Candidate agents encompass numerous chemical classes, but are most often organic molecules, e.g., small organic compounds having a molecular weight of more than 100 and less than about 2,500 daltons. Candidate agents comprise functional groups necessary for structural interaction with proteins, particularly hydrogen bonding, and typically include at least an amine, carbonyl, hydroxyl or carboxyl group, for example, at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. In a further embodiment, candidate agents are peptides.

In general, candidate agents can be identified from large libraries of natural products or synthetic (or semi-synthetic) extracts or chemical libraries according to methods known in the art. Those skilled in the field of drug discovery and development will understand that the precise source of test extracts or compounds is not critical to the disclosed screening procedure(s). Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, polypeptide- and nucleic acid-based compounds. Synthetic compound libraries are commercially available, e.g., from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods. In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their effect on the pathways and diseases disclosed herein.

In some embodiments, the candidate agents are proteins. In some aspects, the candidate agents are naturally occurring proteins or fragments of naturally occurring proteins. Thus, for example, cellular extracts containing proteins, or random or directed digests of proteinaceous cellular extracts, can be used. In this way libraries of procaryotic and eucaryotic proteins can be made for screening using the methods herein. The libraries can be bacterial, fungal, viral, and vertebrate proteins, and human proteins.

When a crude extract is found to have a desired activity, further fractionation of the positive lead extract is necessary to isolate chemical constituents responsible for the observed effect. Thus, the goal of the extraction, fractionation, and purification process is the careful characterization and identification of a chemical entity within the crude extract having an activity disclosed herein. The same assays described herein for the detection of activities in mixtures of compounds can be used to purify the active component and to test derivatives thereof. Methods of fractionation and purification of such heterogenous extracts are known in the art. If desired, compounds shown to be useful agents for treatment are chemically modified according to methods known in the art. Compounds identified as being of therapeutic value may be subsequently analyzed using animal models for diseases or conditions in which it is desirable to regulate or mimic activity of FG01.

Also provided is a method of identifying a modulator of glycogen synthase kinase-3 (GSK-3), the method comprising providing a cell comprising a nucleic acid encoding FG01 operably linked to an expression control sequence, wherein the nucleic acid comprises a sequence at least at least about 70%, 75%, 80%, 85%, 90%, 92%, 92%, 93%, 94%, 95%, 96%, 97%, 98%, 99%, 100% or at least 70%, 75%, 80%, 85%, 90%, 92%, 92%, 93%, 94%, 95%, 96%, 97%, 98%, 99%, 100% identical to SEQ ID NO:1, or a fragment thereof at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, or 100 residues in length, wherein the sequence encodes a polypeptide that binds adenylyl cyclase, contacting the cell with a candidate agent (test compound); and measuring expression of FG01, an increase or decrease in FG01 expression an indication that the candidate agent is a modulator of GSK-3.

Also provided is a process for making a modulator of glycogen synthase kinase-3 (GSK-3), the method comprising manufacturing the compound identified by the method disclosed herein.

Also provided is a method of identifying a compound that binds to FG01, the method comprising providing a cell expressing FG01; contacting the cell with a candidate agent (test compound); and determining whether the candidate agent binds to FG01.

### 5. Administration

The disclosed compounds and compositions can be administered in any suitable manner. The manner of administration can be chosen based on, for example, whether local or systemic treatment is desired, and on the area to be treated. For example, the compositions can be administered orally, parenterally (e.g., intravenous, subcutaneous, intraperitoneal, or intramuscular injection), by inhalation, extracorporeally, topically (including transdermally, ophthalmically, vaginally, rectally, intranasally) or the like.

As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The exact amount of the compositions required can vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein. Thus, effective dosages and schedules for administering the compositions may be determined empirically, and making such determinations is within the skill in the art. The dosage ranges for the administration of the compositions are those large enough to produce the desired effect in which the symptoms disorder are effected. The dosage generally should not be so large as to cause adverse side effects, such as unwanted cross-reactions, anaphylactic reactions, and the like. Generally, the dosage can vary with the age, condition, sex and extent of the disease in the patient, route of administration, or whether other drugs are included in the regimen, and can be determined by one of skill in the art. The dosage can be adjusted by the individual physician in the event of any counter indications. Dosage can vary, and can be administered in one or more dose administrations daily, for one or several days. Guidance can be found in the literature for appropriate dosages for given classes of pharmaceutical products.

For example, a typical daily dosage of a composition comprising FG01, a nucleic acid encoding FG01, or a fragment thereof, used alone might range from about 1 μg/kg to up to 100 mg/kg of body weight or more per day, depending on the factors mentioned above.

Following administration of a disclosed composition, the efficacy of the therapeutic peptide or nucleic acid can be assessed in various ways well known to the skilled practitioner. For instance, one of ordinary skill in the art will understand that a composition disclosed herein is efficacious in treating or inhibiting Alzheimer's disease in a subject by observing that the composition reduces amyloid beta or prevents a further increase in plaque formation. Amyloid beta, GSK3, and other indicators of therapeutic efficacy disclosed herein can be measured by methods that are known in the art, for example, using polymerase chain reaction assays to detect the presence of nucleic acid or antibody assays to detect the presence of protein in a sample (e.g., but not limited to, blood) from a subject or patient, or by measuring the level of circulating levels in the patient. Efficacy of the administration of the disclosed composition may also be determined by routine diagnostic means. For example, efficacy of the disclosed compositions for treating diabetes can be determined by monitoring blood sugar.

The compositions disclosed herein may be administered prophylactically to patients or subjects who are at risk for neurodegenerative disease, cancer, or diabetes or who have been newly diagnosed with neurodegenerative disease, cancer, or diabetes.

The disclosed compositions and methods can also be used for example as tools to isolate and test new drug candidates for a variety of neurodegenerative disease, cancer, or diabetes related diseases.

### C. KITS

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for modulating GSK3 activity, the kit comprising FG01 or a nucleic acid encoding FG01. The kits also can contain a means for detecting GSK3 activity.

### D. USES

The disclosed compositions can be used in a variety of ways as research tools. Other uses are disclosed, apparent from the disclosure, and/or will be understood by those in the art.

### E. METHODS OF MAKING THE COMPOSITIONS

The compositions disclosed herein and the compositions necessary to perform the disclosed methods can be made using any method known to those of skill in the art for that particular reagent or compound unless otherwise specifically noted.

1. Nucleic Acid Synthesis

For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

2. Peptide Synthesis

One method of producing the disclosed proteins, such as SEQ ID NO:2, is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant G A (1992) Synthetic Peptides: A User Guide. W.H. Freeman and Co., N.Y. (1992); Bodansky M and Trost B., Ed. (1993) Principles of Peptide Synthesis. Springer-Verlag Inc., NY (which is herein incorporated by reference at least for material related to peptide synthesis). Alternatively, the peptide or polypeptide is independently synthesized in vivo as described herein. Once isolated, these independent peptides or polypeptides may be linked to form a peptide or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide—thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

3. Processes for Making the Compositions

Disclosed are processes for making the compositions as well as making the intermediates leading to the compositions. For example, disclosed are nucleic acids in SEQ ID NO:1. There are a variety of methods that can be used for making these compositions, such as synthetic chemical methods and standard molecular biology methods. It is understood that the methods of making these and the other disclosed compositions are specifically disclosed.

Disclosed are nucleic acid molecules produced by the process comprising linking in an operative way a nucleic acid comprising the sequence set forth in SEQ ID NO:1 and a sequence controlling the expression of the nucleic acid.

Also disclosed are nucleic acid molecules produced by the process comprising linking in an operative way a nucleic acid molecule comprising a sequence having 80% identity to a sequence set forth in SEQ ID NO:1, and a sequence controlling the expression of the nucleic acid.

Disclosed are nucleic acid molecules produced by the process comprising linking in an operative way a nucleic acid molecule comprising a sequence that hybridizes under stringent hybridization conditions to a sequence set forth SEQ ID NO:1 and a sequence controlling the expression of the nucleic acid.

Disclosed are nucleic acid molecules produced by the process comprising linking in an operative way a nucleic acid molecule comprising a sequence encoding a peptide set forth in SEQ ID NO:2 and a sequence controlling an expression of the nucleic acid molecule.

Disclosed are nucleic acid molecules produced by the process comprising linking in an operative way a nucleic acid molecule comprising a sequence encoding a peptide having 80% identity to a peptide set forth in SEQ ID NO:2 and a sequence controlling an expression of the nucleic acid molecule.

Disclosed are nucleic acids produced by the process comprising linking in an operative way a nucleic acid molecule comprising a sequence encoding a peptide having 80% identity to a peptide set forth in SEQ ID NO:2, wherein any change is a conservative change, and a sequence controlling an expression of the nucleic acid molecule.

Disclosed are cells produced by the process of transforming the cell with any of the disclosed nucleic acids. Disclosed are cells produced by the process of transforming the cell with any of the non-naturally occurring disclosed nucleic acids.

Disclosed are any of the disclosed peptides produced by the process of expressing any of the disclosed nucleic acids. Disclosed are any of the non-naturally occurring disclosed peptides produced by the process of expressing any of the disclosed nucleic acids. Disclosed are any of the disclosed peptides produced by the process of expressing any of the non-naturally disclosed nucleic acids.

Disclosed are animals produced by the process of transfecting a cell within the animal with any of the nucleic acid molecules disclosed herein. Disclosed are animals produced by the process of transfecting a cell within the animal any of the nucleic acid molecules disclosed herein, wherein the animal is a mammal. Also disclosed are animals produced by the process of transfecting a cell within the animal any of the nucleic acid molecules disclosed herein, wherein the mammal is mouse, rat, rabbit, cow, sheep, pig, or primate.

Also disclose are animals produced by the process of adding to the animal any of the cells disclosed herein.

### F. DEFINITIONS

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents.

It must be noted that as used herein and in the appended claims, the singular forms “a,” “an,” and “the” include plural reference unless the context clearly dictates otherwise. Thus, for example, reference to “a peptide” includes a plurality of such peptides, reference to “the peptide” is a reference to one or more peptides and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Throughout the description and claims of this specification, the word “comprise” and variations of the word, such as “comprising” and “comprises,” means “including but not limited to,” and is not intended to exclude, for example, other additives, components, integers or steps.

As used herein, the term “subject” means any target of administration. The subject can be a vertebrate, for example, a mammal. Thus, the subject can be a human. The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

“Activities” of a protein include, for example, transcription, translation, intracellular translocation, secretion, phosphorylation by kinases, cleavage by proteases, homophilic and heterophilic binding to other proteins, ubiquitination.

“Promote,” “promotion,” and “promoting” refer to an increase in an activity, response, condition, disease, or other biological parameter. This can include but is not limited to the initiation of the activity, response, condition, or disease. This may also include, for example, a 10% increase in the activity, response, condition, or disease as compared to the native or control level. Thus, the reduction can be a 10, 20, 30, 40, 50, 60, 70, 80, 90, 100%, or any amount of increase in between as compared to native or control levels.

By “treatment” is meant the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “therapeutically effective” means that the amount of the composition used is of sufficient quantity to ameliorate one or more causes or symptoms of a disease or disorder. Such amelioration only requires a reduction or alteration, not necessarily elimination.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### G. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### Generation of RHKO Cell Libraries

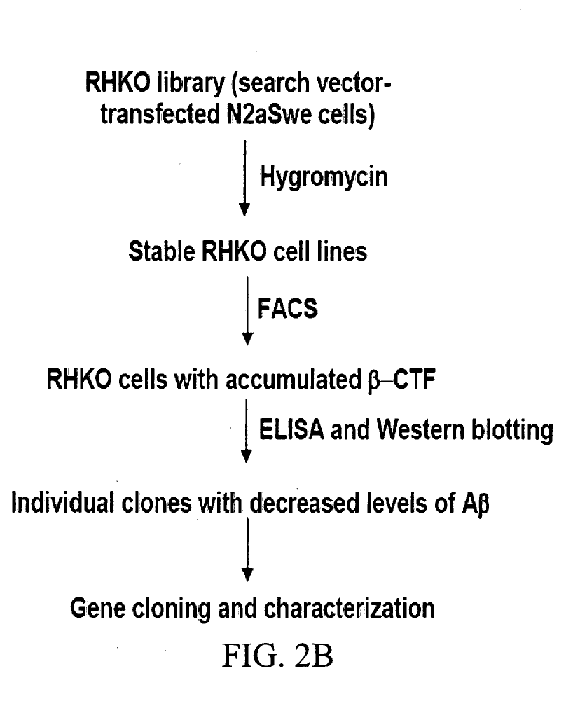

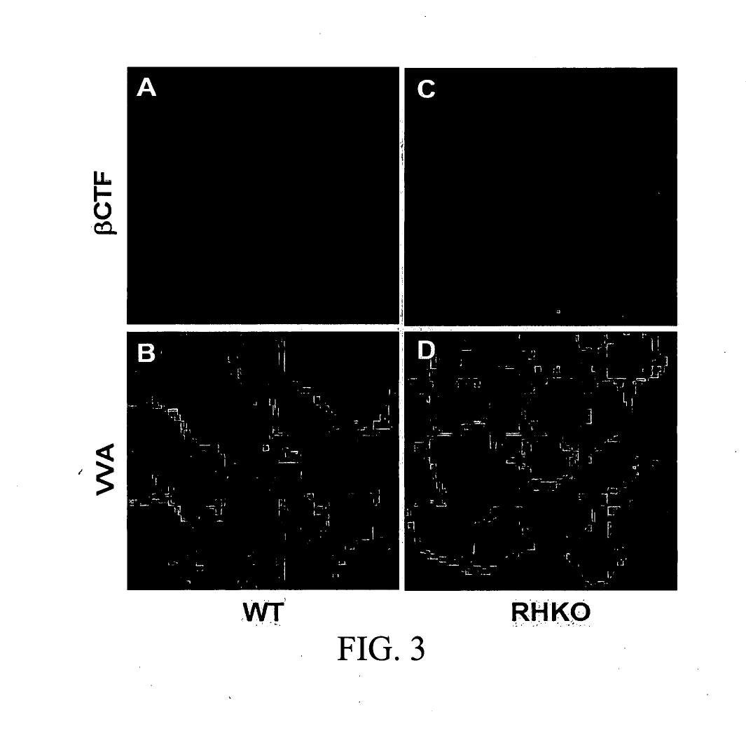

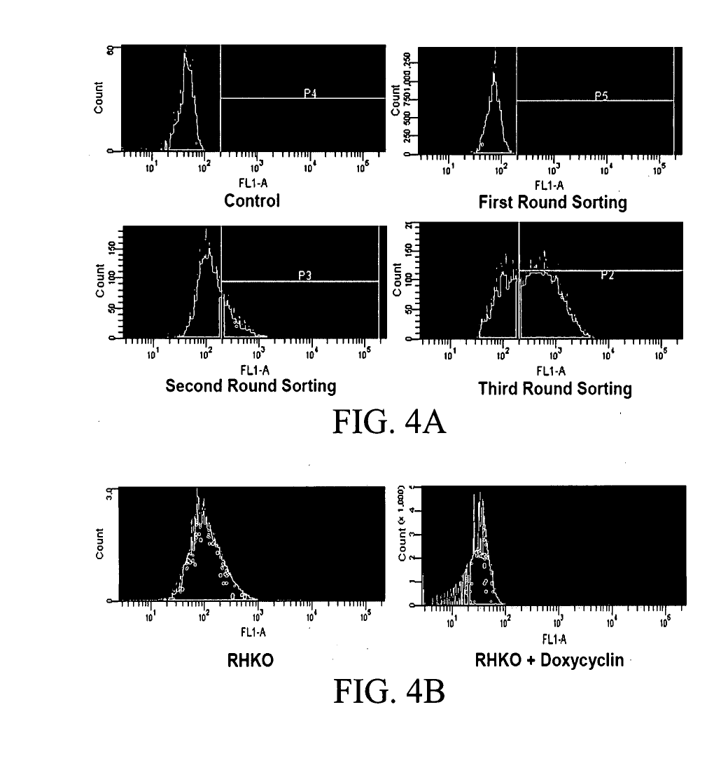

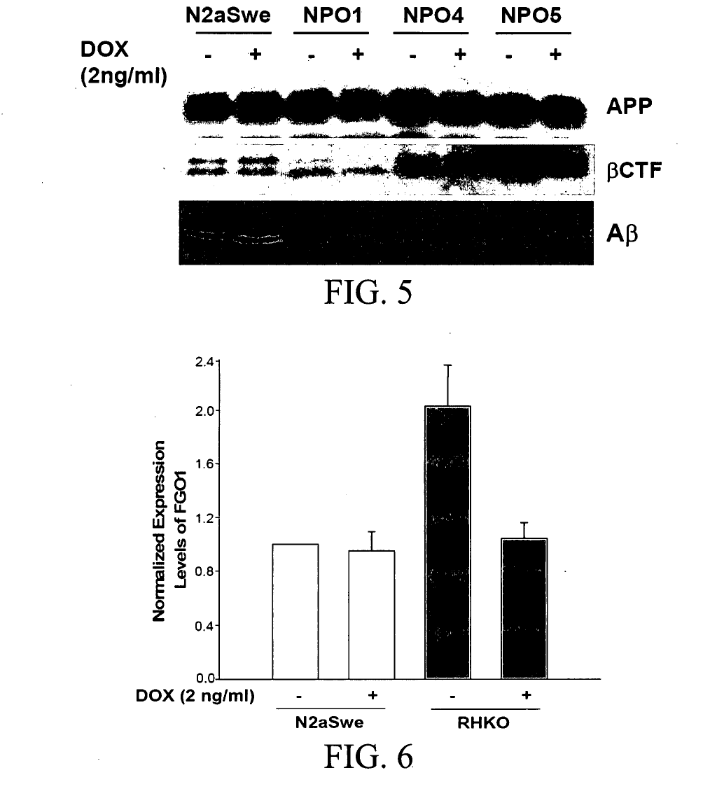

RHKO cell libraries were generated by infection of mouse neuroblastoma cells bearing a human βAPP Swedish mutant cDNA (N2aSwe) with retroviruses carrying the search vector (FIG. 2A). The search vector is composed of a tetracycline-regulated (tet-off) promoter followed by a cassette containing bacterial replication origin and antibiotic selection marker, and a hygromycin resistance gene. The tet-off promoter and the coding sequence of hygromycin resistance are in opposite direction. The transcription activity of the tet-off promoter was controlled by a chromosomal stably integrated tet specific transactivator expression vector and the addition or removal of tetracycline or doxycyclin in cell culture media. After random retroviral insertion, the stable RHKO cells were selected against hygromycin, which indicates integration of the search vector and expression of the hygromycin resistance under the control of a chromosomal promoter (FIG. 2). The RHKO screening strategy was designed to identify and isolate target genes based on their biological function. Genes were sought that involve in APP processing/Aβ generation. Of particular interest were the genes whose altered expression by the insertion of the search vector reduces Aβ generation (γ-cleavage) and hence cause accumulation of βCTF at cell surface. Therefore, the hygromycin resistant cells were live immunostained for βCTF at the cell surface with an Aβ N-terminal specific antibody (FIG. 3) and the cells were screened by multiple rounds of FACS. This phenotype of βCTF accumulation in the cell surface is also well manifested in cells deficient of PS1 (Chen, F., et al. 2000). Using this strategy it was demonstrated that cell population with βCTF accumulation can be isolated by several rounds of FACS assays, less than 0.01% of cells with βCTF accumulation after RHKO can be enriched up to 75% following three rounds of FACS assays (FIG. 4A). To minimize the variability of cell surface expression of βCTF, the tetracycline-mediated functional validation was applied in the βCTF FACS assays. Since the promoter of RHKO is tetracycline-regulated, tetracycline-mediated reversibility further validated the acumination of βCTF as a result of RHKO, and eliminates potential false-positive cells that result from cell-cell/clonal line variability of βCTF expression (FIG. 4B). Such validated RHKO cell lines were selected for further biochemical analyses, gene isolation and characterization. FIG. 5 shows an example of Western blotting analysis of three RHKO cell lines. All of them had increased Aβ and decreased βCTF levels in the presence of doxycyclin when the promoter on the search vector was turned off, indicating the observed accumulation of βCTF is a consequence of reduced Aβ generation, and caused by RHKO.

FIG. 2 shows gene screening using RHKO cell library. As shown in FIG. 2A, the host cells were infected with retroviruses carrying the search vector, the retroviruses were randomly integrated into chromosomes. The hygromycin selection marker could be expressed by the chromosomal promoter 1, and the cells can be selected by hygromycin resistance. The tetracycline-regulated promoter can transcribe antisense RNA of gene 1 and/or sense RNA of gene 2. The antisense RNA of gene 1 could knock down or shut off the expression of gene 1, and the transcription of gene 2 by the exogenous promoter could lead to overexpression of gene 2. In either scenario, the RHKO cells can be selected by designed assays for selectable phenotypes, given the genes are involved in APP processing and/or Aβ generation. As shown in FIG. 2B, work flow of screening for genes affecting Aβ generation using RHKO cells.

FIG. 3 shows accumulation of βCTF on the cell surface in RHKO cells. To visualize surface βCTF, live parental and RHKO N2aSwe cells were stained with FCA18 (Aβ1 specific antibody) at 4° C. without prior fixation or permeabilization (upper panels). Cells were also double stained with FITC-VVA (Vicia Vilosa Agglutinin, 1:1000) to stain total surface glycoproteins (lower panels).

FIG. 4 shows isolation of cell population with enhanced detection of cell surface β-CTF. As shown in FIG. 4A, RHKO library of N2a cells and control N2a cells were assayed for cell surface βCTF, positive populations of N2a cells were further enriched by second and third rounds of FACS sorting. As shown in FIG. 4B, tetracycline mediated reversibility of βCTF acumination. N2a cells isolated following 2 rounds of βCTF assays were treated without (left) and with (right) tetracycline for three days, and reanalyzed for βCTF by FACS. Y axis, number of cells; X axis, intensity of fluorescence.

FIG. 5 shows Western blotting analysis of selected RHKO cell lines. Three selected RHKO cell lines were treated with or without Doxycyclin at indicated concentration for 72 hrs before the cells were lysed, and the lysates were subjected to SDS-PAGE and Western blotting to detect full length APP and βCTF. The secreted Aβ was immunoprecipitated (then western blotted) from the media.

### 2. Example 2

### A New Gene was Cloned from an RHKO Cell Line

To isolate the genes from the screened RHKO cell lines, the purified DNA was digested and re-ligated to generate plasmids containing the search vector and chromosomal fragments of the inserted gene. Such plasmids were recovered by selecting the transformed bacteria against Chloramphenicol resistance marker. A new gene, designated as FG01, was successfully isolated from an RHKO cell line. The initial cloning and sequencing analyses revealed that the Tetracyclin-regulated exogenous promoter was inserted upstream of FG01, and likely caused overexpression of FG01. This notion was supported by real-time RT-PCR using RNAs from parental N2aSwe and the RHKO cells with or without Doxycyclin treatment. The Q-PCR results indicated that the expression level of FG01 in the RHKO cells was about 2-fold of those in parental N2aSwe and RHKO cells treated with doxycyclin, indicating that the search vector overexpressed FG01 and the overexpression can be reversed by turning off the Tetracyclin-regulated promoter using doxycyclin (FIG. 6).

FIG. 6 shows FG01 was overexpressed in the RHKO cells. Parental N2aSwe and the selected RHKO cells were treated with 2 ng/ml doxycyclin or DMSO for 72 hrs before the RNA was isolated for real-time RT-PCR to quantify the expression levels of FG01. The level of FG01 in N2aSwe cells treated with DMSO was used as control.

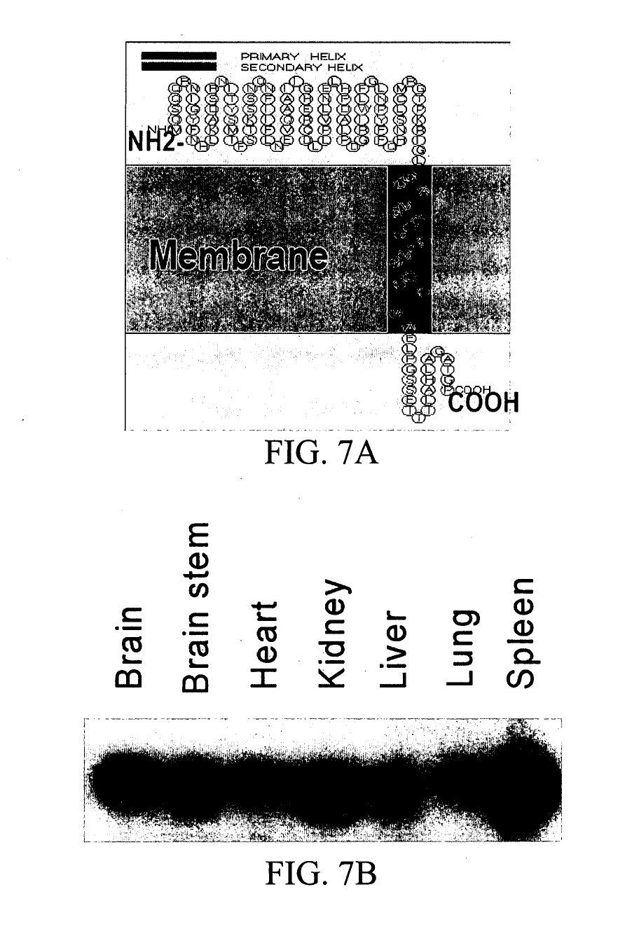

Blast search of the cloned FG01 in mouse genome found that FG01 is located on mouse chromosome 8. To confirm the identity of the gene, RT-PCR and rapid amplification of cDNA 3′ ends (3′-RACE) was performed and an approximate 400 bp fragment from N2a cell RNA cloned. The sequence of the fragment was identical to the 3′ partial sequence of a hypothetical gene cloned from mouse embryonic stem cell deposited in GenBank. Bioinformatics analyses using the postulated FG01 amino acid sequence predicted a signal peptide and a helical transmembrane domain at its N- and C-terminus, respectively (FIG. 7A, schematic drawing). A vector expressing recombinant FG01 was constructed with a His₆ tag at its C-terminus. The cellular localization of the recombinant FG01 was determined by Western blots after fractioning the transfected cells. The results showed the recombinant protein was exclusively localized in the membrane fractions. In addition, the tissue specificity of FG01 was determined by Northern blots. FG01 mRNA was expressed in all mouse tissues examined, including brain and brain stem, with the highest level in spleen (FIG. 7B).

As shown in FIG. 7A, FG01 is predicted as a transmembrane protein with a single transmembrane domain near the C-terminus. FIG. 7B shows tissue distribution of FG01. Northern blotting analysis was performed using 4 pg total RNA from the indicated mouse tissues. FG01 is expressed in the examined tissues. β

### 3. Example 3

### FG01 Inhibits Aβ Generation and Reduces GSK-3 Activities

To investigate FG01's effects on Aβ generation, the His6-tagged recombinant FG01 was overexpressed in N2aSwe cells. The levels of βCTF, APP, sAPPα and secreted and intracellular Aβ were then quantified in the transfected cells and compared to those parental cells. The results showed the levels of secreted and intracellular Aβ were reduced more than 50% in the presence of the exogenous FG01 (both Aβ40 and 42 were equally reduced), and it is correlated with the greatly accumulated βCTF and stimulated sAPPα. These results agreed to the observations in the RHKO cells whose endogenous FG01 was overexpressed by the search vector, indicating that FG01 plays a role in Aβ generation.

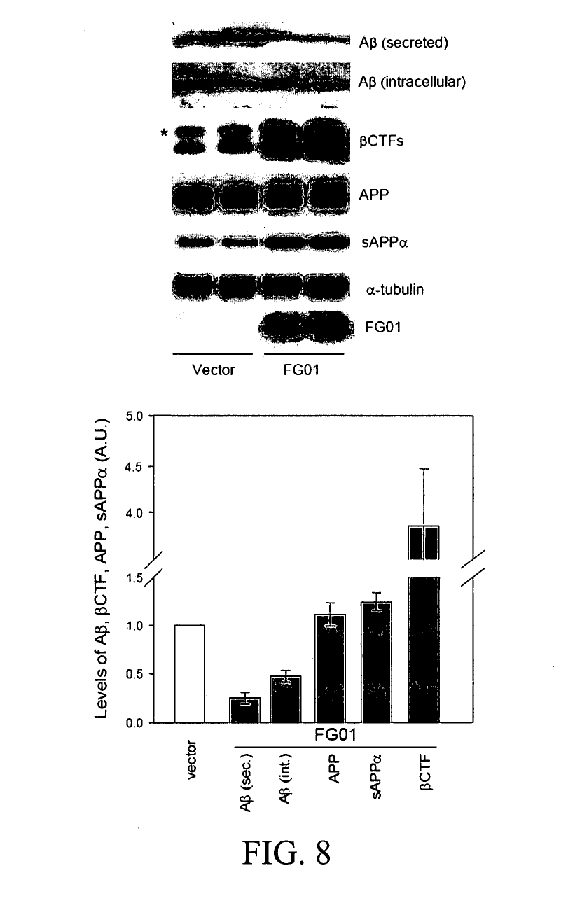

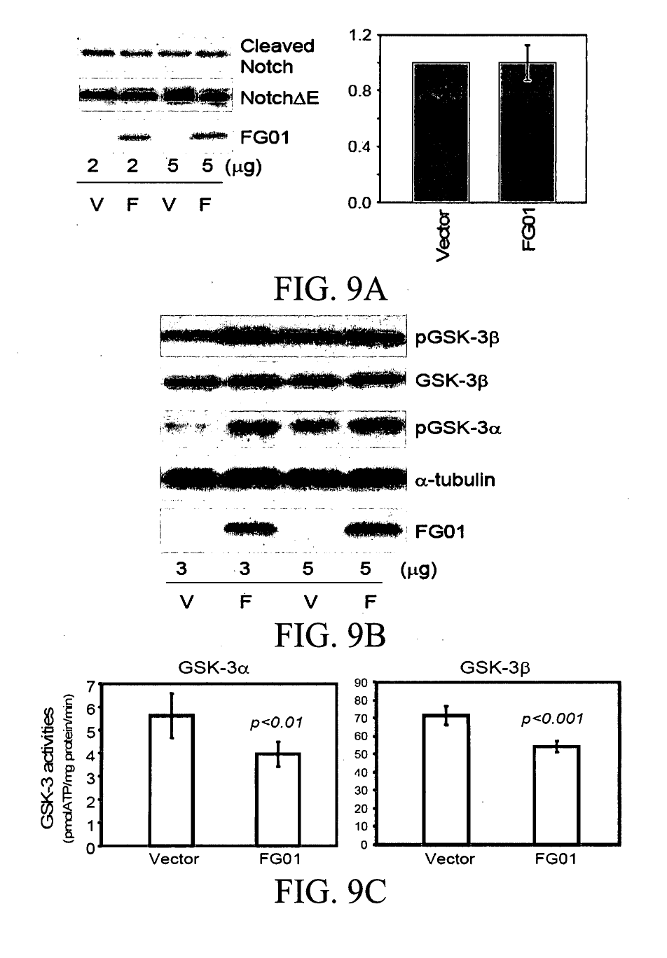

To find out whether FG01 reduces Aβ generation through affecting β-secretase activities, the BACE1 activity was examined by an in vitro assay, and it was found that FG01 overexpression did not significantly change BACE1 activity. The accumulation of βCTF can also be attributed to decrease in γ-secretase cleavage. However, the Notch cleavage was not altered by overexpression of the recombinant FG01 (FIG. 9A). Although FG01 may modulate the substrate specificity or accessibility of γ-secretase, it is equally possible that FG01 regulates other proteins/pathways that involve in APP processing and Aβ generation, such as GSK-3, which has been shown to be closely related to (reducing) Aβ generation (without affecting γ-secretase mediated Notch cleavage) and tau phosphorylation. Thus, the activities of GSK-3α/β were examined with or without overexpressing FG01. First, the levels of phospho-GSK3α at Ser21 and GSK-3β at Ser9 (the inactivated forms of GSK3) were quantified using the specific antibodies. It was found that more GSK-3α/β were phosphorylated at the sites in cells overexpressing FG01, indicating that FG01 decreased kinase activities of GSK-3. This notion was confirmed by the direct measurement of the GSK-3 kinase activities by in vitro specific kinase assay, which demonstrated about 40% decrease in GSK-3α and 30% in GSK-3β activities. These data revealed a correlation between the reduced Aβ, decreased GSK-3 activities and overexpression of FG01.

FIG. 8 shows FG01 affects Aβ generation. The His6-tagged FG01 was overexpressed in N2aSwe cells. The secreted and intracellular Aβ were immunoprecipitated by 4G8. The precipitated peptides and sAPPα, βCTF, full length APP and FG01 were analyzed by Western blots. α-tubulin was used as loading control. The levels of the proteins were quantified by densitometry and normalized and compared to those in vector transfected control.

FIG. 9 shows FG01 regulates GSK-3 but not γ-secretase activities. As shown in FIG. 9A, Notch ΔE and different amounts of FG01 or vector were cotransfected into N2aSwe cells. The levels of the recombinant Notch (NotchΔE with Myc-tag) and the γ-secretase cleaved Notch fragment (NICD) were quantified by Western blotting using myc antibody and an antibody specific to processed Notch followed by densitometry. As shown in FIG. 9B, inactivated phospho-GSK3α/β and total GSK-3β in FG01 or vector transfected N2aSwe cells were analyzed by Western blots. As shown in FIG. 9C, activities of GSK-3α/β in vector or FG01 transfected N2aSwe cells were determined by specific kinase assay (Liu, S. J., et al. 2003; Liu, S. J., et al. 2004). The results represent at least four independent experiments.

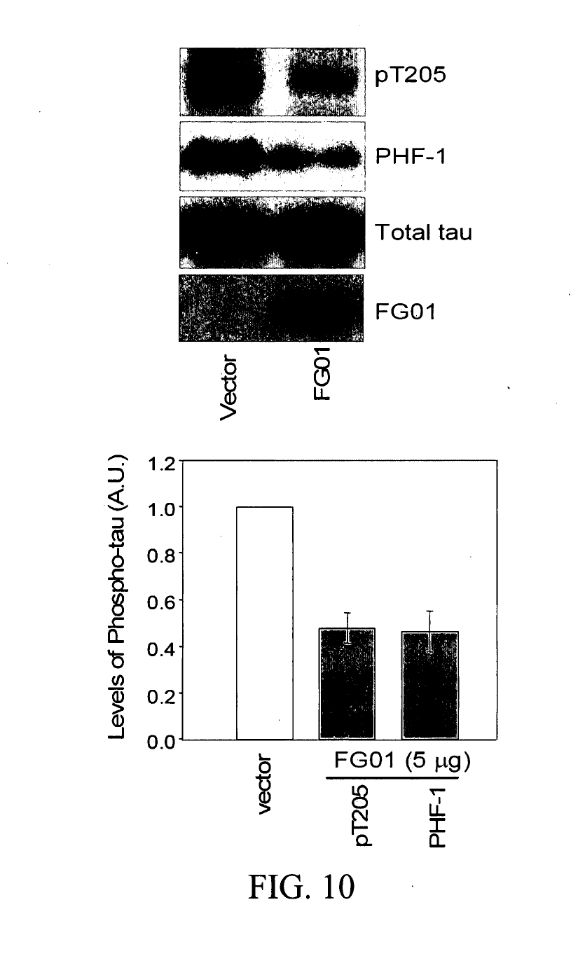

Since GSK-3 can phosphorylate tau at multiple sites, it was determined whether FG01 can affect tau phosphorylation by GSK-3. The human longest tau splicing variant T40 was cotransfected with FG01 or expression vector (control) into N2aSwe cells, and the tau phosphorylation examined at Thr205 and PHF-1 tauepitope (Ser396 and Ser404), which are GSK-3 phosphorylation targets and major paired helical filament (PHF) tau sites found in NFTs. The results indicated that overexpression of the exogenous FG01 significantly decreased tau phosphorylation at these GSK-3 targets by at almost 60%, indicating a role of FG01 in reducing tau phosphorylation along with its effect on Aβ generation (FIG. 10). In addition, little change was found in CDK5 protein level and its in vitro activity upon FG01 overexpression.

FIG. 10 shows FG01 affects tau phosphorylation. The human tau T40 was co-transfected with either FG01 or the expression vector into N2aSwe cells. The phospho-tau at Thr205 and PHF-1 and total tau proteins in the transfected cells were detected by Western blots (left). The levels of phospho-tau at the examined sites were quantified by densitometry and normalized to the total levels (right).

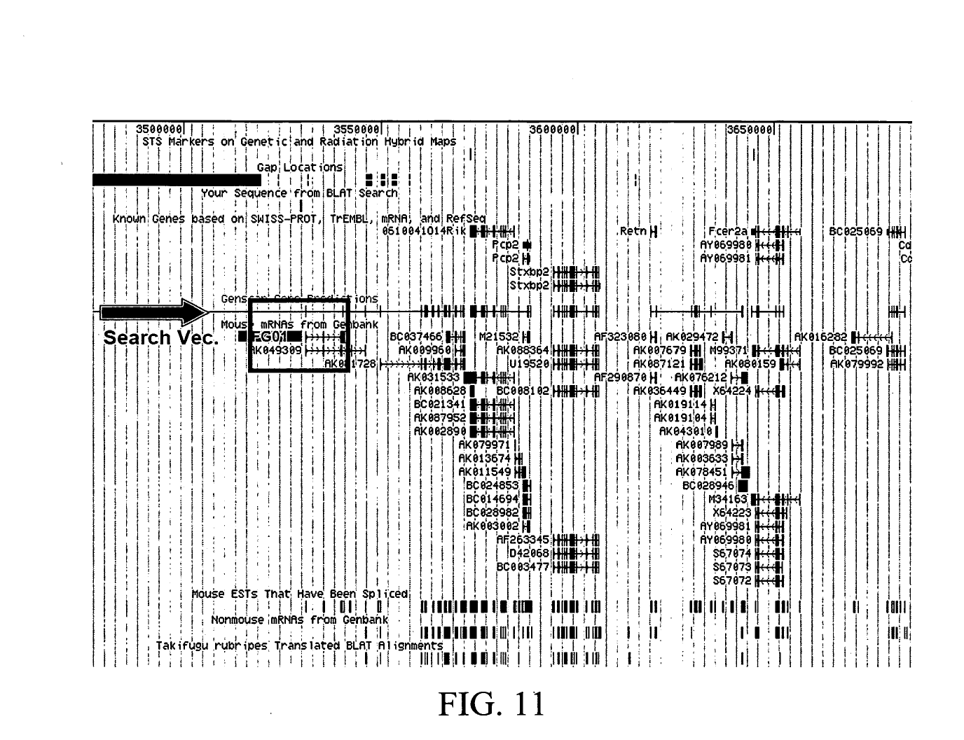

FIG. 11 shows genomic localization of FG01. FG01 was mapped at murine chromosome 8 that is boxed. The search vector (arrow) was inserted upstream of FG01 to drive overexpression of FG01.

Nucleic acid and amino acid sequences of FG01 are as follows.

FG01 nucleic acid coding sequence: (Accession No. AK049309; SEQ ID NO: 1):

ATGCAGAGCCAACAGAGAAACATTGGCTACTTTAACCACCTTAAAGCGGA
CTCCAGGAATATCACCTACAGCATGACCTTTTCGACGAAATCCAGCAACC
AGAACTTCATCATTTTCCTCAATGAAGTTCAGGCAGCCATCATTGGGCAC
GAACGCTGTGATCTTCTTCCCGTTCTTAATGAGCTGCACCCTGACGCACT
TCCTGATGGCAGAATTTGGCTGTTTGGCCTCAACCCCTACTTTTTCCAGC
ACAATTCCCTTTGCATGAGAGGCACCCCCAAACGGATTGGCCTTCAGGGC
TGTGCCCAAGTGGGCTTTCTTGTACTGTTTGTCATGCCACTTCTGATCCC
GTCGGTGACTGCGGAGCTTCCGGGCAGTTCGGAGACCACGACACTTGCCC
ATCTTGCCGGCGCCACGGGCCCCTAA

FG01 Deduced amino acid sequence (SEQ ID NO: 2):

MQSQQRNIGYFNHLKADSRNITYSMTFSTKSSNQNFIIFLNEVQAAIIGH
ERCDLLPVLNELHPDALPDGRIWLFGLNPYFFQHNSLCMRGTPKRIGLQG
CAQVGFLVLFVMPLLIPSVTAELPGSSETTTLAHLAGATGP*

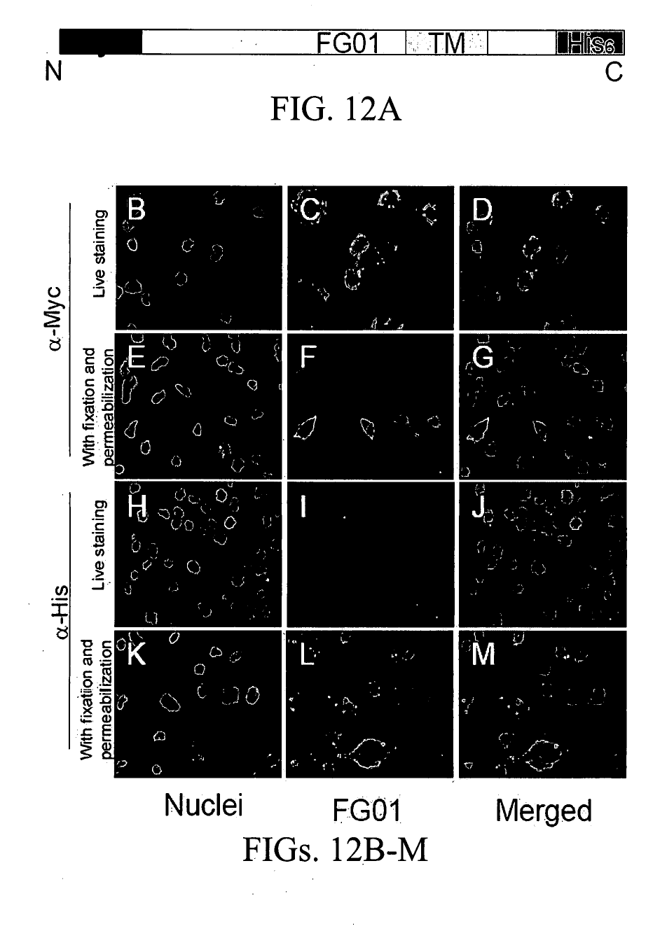

FIG. 12 shows FG01 is a type I transmembrane protein. To determine the topology of FG01, Myc and His tags were added to FG01 N- and C-terminus, respectively (FIG. 12A). After transfection of the double-tagged FG01 in N2a cells, the cells were live stained with anti-Myc (FIG. 12C) or anti-His (FIG. 12I) antibody. Another group of transfected cells were stained after fixation and permeabilization (FIG. 12F,L). Nuclei were labeled by Hoescht 33258 (FIG. 12B,E,H,K). FIGS. 12D, G, J and M are digitally merged micrographs of nuclei and FG01 staining.

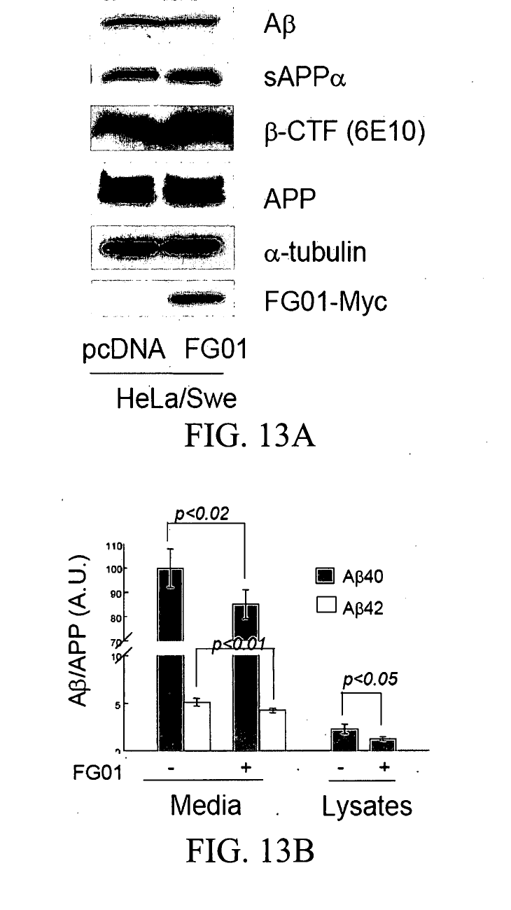

FIG. 13 shows expression of FG01 decreases the levels of Aβ and affects APP processing in human cells. As shown in FIG. 13A, Myc-tagged FG01 was expressed in HeLa cells stably expressing APP Swedish mutant. Expression of secreted Aβ, sAPPα, β-CTF, total APP, α-tubulin and FG01 were detected by Western blots. As shown in FIG. 13B, the levels of secreted and intracellular antibody species in the FG01-transfected HeLa cells were determined and compared to those in pcDNA-transfected control cells by revised ELISA. n=4.

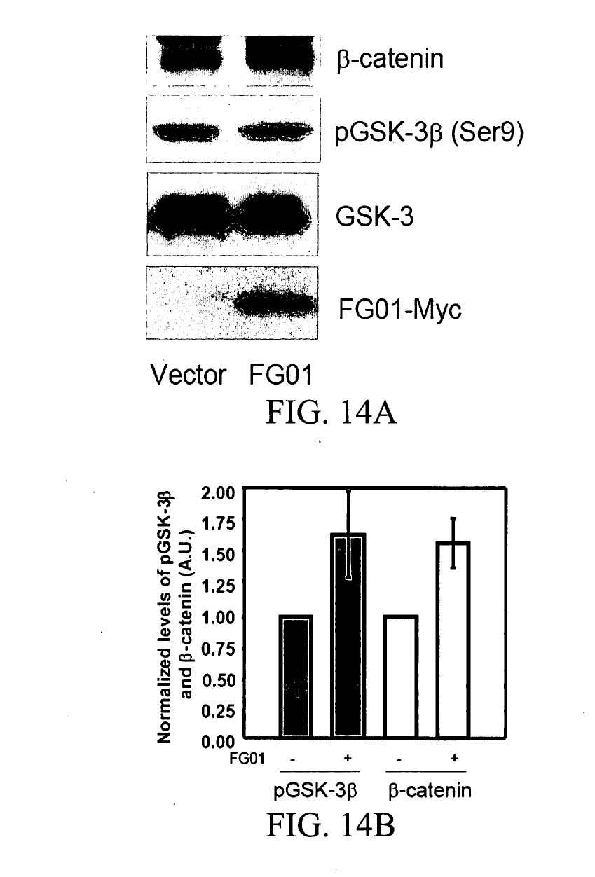

FIG. 14 shows overexpression of FG01 decreases GSK-3 activities and increases the level of β-catenin in mouse N2a cells. As shown in FIG. 14A, expression of β-catenin, inactivated phospho-GSK-3β, total GSK-3 and FG01 in transfected N2a cells were shown by Western blots. As shown in FIG. 14B, the levels of the inactivated pGSK-3β and β-catenin were quantified by densitometry. The levels of total GSK-3 were used as normalization factors.

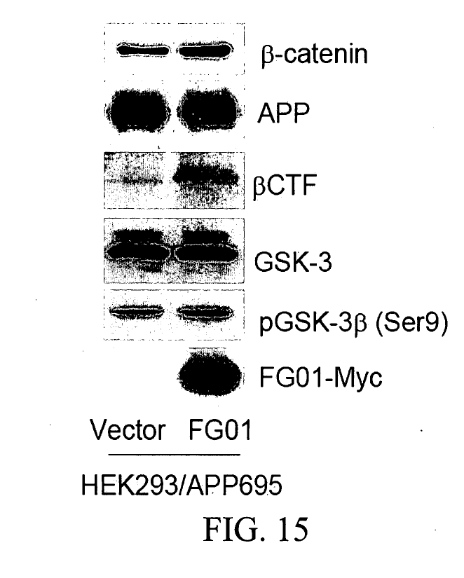

FIG. 15 shows expression of FG01 decreases GSK-3 activities and increases the level of β-catenin in human HEK293 cells. Expression of β-catenin, inactivated phospho-GSK-3β, total GSK-3, APP, β-CTF and FG01 in transfected human HEK293 cells were shown by Western blots.

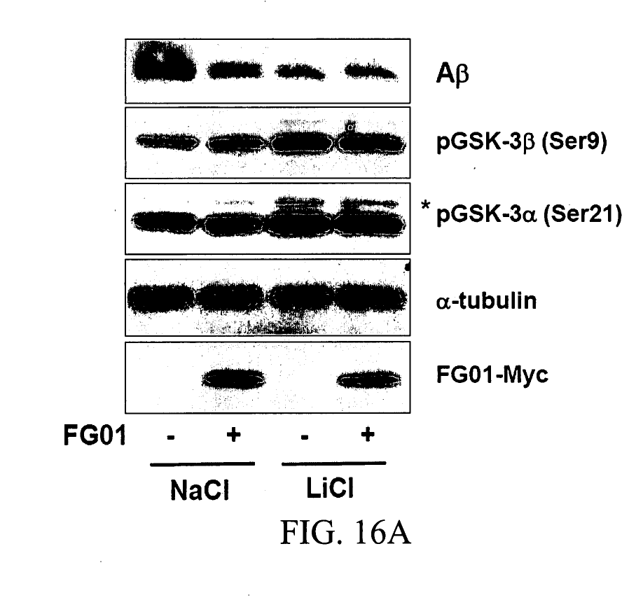

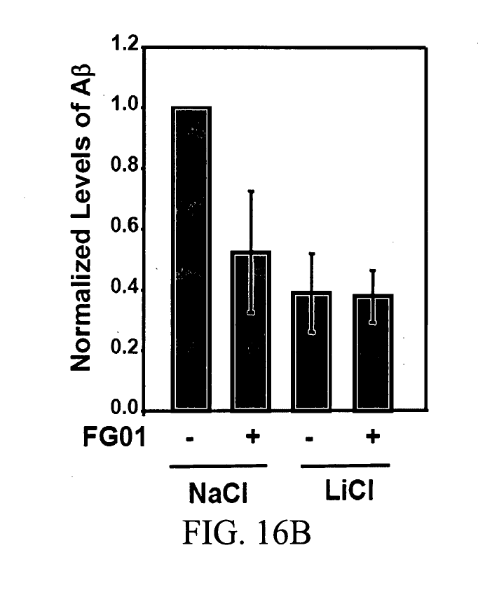

FIG. 16 shows overexpression of FG01 did not further decrease Aβ levels in LiCl-pretreated N2a cells. N2a/swe cells were pretreated with 5 mM LiCl or NaCl for 24 hrs before transfected with FG01 or pcDNA. As shown in FIG. 16A, extracellular antibody, pGSK-3β/α, α-tubulin and FG01 were detected by Western blots. As shown in FIG. 16B, levels of secreted antibody were quantified by densitometry and normalized to the amounts of α-tubulin.

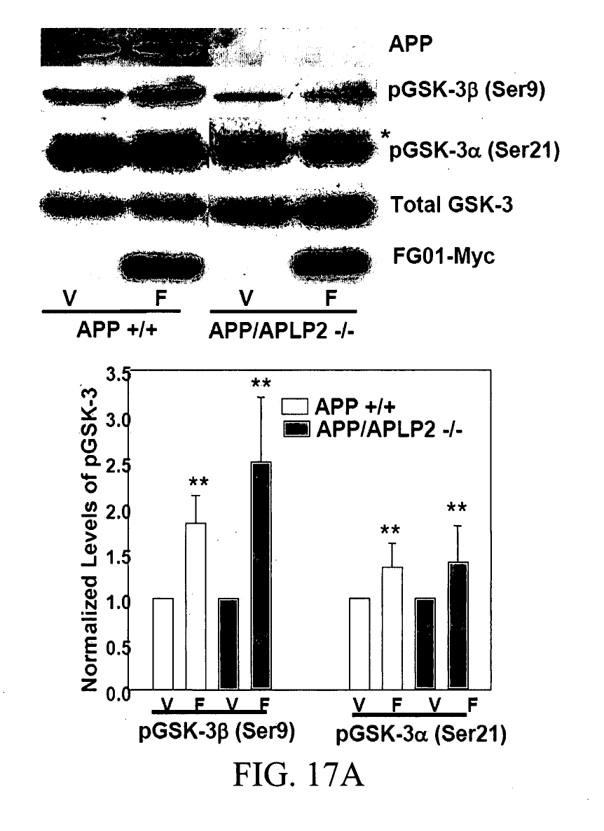

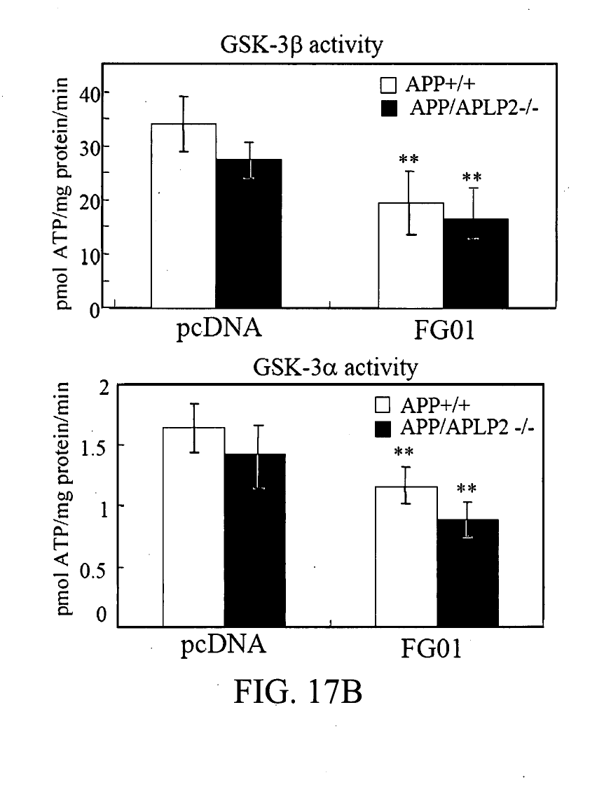

FIG. 17 shows APP/AICD is not required for FG01's regulatory effects on GSK-3 activities. As shown in FIG. 17A, FG01 or pcDNA was transfected into wild-type (+/+) or APP/APLP2 double knockout (−/−) mouse fibroblast cells. The expression of APP, phosphorylated GSK-3α/β, total GSK-3 and FG01 were shown by Western blots (left panels); and the levels of phosphorylated GSK-3 were quantified by densitometry and normalized to those of total GSK-3. As shown in FIG. 17B, activities of GSK-3α/β in vector or FG01 transfected N2aSwe cells were determined by specific kinase assay. Data represent results from three experiments, **: p<0.01.

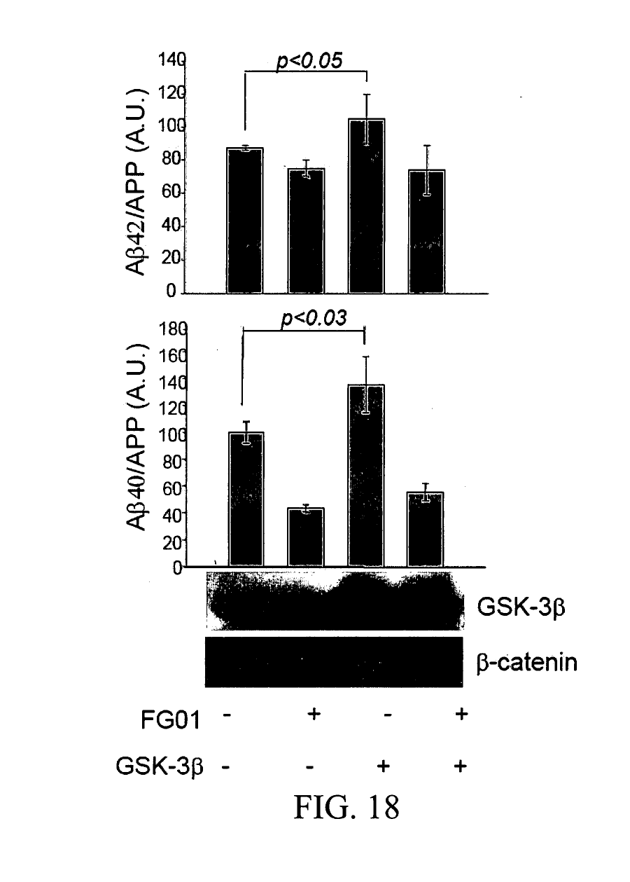

FIG. 18 shows expression of FG01 suppressed the effects of overexpression of GSK-3β on Aβ production in HeLa/swe cells. HeLa/swe cells were transfected with GSK-3β and/or FG01 as indicated. The levels of Aβ40/42 were determined by modified ELISA. The expression of GSK-3β and β-catenin were shown by Western blots. n=4.

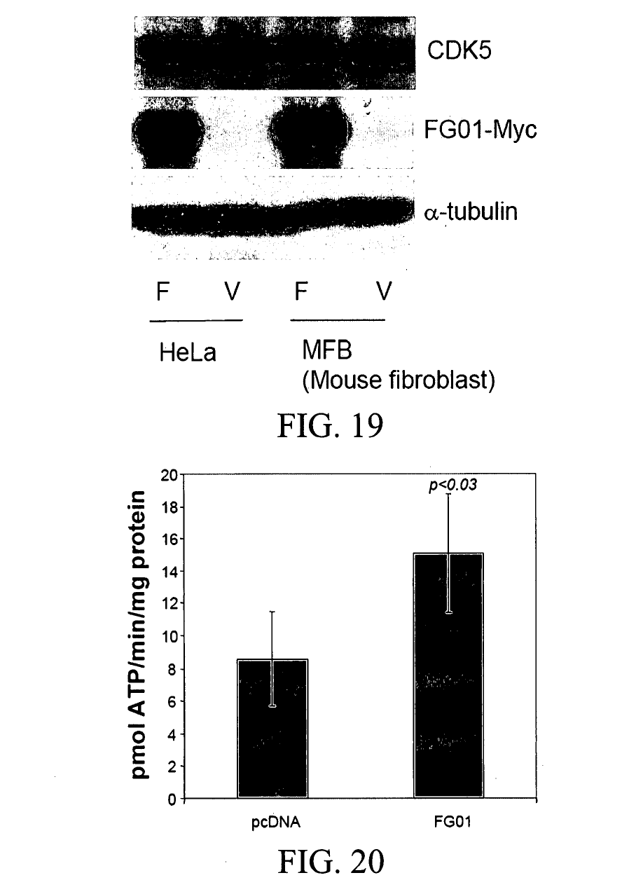

FIG. 19 shows expression of FG01 did not change the levels of CDK5 in mouse and human cells. The expression of CDK5, FG01 and α-tubulin were detected by Western blots in transfected HeLa and MFB cells.

FIG. 20 shows PKC activity was increased by overexpression of FG01. N2a cells were transfected with pcDNA or FG01. After lysis, cell lysates containing 5 mg of total proteins were incubated with synthesized PKC specific peptide substrate and ³²P-ATP. The ³²P labeled peptides were captured and the radioactivities were quantified by scintillation. The activities of PKC were represented by the normalized radioactivities of the labeled peptide substrates. n=4.

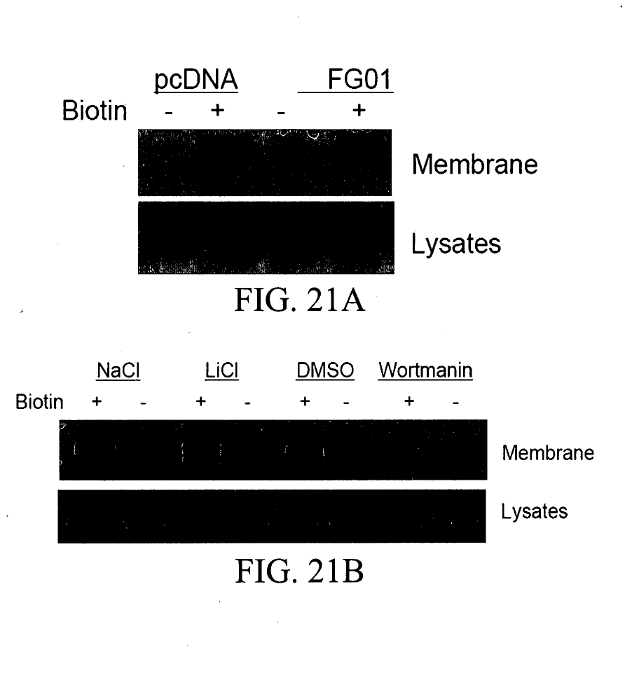

FIG. 21 shows FG01 stimulates APP trafficking to cell surface. As shown in FIG. 21A, N2a/swe cells were transfected with FG01 or pcDNA. After treated with biotin or DMSO, membrane protein and total cell lysates were subjected to Western blots for APP. As shown in FIG. 21B, N2a/swe cells were treated with NaCl, LiCl, Wortmanin or DMSO before subjected to biotinylation and subsequent Western blots for APP.

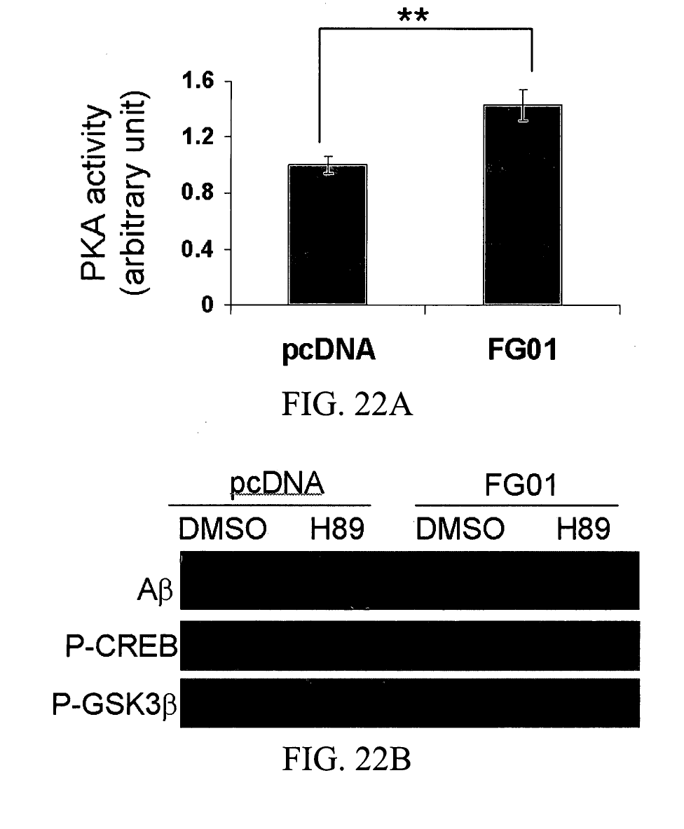

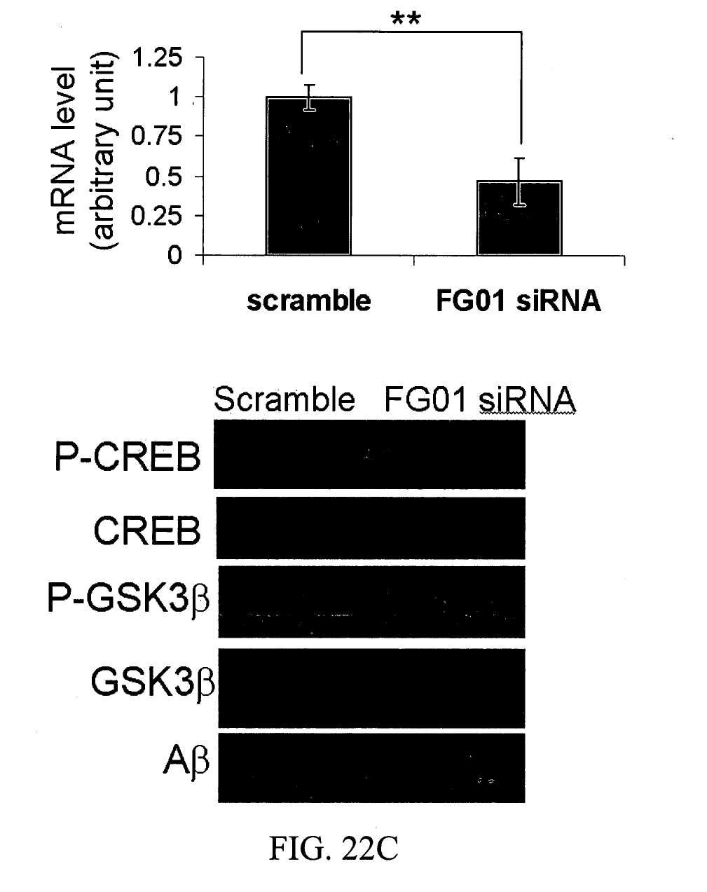

FIG. 22 shows FG01 affects Aβ generation through regulating PKA activity. As shown in FIG. 22A, N2a/Swe cells were transfected with pcDNA or FG01. After lysis, cell lysates were assayed for PKA activity, using a commercial kit and following the manufacturer's protocol (Upstate, catalog number 17-134). n=4. As shown, FG01 significantly increase PKA activity in the cells compared to control.

As shown in FIG. 22B, N2a/Swe cells were first transfected with pcDNA or FG01 and then equally split into different plates. After 48 hr incubation, cell media were changed to Opti-MEM (Invitrogen) containing DMSO or a PKA activity inhibitor H89 (2 μM) and cells were incubated for additional 4 hrs. The cell media were used for Aβ immunoprecipitation-Western blotting and cell lysates were subjected to Western blotting for p-CREB (substrate for PKA) and p-GSK3β. As shown, inhibiting PKA activity with H89 prevented serine phosphorylation of CREB and GSK3β by FG01.

As shown in FIG. 22C, N2a/Swe cells were transfected with scramble siRNA or FG01 siRNA (SEQ ID NO:20). Cells were collected for mRNA extraction, reverse-transcription, and PCR amplification with FG01 specific primers to study the knockdown efficiency (upper panel), n=3. Alternatively, cells were lysed and cell lysates subjected to Western blotting for p-CREB, CREB, p-GSK3β, and GSK3β. Aβ in conditioned media was subjected to immunoprecipitation-Western blotting (lower panels). As shown, knockdown of FG01 coincided with a concomitant decrease in GSK3β phosphorylation (inactivation). **: p<0.01. These data therefore indicate that FG01 is promoting phosphorylation (inactivation) of GSK3β through PKA. Moreover, as PKA is a cAMP-dependent protein kinase, FG01 is likely having this effect through adenylyl cyclase (AC) activation of cAMP.

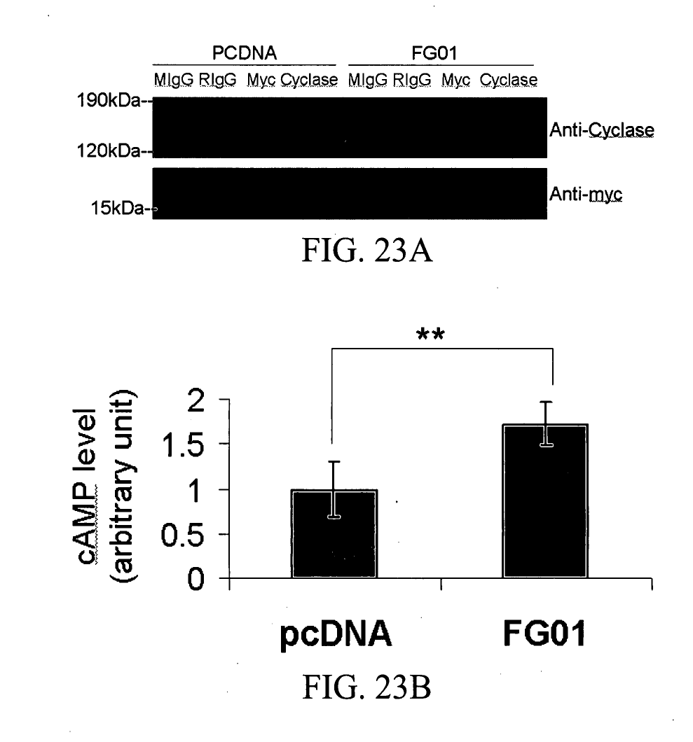

Consistently, FIG. 23 shows FG01 interacts with adenylyl cyclase (AC) and regulates the levels of cyclic AMP (cAMP). As shown in FIG. 23A, N2a cells were transfected with pcDNA or FG01. After 48-72 hrs, cells were lysed and cell lysates were subjected to immunoprecipitation with mouse IgG, rabbit IgG, myc antibody and adenylyl cyclase (AC) antibody (Santa Cruz Biotechnology, catalog number sc-1701), and then immunoblotted with antibodies against adenylyl cyclase (AC) and myc, respectively. As shown in FIG. 23B, after transfection with pcDNA or FG01, N2a cells were collected and the levels of cAMP were assayed using a commercial kit, following the manufacturer's protocol (BioVision, catalog number K371-100), n=3. **: p<0.01.

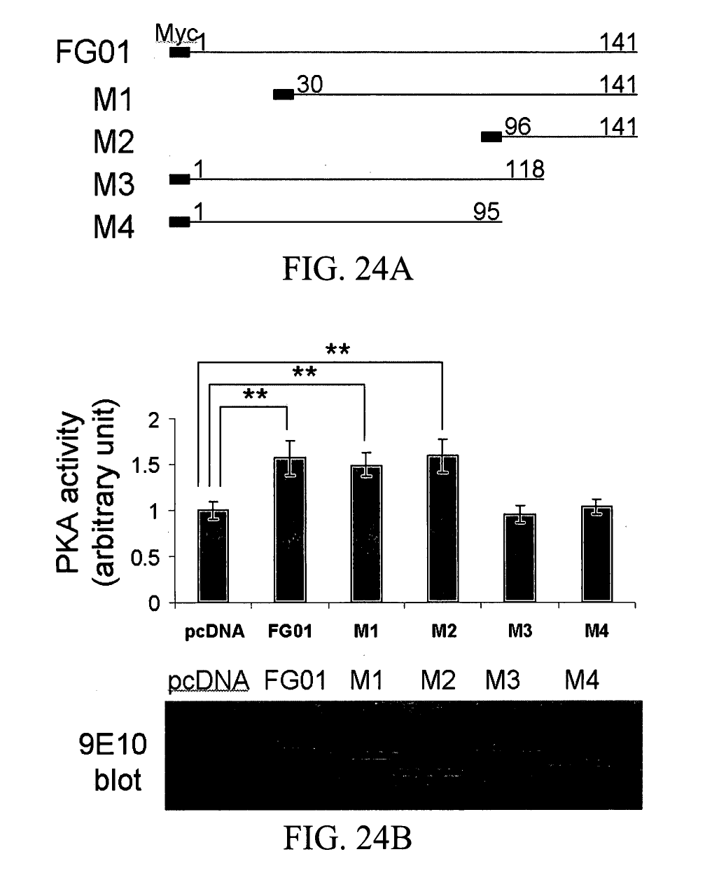

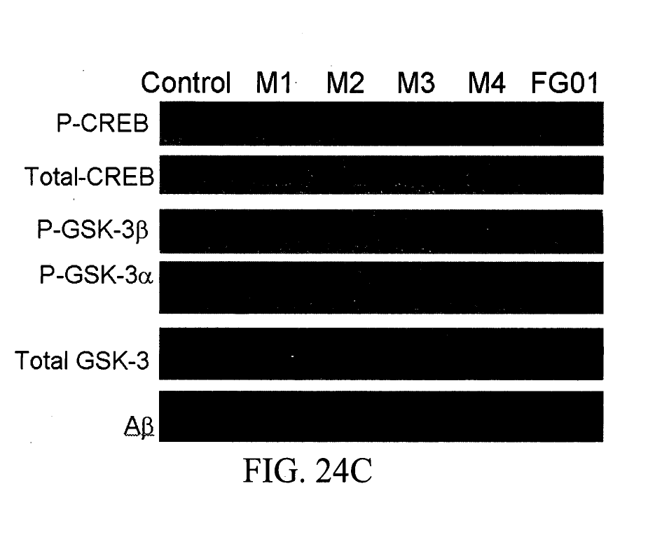

FIG. 24 shows the carboxy-terminal region of FG01 is critical for FG01's effects on the activities of PKA and GSK-3. As shown in FIG. 24A, a series of FG01 truncated fragments (M1-M4) with Myc-tagged at the amino-termini were constructed and their schemes were illustrated. As shown in FIG. 24B, pcDNA, full-length FG01 and truncated FG01 fragments were transfected into N2a/Swe cells and cell lysates were assayed for PKA activity as described in FIG. 22, n=3. **: p<0.01. Cell lysates were also subjected to Western blotting, using anti-Myc antibody 9E10 to check the expression of FG01 and its truncated forms. Moreover, cell lysates were subjected to Western blotting for p-CREB, CREB, p-GSK-3α, p-GSK-3β, and total GSK-3; and Aβ in conditioned media was subjected to immunoprecipitation-Western blotting (FIG. 24C). As shown, full-length and amin-terminal truncations induced PKA activity and GSK3β phosphorylation (inactivation).

### 4. Example 4

Retrogenes originate from mRNA-based gene duplication, evolving novel functions and genetic diversity. A limited number of retrogenes have been discovered but very little is known about their functions, especially those involved in disease pathogenesis. This example describes the identification of, analysis of, and demonstration of treatment using a novel gene fg01 in mice that originated through retroposition of ribosomal protein S23 mRNA. Remarkably, fg01 is reversely transcribed, relative to its parental gene, yielding a structurally unrelated, yet functional protein. It was demonstrate that FG01 ameliorates Alzheimer's disease (AD)-like pathologies, β-amyloid (Aβ) generation and tau phosphorylation, by interacting with adenylate cyclases to activate cAMP/PKA. This leads to inhibition of GSK-3 activity, which modulates Aβ generation and tau phosphorylation. The function of fg01 is demonstrated in cells of various species including human, and in transgenic mice expressing brain-specific fg01. Furthermore, the AD-like pathologies of the triple transgenic AD mice were improved after crossing them with the fg01 transgenic mice. This example uncovers a novel retrogene and its role in regulating protein kinase pathways and disease pathogenesis.

i. Materials and Methods

a. Cells, Antibodies, and Reagents

Maintenance of mouse neuroblastoma N2a cells, N2a cells stably expressing human APP Swedish mutation (N2aSwe), human HeLa cells stably expressing human APP Swedish mutation (HeLaSwe), and rat PC12 cells has been described (Lin et al., 2007; Wang et al., 2006; York et al., 2000). Phoenix-Ampho helper cells were grown in Dulbecco's modified Eagle's medium supplemented with 10% fetal bovine serum. Antibodies used were: anti-Myc (9E10), anti-adenylate cyclases and anti-His from Santa Cruz Biotechnology; anti-GSK-3α, anti-GSK-3β, anti-phospho-GSK-3α/β (Ser21/9), anti-CREB, anti-phospho-CREB (Ser133), anti-PSD-95, and anti-synapsin from Cell Signaling Technology; anti-Aβ (6E10) from Covance; anti-pT205 tau and anti-total tau from Abcam; anti-tau-1 from Chemicon; anti-PHF-1 tau from P. Davies at Albert Einstein School of Medicine, anti-α-tubulin from Sigma, and the FCA18 antibody specifically recognizing the N-terminus of APP βCTF from F. Checker at Institut de Pharmacologie Molecularie et Cellulaire du CNRS (Ancolio et al., 1999). The rabbit polyclonal antibody 369 against the APP C-terminus (Xu et al., 1997) and the anti-FG01 antibody were developed in our laboratory. PKA inhibitor H89 and GSK-3 inhibitor lithium chloride were from Sigma.

b. Random Homozygous Knockout (RHKO) Strategy for Screening Aβ-Reducing Genes

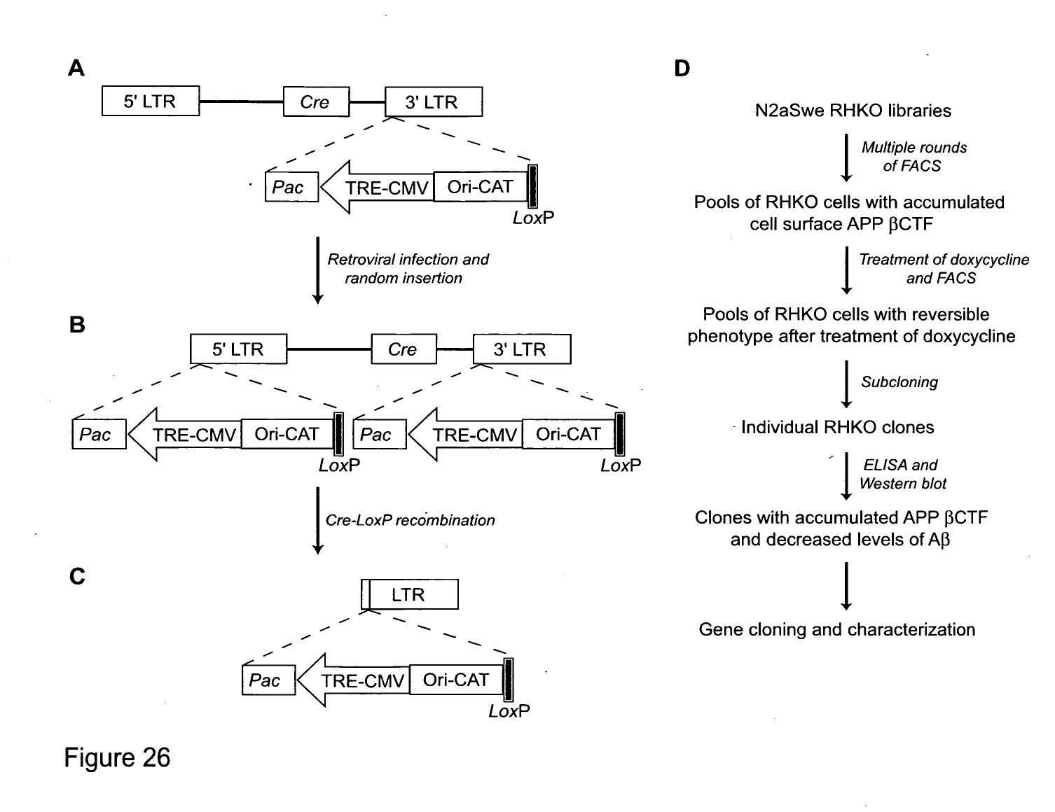

A new RHKO gene search vector was constructed from the original pLLGSV vector (Li and Cohen, 1996). In both the 5′LTR and the 3′LTR regions of the new RHKO gene search vector, there is a sequence containing a puromycin N-acetyl-tranferase gene (pac), a TRE (tetracycline-regulated element, tet-off) regulated CMV promoter driving the pac gene, a plasmid replication origin and a chloramphenicol resistance marker (Ori-CAT), and a LoxP site. In addition, there is a Cre recombinase gene (Cre) between the 5′LTR and the 3′LTR (FIG. 26A). This new RHKO gene search vector was transfected into Phoenix-Ampho help cells. Generated infectious retrovirus in the cell culture supernatant were harvested and used to infect N2aSwe cells.

The infected N2aSwe cells with RHKO vector integration were selected with puromycin, live-stained with fluorescence-labeled APP βCTF antibody FCA18 (Ancolio et al., 1999), and subjected to multiple rounds of FACS sorting for cells with accumulated cell surface APP βCTF. The positive sorted cells were then treated with doxycycline (a derivative of tetracycline) and sorted for cells whose surface APP βCTF level was reversed back to background level in the presence of doxycycline. Resultant cells were cloned individually and assayed by ELISA and Western blotting to confirm surface accumulation of APP βCTF and reduction of Aβ generation. Positive candidate cell clones were further characterized and used for gene isolation.

c. FG01 Gene Cloning

Genomic DNA was extracted from FG01 cells, digested with restriction enzyme BamHI or HindIII, and self-ligated overnight with T4 ligase. The ligated DNA was precipitated, dissolved in TE buffer and electroporated into DH10B ElectroMax competent cells. The plasmid DNA from individual colonies was prepared for DNA sequencing. The target gene was identified by using UCSC Genome Browser Program.

d. Sequence Analysis

GenBank database was blasted with fg01 cDNA sequence to explore its origination. Homologous sequences between fg01 and mouse RPS23 were aligned manually. Sequence similarity between fg01 and RPS23 cDNA sequences of humans, mice and rats were compared using their homologous regions. Human RPS23 cDNA sequence was used to screen for RSP23 retroposition sites in the human genome. For identified RPS23-like sequences in the human genome, we selected the fragment containing 100 Kb each of the 5′ and the 3′ region adjacent to these sequences for gene prediction, using Genscan (Burge and Karlin, 1997; Burge and Karlin, 1998). Potential transmembrane region in the FG01 protein was predicted using PredictProtein (Rost et al., 2004).

e. Membrane Fractionation

N2a cells were transfected with FG01, APP or SMAD3 expression vectors (all Myc-tagged). After 48 hrs, cells were washed with ice-cold phosphate-buffered saline, collected with homogenization buffer (10 mM Tris-HCl, pH 7.4, 1 mM EDTA, 200 mM sucrose, 1 mM phenylmethylsulfonyl fluoride) and homogenized with a ball bearing cell cracker. Samples were centrifuged at 900×g for 10 min to remove cell debris and nuclei. Supernatants were centrifuged at 100,000×g for 60 min at 4° C. After transferring the supernatant (cytosol) to a new tube, the pellet was washed and re-suspended with an equal volume (to that of cytosol) of homogenization buffer.

f. Biotinylation

FG01 transfected N2a cells were washed with ice-cold phosphate-buffered saline containing 1 mM each of CaCl₂ and MgCl₂ and incubated at 4° C. with 0.5 mg/ml Sulfo-NHS-LC-biotin (Pierce) for 20 min and the process repeated once. Cell lysates were prepared in Nonidet P-40 lysis buffer. After affinity precipitation with streptavidin beads (Pierce), biotinylated proteins were eluted with SDS-PAGE sample buffer (Invitrogen) and loaded directly on SDS-PAGE gels for electrophoresis followed by Western blot analysis with the Myc antibody.

g. Immunofluorescence Microscopy

For cell surface immunostaining of FG01, N2a cells were first transfected with the Myc-FG01-His₆ plasmid. Cells were then directly incubated with Myc or His6 antibody at 4° C. for 2 hrs, followed by washing, fixation, and permeabilization. In some experiments, cells were permeabilized before incubating with antibodies. Treated cells were incubated with Alexa Fluor 488-conjugated secondary antibody and DAPI. Specimens were examined and fluorescence images collected using a Zeiss fluorescence microscope with AxioVision software.

h. Aβ Elisa Assay

HeLaSwe cells were transfected with FG01 or controls. Conditioned media and lysates from these cells were collected. The levels of Aβ40 and Aβ42 were quantified using ELISA kits (Invitrogen), following the manufacturer's protocols.

i. Pharmacological Treatments

N2aSwe cells were transfected with control vector or FG01 and then equally split. Four hours before collection, cells were treated with the GSK-3 inhibitor lithium chloride (5 mM) or sodium chloride (5 mM, as control). Alternatively, cells were treated with a PKA inhibitor H89 at 10 μM or with DMSO for control.

j. FG01 RNA Interference and Quantitative Real-Time PCR

The mouse FG01 siRNA used was: 5′-UACUGUUUGUCAUGCCACUUCUGAU-3′ (SEQ ID NO:20). The control siRNA was from Invitrogen. siRNA was transfected into N2a cells using Lipofectamine RNAiMAX reagent (Invitrogen), following the manufacturer's protocol. After FG01 RNA interference, total RNA was extracted from N2a cells by Trizol reagent (Invitrogen). After reverse transcription into first strand cDNA using standard conditions, samples were analyzed independently by real-time PCR using an iCycler iQ with SYBR green supermix (Bio-Rad). The FG01 primer pair used for amplification was: 5′: TGTTGCATACACATACATGC (SEQ ID NO:21) and 3′: TCATTAAGAACGGGAAGAAG (SEQ ID NO:22). A pair of β-actin primers served as controls (Zhang et al., 2007).

k. In Situ Hybridization

Histological sections from 2-month-old C57B16 mice were used for in situ hybridization reactions. Digoxygenin-labelled sense and antisense probes were generated for FG01 (corresponding to nucleotides 1-641 of NM_{—}001024728), and the hybridization signal was detected using an alkaline-phosphatase-conjugated anti-digoxygenin antibody and BCIP/NTB (Roche).

l. Crossing Brain-Specific FG01 Transgenic Mice with 3×TG AD Mice

Brain-specific FG01 transgenic mice were generated using a FG01 expression cassette. FG01 has an N-terminal Myc tag and is driven by the human Thy-1 promoter for neuron-specific expression. A SV40 polyA fragment is linked at the 3′ end for enhancing expression. Genotyping P5 primer was complementary to hThy1 sequence. Genotyping P3 primer was complementary to SV40/Poly A sequence. Reverse transcription primer RT-P5 was complementary to sequence on the 5′ side of the FG01 sequence. Hemizygous FG01 transgenic mice were crossed with homozygous triple transgenic (3×Tg) AD mice harboring mutations in human APP and tau genes on a presenilin 1 (PS1) mutant background (Oddo et al., 2003). Procedures involving animals and their care conformed to institutional guidelines (Animal Resources Department at Burnham Institute for Medical Research).

m. Immunohistochemistry

FG01/3×Tg mice and littermate controls on a 3×Tg background were sacrificed at 11 months of age. Half of the brain was used for immunoblot analysis and the other half was paraffin-embedded for immunohistochemistry. Sagittal brain sections (4 μm) were deparaffinized, hydrated, and then immunostained with anti-Aβ antibody (6E10), anti-phosphorylated tau antibody (PHF-1), or anti-synapsin antibody. After additional incubation with biotinylated secondary antibody, samples were incubated in ABC Elite (HRP) reagent (Vector Laboratories). Reactions were visualized by developing in DAB substrates (Vector Laboratories). All samples were visualized under a light microscope.

n. In Vitro Activity Assays and cAMP Assay

Commercial kits were used to assay in vitro activities of GSK-3β (Sigma) and PKA (Upstate). For GSK-3α activity, a commercial GSK-3β activity assay kit was used but the procedure to immunoprecititate GSK-3β was replaced with immunoprecipitation of GSK-3α using an anti-GSK-3α antibody (Cell Signaling). cAMP levels were assayed using a commercial kit (Biovision).

o. Co-Immunoprecipitation

Cells transfected with FG01 were lysed in either CHAPSO buffer (1% CHAPSO, 25 mM HEPES, pH7.4, 150 mM NaCl, and 2 mM EDTA supplemented with protease inhibitors) or in NP40 buffer (1% NP40 in phosphate buffered saline, supplemented with protease inhibitors). Lysates were immunoprecipitated using mouse IgG, rabbit IgG, and antibodies against Myc or adenylate cyclases and Trueblot™ IP beads (eBioscience), followed by Western blot with antibodies against Myc or adenylate cyclases.

p. FG01 Antibody Development

A synthetic peptide (NIGYFNHLKADSRN) derived from sequences near the FG01 N-terminus was used to immunize rabbits. Sera were IgG-purified followed by peptide purification.

q. β-Secretase Activity Assay

The in vitro activity of β-secretase (BACE1) was assayed using a commercial kit (Calbiochem), following the manufacturer's protocol.

r. γ-Secretase Cleavage on Notch.

N2a cells were transfected with Myc-tagged Notch1 NΔE construct lacking the ectodomain, which allows ligand-independent processing by γ-secretase to produce NICD (Schroeter et al., 1998). NICD generation was analyzed by immunoblot with an antibody specifically recognizing the cleaved Notch product NICD (Cell Signaling Technology).

s. CDK5 Activity

The in vitro activity of CDK5 was assayed as described (Han et al., 2005). Briefly, CDK5 protein was immunoprecipitated and incubated with histone-H1 in [γ-³²P]ATP-containing buffer. After reaction termination, samples were subjected to SDS-PAGE and autoradiography. Results were quantified using a PhosphoImager (Molecular Dynamics).

t. Construction, Genotyping, and Identification of Brain-Specific FG01 Transgenic Mice

N-terminally Myc-tagged FG01 cDNA was cloned into pHZ04 construct between the human Thy-1 promoter and the SV40 polyA regions. Purified, linearized minigenes containing these sequences were microinjected into prenuclear embryos from superovulated C57B16 females. Two primers (Genotyping-P5: 5′-GAGGCCCGAATTCTCGCCGCCACC-3′; SEQ ID NO:25), and Genotyping-P3: 5′-GGATGCGCGGATAGCCGCTGCTGG-3′; SEQ ID NO:26) located within the human Thy-1 promoter and the SV40 regions, respectively, were used for genotyping. Brain tissues from confirmed transgenic mice and littermate controls were used for RNA extraction, reverse transcription and PCR with RT-P5 primer (5′-AGAATTTGGCTGTTTGG-3′; SEQ ID NO:27) located within the FG01 cDNA region paired with the Genotyping-P3 primer to verify Myc-FG01 expression. Alternatively, brain lysates were immunoprecipitated and analyzed by Western blot with the Myc antibody to verify Myc-FG01 expression. Procedures involving animals and their care conformed to institutional guidelines (Animal Resources Department at Burnham Institute for Medical Research).

ii. Results

a. Genome-Wide Screening for Genes that Regulates Aβ Generation

It has been shown that reduction of Aβ levels is accompanied by cell surface accumulation of APP βCTF (the product of β-cleavage and immediate substrate for γ-cleavage), which is readily detectable in cells deficient in PS1 (Chen et al., 2000); and these cells can be identified using an antibody specifically recognizing the N-terminus of APP βCTF (FCA18) (Ancolio et al., 1999). Based on this observation, RHKO was adapted as a high throughput screen to search for genes that regulate Aβ generation.

The original vector pLLGSV (Li and Cohen, 1996) was modified to increase efficiency of retroviral integration and gene recovery. The new RHKO gene search vector contains modified LTRs and utilizes the Cre-LoxP mediated recombination to minimize promoter interference in provirus and to facilitate genomic DNA cloning (FIG. 26A). This vector was transfected into Phoenix-Ampho cells for viral packaging. Harvested retrovirus was used to infect mouse neuroblastoma N2a cells stably expressing the human APP Swedish mutation (N2aSwe). After random insertion, the provirus (FIG. 26B) expressed Cre recombinase for recognition and recombination of the two LoxP sites located in the 5′LTR and 3′LTR, respectively, generating the final integrated provirus (FIG. 26C). A tetracycline regulated promoter (TRE-CMV) promoter in the final integrated provirus drives the expression of pac for puromycin selection and initiates transcription into flanking chromosomal gene that can either overexpress, when TRE-CMV is in the same orientation, or suppress (by expressing antisense transcripts) when TRE-CMV is in the opposite orientation relative to the flanking gene. Moreover, transcription of the tetracycline-regulated (tet-off) transactivator can be reversed in the presence of tetracycline (or doxycycline).

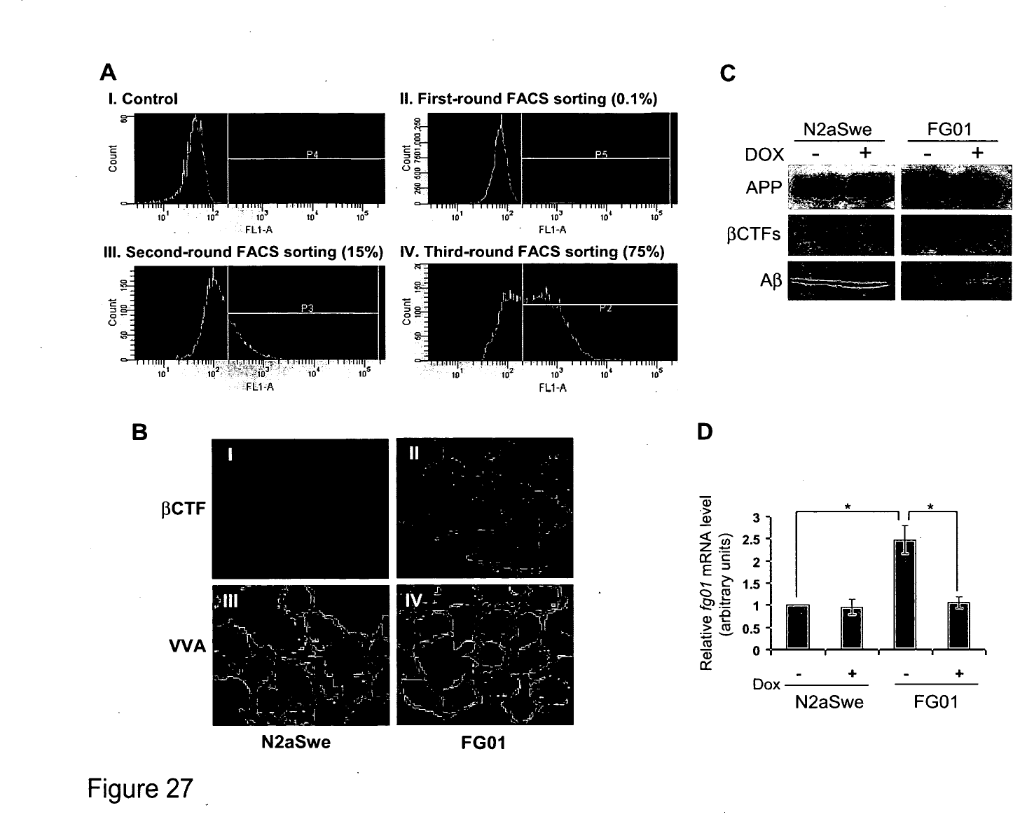

Therefore, N2aSwe cells with stable integration of the RHKO search vector were first acquired by puromycin selection. These cells were live-immunostained with fluorescently labeled FCA18 antibody followed by multiple rounds of FACS sorting to enrich for cells showing surface APP βCTF accumulation. Less than 0.01% of cells showing βCTF accumulation after first round of FACS sorting were enriched up to 75% following another two rounds of FACS sorting (FIG. 27A). These cells were then sorted with additional FACS in the presence of doxycycline for a reversion of cell surface APP βCTF to background level to eliminate potential false positive. The final sorted cells were cloned individually, propagated and assayed for both accumulated cell surface APP βCTF and reduced Aβ generation. This screening strategy is shown in FIG. 26D.

b. Identification of the FG01 Gene

One clone, FG01, was isolated for the high level of cell surface APP βCTF and the significant reduction of Aβ generation (FIGS. 27B and 27C). Both phenotypes were reversed by doxycycline treatment, validating that the effects were indeed a result of RHKO rather than cell-cell/clonal variation in APP/βCTF expression or any random mutagenesis (FIG. 27C). Subcloning and sequence analyses revealed that the RHKO vector was inserted into chromosome 8 at a site ˜1.2 Kb upstream of the C330021F23Rik gene (GenBank ID: 546049), which has no known function. This gene is designated herein as fg01. The upstream location and the same orientation of the inserted RHKO vector strongly indicated that fg01 was likely overexpressed in this cell clone. This notion was supported by real-time reverse transcription-PCR (RT-PCR) using RNAs from parental N2aSwe and the FG01 RHKO cell clone, which showed that fg01 was overexpressed in the FG01 RHKO cell clone and its overexpression was reversed by doxycycline treatment (FIG. 27D).

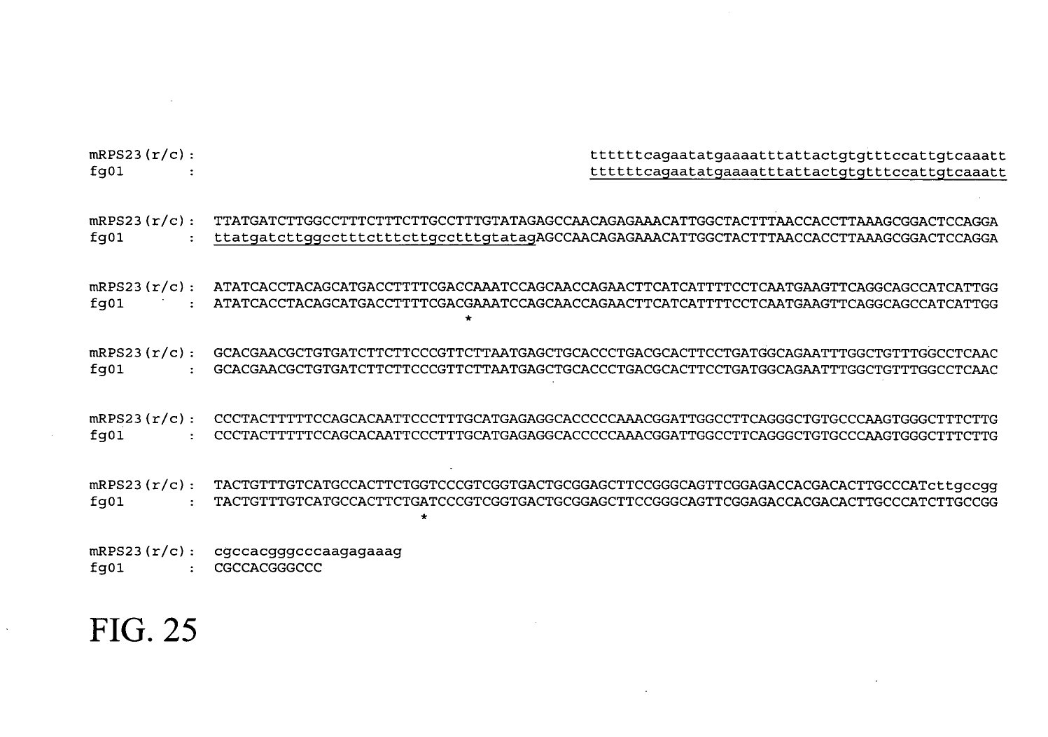

The fg01 gene is predicted to encode a 141 amino acid-long hypothetical protein that is designated herein as FG01. Interestingly, analyses of multiple genome databases (GenBank, UCSC Genome Browser and Ensemble Genome Browser) with the FG01 protein sequence identified no FG01 homologs in other species including humans and rats. Further analysis with the fg01 gene sequence showed that the predominant protein-encoding region of the fg01 gene was highly homologous to the reverse and complementary sequence of the mouse ribosomal protein S23 (RPS23) mRNA (FIG. 25). The phylogenetic relationships among fg01 and human, rat and mouse RPS23 genes (hRPS23, rRPS23, and mRPS23) based on their sequence identity to mRPS23 within the homologous region are mRPS23 to fg01, 99%; mRPS23 to rRPS23, 94%; mRPS23 to hRPS23, 88%. Thus, the similarity between fg01 and mouse RPS23 was even higher than those between mouse RPS23 and rat or human RPS23, indicating that fg01 originated from mouse RPS23 after the divergence of mice and rats. New genes can originate through different mechanisms (Long et al., 2003). However, the presence of the mouse RPS23 untranslated regions (UTRs) and the absence of the mouse RPS23 introns in the homologous regions between fg01 and mouse RPS23 indicates that fg01 originated through retroposition of the mouse RPS23 mRNA, which recruited regulatory units and additional protein-encoding sequence near the retroposition site. But transcription of fg01 is reversed compared to RPS23. To search for human homologs of fg01, the human genome was scanned with the human RPS23 cDNA sequence and identified several RPS23 retroposition sites. However, computational gene prediction of these sites revealed no functional fg01-like genes. RT-PCR was carried out with primers binding regions right next to these human RPS23 retroposition sites and positive amplification was not obtained.

c. FG01 is a Type III Transmembrane Protein

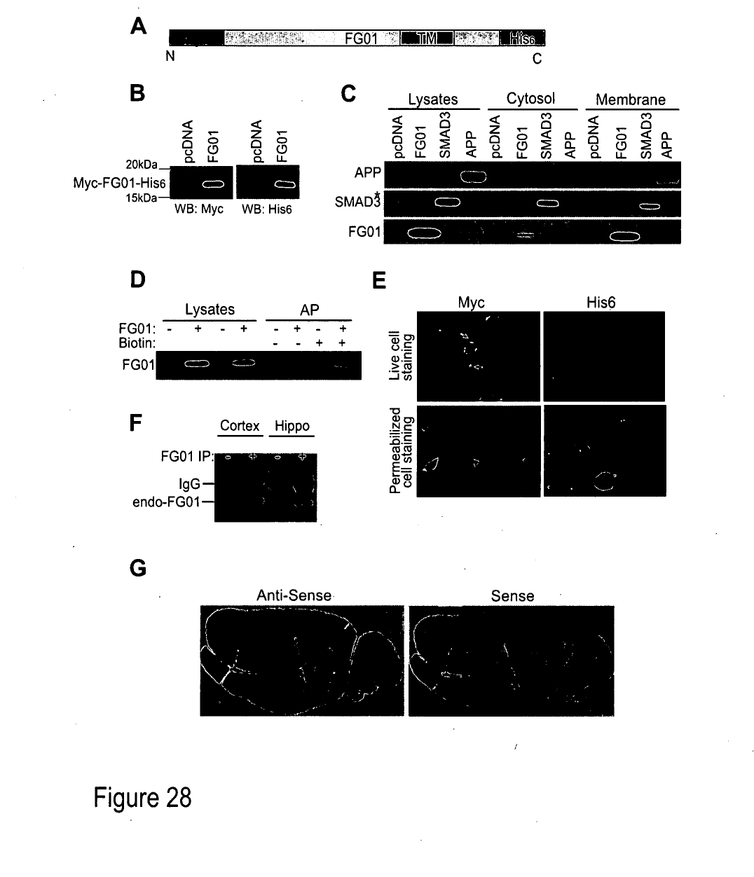

Bioinformatics analysis using the FG01 amino acid sequence predicted a helical transmembrane domain near the C-terminus but no obvious signal peptide sequence. A vector expressing recombinant FG01 was constructed with a Myc tag at the N-terminus and a His₆ tag at the C-terminus (Myc-FG01-His₆, FIG. 28A). Both Myc and His₆ antibodies recognized a product of approximately 17 kDa in transfected Myc-FG01-His₆ cells, consistent with the predicted molecular weight, indicating that there is no cleavable signal peptide sequence within FG01 (FIG. 28B). Furthermore, after transfection of Myc-FG01-His₆ vector into N2a cells, fractionation of cell lysates into cytosolic and membrane components indicated that the majority of FG01 protein was located in membrane fractions (FIG. 28C). Biotinylation assays also revealed that FG01 was delivered to the cell surface (FIG. 28D). To determine FG01 topology, N2a cells were transfected with the Myc-FG01-His₆ vector and immunostained either live cells or cells after permeabilization, using antibodies against Myc or His₆. The results show that although both antibodies were immunoreactive in permeablized cells, only the Myc antibody positively stains the membranes of live cells, whereas the His₆ antibody does not, indicating that the FG01 N-terminus is extracellular (FIG. 28E). Immunoprecipitation combined with live-immunostaining also confirmed the type III transmembrane topology of FG01. N2a cells were transfected with Myc-FG01-His₆. Live cells were incubated with Myc or His₆ antibody, or mouse IgG, at 4° C. for 2 hrs. Additionally, cells were incubated with Myc or His₆ antibodies in the presence of 0.1% Triton X-100, 0.2% Triton X-100, or 0.1% Triton X-100 and 0.05% Deoxycholate. After incubation, cells were washed with ice-cold phosphate buffered saline, collected and lysed. Equal lysate volumes were incubated with protein A sepharose beads at 4° C. overnight, and immunoprecipitated proteins were subjected to SDS-PAGE and Western blot with Myc or His6 antibody. 5% of lysates used for immunoprecipitation were used as input.

An antibody against the N-terminus of FG01 was derived. Mouse N2a cells were transfected with Myc-tagged FG01. Equal lysate volumes were immunoprecipitated with pre-immune serum, Myc antibody, or FG01 antibody, followed by Western blot with Myc or FG01 antibody. Using this antibody for immunoprecipitation followed by Western blot analysis, expression of FG01 in both cortex and hippocampus was confirmed (FIG. 28F). In situ hybridization analysis of fg01 expression in mouse brain revealed that fg01 is indeed expressed primarily in hippocampus, dentate gyrus, and cortex (FIG. 28G).

d. FG01 Overexpression Reduces Aβ Generation, GSK-3 Activity and Tau Phosphorylation

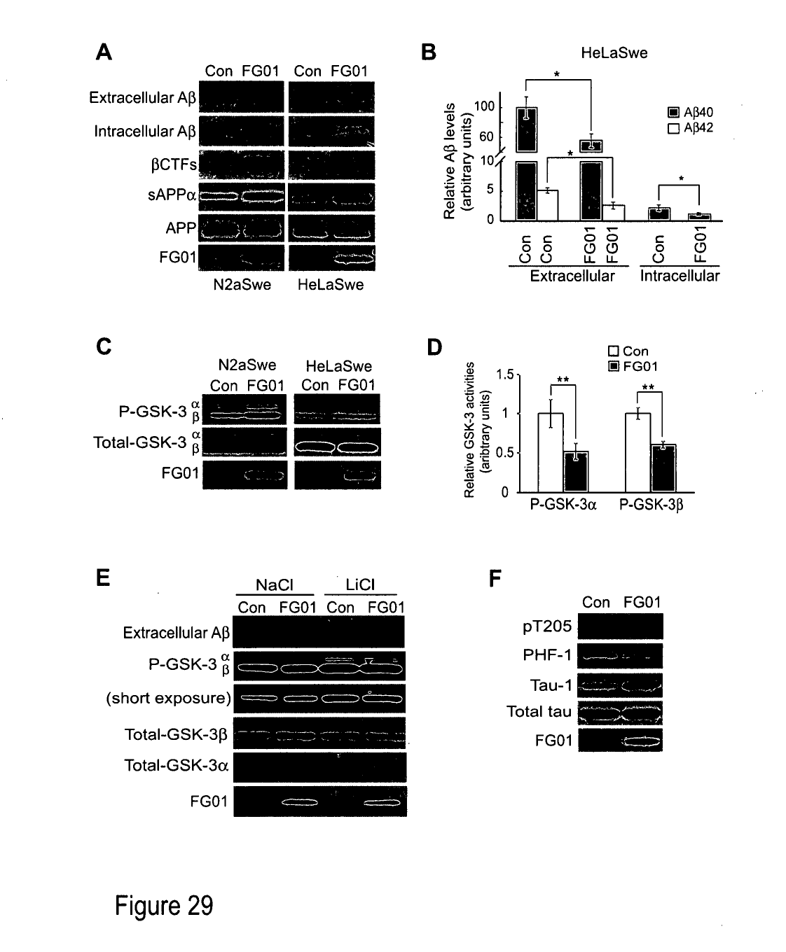

The FG01 RHKO cell clone exhibits increased APP βCTF accumulation and reduced Aβ production (FIG. 27C), indicating that FG01 regulates APP processing/Aβ generation. To corroborate this, FG01 was over expressed in N2aSwe cells. The results showed that, although the levels of total APP were not affected, the levels of extracellular and intracellular Aβ were dramatically reduced by FG01 overexpression, and levels of βCTF and sAPPα were greatly increased (FIG. 29A, left panels). When human HeLa cells stably expressing the human APP Swedish mutation (HeLaSwe) were transfected with mouse FG01, it was also observed that reduced Aβ generation and increased accumulation of βCTF and sAPPα (FIG. 29A, right panels). ELISA analysis confirmed that both Aβ40 and Aβ42 levels were significantly reduced following FG01 expression in HeLaSwe cells (FIG. 29B). These data demonstrated that mouse FG01 can function not only in mouse cells, but also in human cells.

To determine whether FG01 reduces Aβ generation by modulating β-secretase activities, β-secretase (BACE1) activity and the protein level of BACE1 in vitro in FG01-overexpressing cells was examined and both were unchanged. FG01 cell clones identified by RHKO were treated with DMSO (control) or 2 μg/ml doxycycline for 72 hrs. In some experiments, N2a cells were transfected with control vector (Con) or FG01 vector for 48 hrs. Cell lysates were assayed for β-secretase activity. FG01 was transfected into N2a cells and equal protein lysates were subjected to SDS-PAGE and Western blot with antibodies against BACE1 (Yan et al., 2001), nicastrin (Leem et al., 2002) or Myc. APP βCTF accumulation can also be attributed to a decrease in γ-secretase-mediated cleavage.

Cleavage of Notch, another important γ-secretase substrate (Kopan and Goate, 2000), was not altered by FG01 overexpression. N2a cells were transfected with Myc-tagged Notch1 NΔE construct. After equal splitting, cells were transfected with control vector (Con) or FG01. Cell lysates were subjected to SDS-PAGE and Western blot with the Myc antibody (recognizing both exogenous FG01 and Notch NΔE) and with an NICD-specific antibody. Protein levels of nicastrin, an important component of the γ-secretase complex, were also not affected by FG01 overexpression.

Although the possibility that FG01 may modulate substrate specificity or accessibility to γ-secretase can not be excluded, it is equally possible that FG01 regulates other proteins/pathways functioning in APP processing and Aβ generation. One of those could be GSK-3, which has been shown to affect Aβ generation (without affecting γ-secretase-mediated Notch cleavage) and tau phosphorylation (Phiel et al., 2003; Takashima et al., 1995). Thus, the activities of GSK-3α/β were studied in the presence or absence of FG01 overexpression by examining levels of phospho-GSK-3α (Ser 21) and phospho-GSK-3β (Ser 9) (which represent inactivated forms of GSK-3) and by in vitro kinase assays. In cells overexpressing FG01 it was observed that GSK-3α/β was more highly phosphorylated at these sites in mouse N2aSwe (FIG. 29C, left panels), rat PC12, and human HeLaSwe (FIG. 29C, right panels) and HEK293 cells, indicating that FG01 can decrease GSK-3 kinase activity in cells of various types and species including human. In vitro kinase assays also demonstrated an approximately 50% decrease in GSK-3α and a 40% decrease in GSK-3β activities in FG01-overexpressing cells (FIG. 29D). Remarkably, in the presence of lithium, a general inhibitor of both GSK-3α and GSK-3β, FG01 overexpression could not further reduce GSK-3α/β activity or Aβ production (FIG. 29E). These data indicate that FG01 reduces Aβ generation by down-regulating GSK-3 activity.

GSK-3 is a major kinase that phosphorylates tau in AD (Flaherty et al., 2000). Hence, whether FG01 affects tau phosphorylation by inhibiting GSK-3 was examined. N2aSwe cells were cotransfected with the human tau splice variant T40 and with FG01 (or control vectors), and examined tau phosphorylation at the threonine 205 site (Thr205) and the PHF-1 tau epitope sites (serine 396 and serine 404), which are GSK-3 phosphorylation targets, and at the major paired helical filament (PHF) sites found in NFTs. Overexpression of FG01 significantly decreased tau phosphorylation at these GSK-3 target sites and increased unphosphorylated tau levels without affecting total tau levels (FIG. 29F). These results indicate a role for FG01 in reducing tau phosphorylation, in addition to its effect on Aβ generation. Protein levels and activity of CDK5, another kinase mediating tau phosphorylation in AD (Flaherty et al., 2000), was analyzed following FG01 overexpression. Little change was observed, indicating that CDK5 is not involved in FG01-regulated tau phosphorylation. N2a cells were transfected with FG01 or a control vector (Con). Equal cell lysate proteins were used for assaying in vitro CDK5 activity.

e. FG01 Interacts with Adenylate Cyclases to Upregulate cAMP Levels and PKA Activity

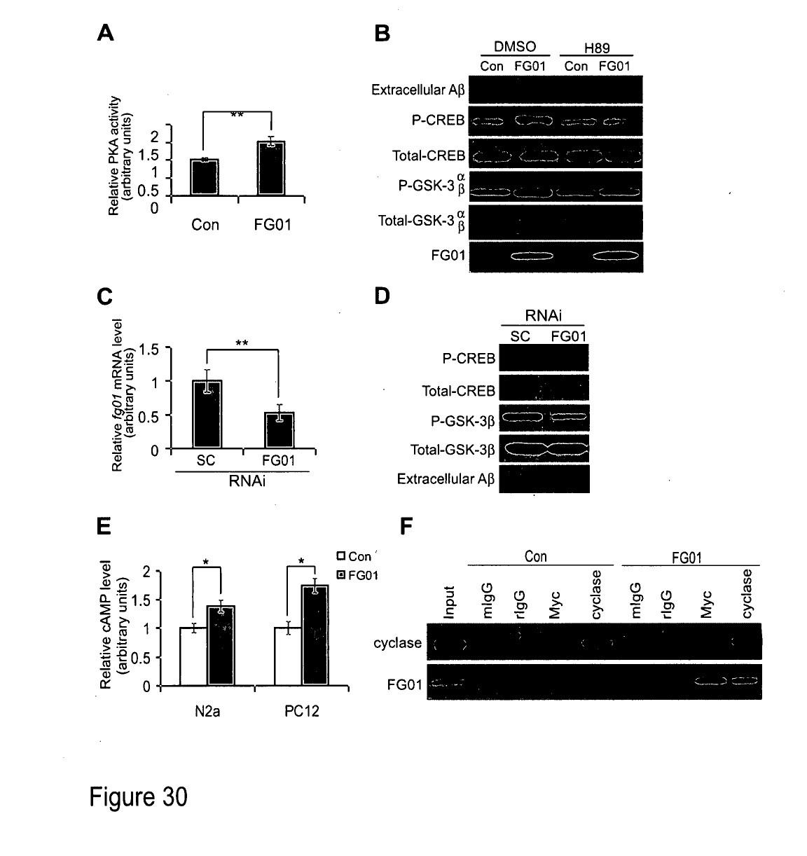

Inhibition of GSK-3 activity via phosphorylation of serine 21 in GSK-3α and serine 9 in GSK-3β can be mediated by protein kinase A (PKA) (Fang et al., 2000), so whether FG01 regulates PKA activity was studied. In vitro kinase assays revealed that FG01-transfected cells had significantly more PKA activity than control cells (FIG. 30A), consistent with the observation that FG01 overexpression increased phosphorylation of CREB, a PKA substrate (FIG. 30B). In addition, FG01 failed to inhibit GSK-3 activity and Aβ generation when PKA activity was suppressed by the specific inhibitor H89 (FIG. 30B). Furthermore, downregulation of endogenous FG01 expression in N2aSwe cells by RNA interference (FIG. 30C) dramatically reduced CREB phosphorylation, and increased GSK-3 activity and Aβ generation (FIG. 30D). These data indicate that FG01's effects on GSK-3 and Aβ generation require PKA activation. Moreover, increased sAPPα secretion upon FG01 overexpression (FIG. 29A) is also likely due to PKA activation, because PKA can stimulate budding of APP-containing vesicles from the Trans-Golgi Network (TGN) and facilitates sAPPα generation (Xu et al., 1996).

Since cAMP binds to and activates PKA (Taylor et al., 2008), whether FG01 overepxrerssion had any effect on cAMP levels was investigated. It was found that FG01 overexpression significantly increased cAMP levels in both mouse N2a (38%) and rat PC12 (75%) cells (FIG. 30E). The potential interaction between FG01 and adenylate cyclases, enzymes responsible for cAMP synthesis (Kamenetsky et al., 2006), was next examined. Co-immunoprecipitation studies showed that FG01 interacts with both overexpressed (data not shown) and endogenous (FIG. 30F) adenylate cyclases in N2a cells overexpressing FG01, indicating a modulation of enzymatic activity consistent with cAMP production.

To confirm that it is indeed FG01 protein rather than fg01 mRNA that mediates these effects, an fg01 cDNA mutant vector was constructed with a stop codon at the beginning of the protein-coding region. Cells transfected with this mutant vector showed mRNA expression (detected by RT-PCR) but no protein expression of the mutant fg01. In addition, overexpression of this mutant FG01 did not affect APP processing/Aβ generation, tau phosphorylation, and the activity of GSK-3 and PKA, excluding any potential RNA interfering effects arising from anti-sense interaction with RPS23 RNA.

f. Overexpression of FG01 Reduces Aβ Generation, GSK-3 Activity and Tau Phosphorylation in the Brain of the Triple Transgenic AD Mice

To validate FG01 function in vivo, an FG01 transgenic mouse model specifically overexpressing Myc-tagged FG01 in the brain was generated. A transgenic expression cassette driven by the human Thy-1 promoter was microinjected into C57B16 mice and primers specifically amplifying exogenous Myc-FG01 were used to genotype transgenic mice. Genomic DNA extracted from mouse tail samples was genotyped by PCR using Genotyping-P5 and Genotyping-P3 primers located in the human Thy1 and SV40 polyA regions, respectively. Reverse transcription-PCR revealed that mRNA of the exogenous gene was indeed expressed in transgenic mouse brains. Total RNA was extracted from the brain of an FG01 transgenic mouse (1) and of a littermate control (0), followed by RT-PCR with the RT-P5 primer located within the FG01-coding region and the Genotyping-P3 primer for detecting Myc-FG01 mRNA. Immunoprecipitation/Western blot confirmed that exogenous Myc-tagged FG01 protein was expressed in brain tissues of transgenic mice. Brain samples from FG01 transgenic (Tg) mice and littermate controls (Non-Tg) were analyzed for levels of phosphorylated/total CREB, phosphorylated/total GSK-3, and various tau forms by direct Western blot. Myc-FG01 was detected by immunoprecipitation-Western blot using the Myc antibody. Two mouse lines with similar exogenous FG01 expression levels were generated and the results obtained from the two lines (including their crossing with 3×Tg mice as described below) were similar. The results showed that levels of phosphorylated and therefore inactive GSK-3α/β were increased, accompanied by reduced GSK-3β activity in FG01 transgenic mouse brain. Brain lysates from FG01-Tg and Non-Tg mice were analyzed for GSK-3β activity. Results were normalized to those of Non-Tg samples. Increased CREB phosphorylation indicative of upregulated PKA activity, as well as decreased phosphorylation of endogenous mouse brain tau, was also seen in FG01 transgenic mouse brain. In addition, observation detected no obvious aberrant behavioral phenotypes in the FG01 transgenic mice.

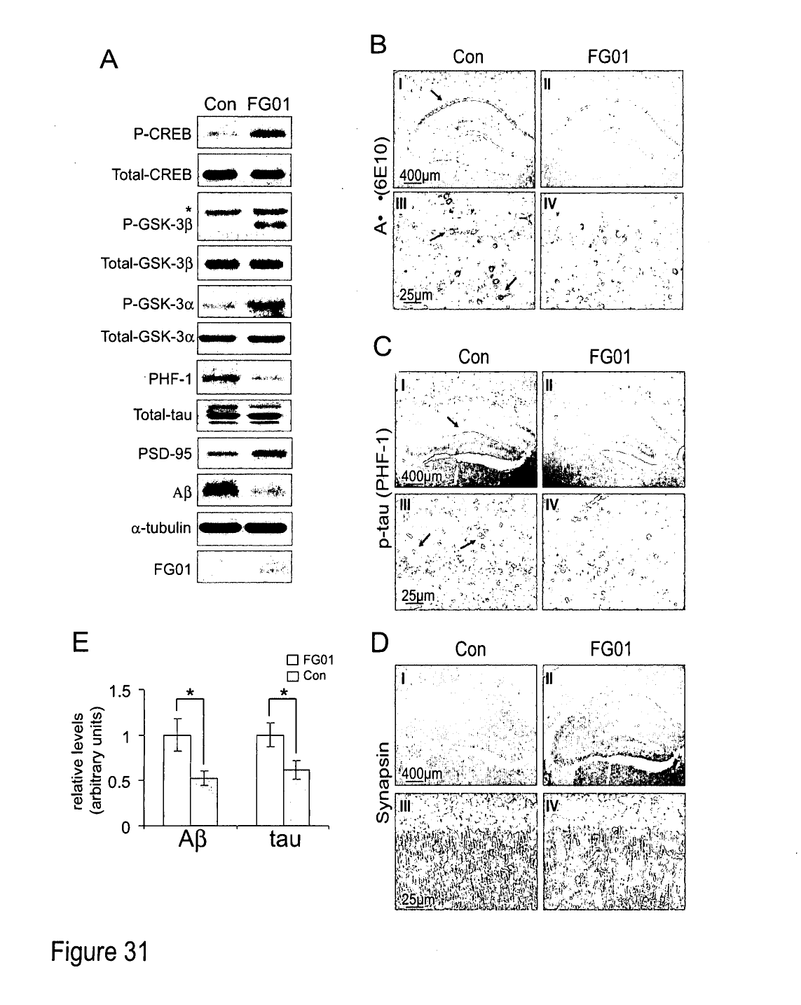

FG01 transgenic mice were crossed with triple transgenic (3×Tg) AD mice harboring mutations in human APP, tau and presenilin 1 (PS1) genes (Oddo et al., 2003). As expected, FG01 overexpression in the 3×Tg mice dramatically increased PKA activity and reduced GSK-3 activity, tau phosphorylation, and Aβ generation in mouse brains (FIG. 31A). Consistently, the numbers of both Aβ-immunostaining-positive and phosphorylated tau-immunostaining-positive neurons in the 3×Tg mouse brain (both hippocampus and cortex) were significantly decreased following FG01 overexpression (FIGS. 31B, 31C and 31E). Interestingly, levels of the synaptic marker PSD-95 were markedly increased following FG01 overexpression in 3×Tg mouse brains (FIG. 31A). Immunostaining of synapsin, another synaptic marker, also revealed much higher immunoreactivity in the hippocampus of 3×Tg mouse brains with FG01 overexpression than that seen in 3×Tg mice without FG01 (FIG. 31D). These results imply that FG01 may rescue synapse impairment seen in 3×Tg mice (Oddo et al., 2003), in addition to its effects on reducing Aβ generation and tau phosphorylation.

iii. Discussion

Using the RHKO assay to screen for genes involved in regulating Aβ generation, the functional retroposed fg01 gene on mouse chromosome 8 was identified. The FG01 protein is a type III transmembrane protein and is expressed in the brain. Compelling evidence was generated to show that FG01 overexpression can reduce both Aβ generation and tau phosphorylation, two major pathological hallmarks of AD. The results also reveal the underlying mechanism, i.e. FG01 interacts with adenylate cyclases to upregulate cAMP levels, which activates PKA activity, hence inhibiting GSK-3 activity, tau phosphorylation and Aβ generation.

Sequence analyses demonstrated that fg01 originated through retroposition of mouse RPS23, which recruited regulatory units and additional protein encoding fragments at the retroposition site and became functional (FIG. 25). The reversal in transcriptional direction of fg01 relative to the parental RPS23 gene explains why there is no protein sequence similarity between FG01 and RPS23. RPS23 belongs to the ribosomal protein family and is highly conserved among species (Hori et al., 1993). Since human ribosomal protein genes have been found to generate a large number of processed pseudogenes through retroposition (Zhang et al., 2002), there is a possibility that human RPS23 may have also retroposed in humans and generated new functional genes with orientations and functions similar to that of fg01. However, although several human RPS23 retroposition sites in the human genome were identified, neither computational gene prediction nor RT-PCR with primers binding adjacent regions of these human RPS23 retroposition sites revealed any fg01-like genes, implying that the possibility for humans to possess functional fg01 homologs may be low.

During aging, humans are susceptible to AD pathogenesis, typically characterized by Aβ overproduction/aggregation and tau hyperphosphorylation. In contrast, wild type mice rarely develop AD pathologies (De Strooper et al., 1995; Jankowsky et al., 2007; Johnstone et al., 1991). The differences in AD susceptibility between humans and mice have been attributed to the sequence disparity between human and mouse Aβ (and possibly tau) that underlie different aggregation properties (De Strooper et al., 1995; Jankowsky et al., 2007; Johnstone et al., 1991), to the short lifespan of mice relative to humans (Jankowsky et al., 2004; Jankowsky et al., 2007), and to the differences in processing of human and mouse APP by BACE1 (Cai et al., 2001). A lack of fg01 homologs in humans provides an alternative explanation, i.e. some genetic factors in mice, such as FG01, protect them against an AD-like disease by preventing Aβ over-production and tau hyperphosphorylation.

Mouse FG01 also exerts its functions in human cells, indicating that FG01-mediated signaling pathways are active in humans. Thus, FG01 can be used for combating AD and other diseases such as cancer and diabetes, in which the PKA and GSK-3 signaling pathways are crucially involved (Martinez et al., 2002; Naviglio et al., 2009).

### H. REFERENCES

Ancolio et al. (1999). Unusual phenotypic alteration of beta amyloid precursor protein (betaAPP) maturation by a new Val-715--> Met betaAPP-770 mutation responsible for probable early-onset Alzheimer's disease. Proc. Natl. Acad. Sci. USA 96, 4119-4124.
Anderton, B. H. et al. Sites of phosphorylation in tau and factors affecting their regulation. Biochem Soc Symp, 73-80 (2001).
Babushok et al. (2007). Current topics in genome evolution: molecular mechanisms of new gene formation. Cell Mol. Life. Sci. 64, 542-554.
Baek, S. H. et al. Exchange of N-CoR corepressor and Tip60 coactivator complexes links gene expression by NF-kappaB and beta-amyloid precursor protein. Cell 110, 55-67 (2002).
Barelli et al. (1997). Characterization of new polyclonal antibodies specific for 40 and 42 amino acid-long amyloid beta peptides: their use to examine the cell biology of presenilins and the immunohistochemistry of sporadic Alzheimer's disease and cerebral amyloid angiopathy cases. Mol. Med. 3, 695-707.
Buee et al. (2000). Tau protein isoforms, phosphorylation and role in neurodegenerative disorders. Brain Res. Brain Res. Rev. 33, 95-130.
Burge and Karlin, (1997). Prediction of complete gene structures in human genomic DNA. J. Mol. Biol. 268, 78-94.
Burge and Karlin, (1998). Finding the genes in genomic DNA. Curr. Opin. Struct. Biol. 8, 346-354.
Buxbaum et al. (1994). Calcium regulates processing of the Alzheimer amyloid protein precursor in a protein kinase C-independent manner. Proc. Natl. Acad. Sci. USA 91, 4489-4493.
Cai et al. (2001). BACE1 is the major beta-secretase for generation of Abeta peptides by neurons. Nat. Neurosci. 4, 233-234.
Cao, X. & Sudhof, T. C. A transcriptionally [correction of transcriptively] active complex of APP with Fe65 and histone acetyltransferase Tip60. Science 293, 115-20 (2001).
Capell, A. et al. Maturation and pro-peptide cleavage of beta-secretase. J Biol Chem 275, 30849-54 (2000).
Chang, K. A. & Suh, Y. H. Pathophysiological roles of amyloidogenic carboxy-terminal fragments of the beta-amyloid precursor protein in Alzheimer's disease. J Pharmacol Sci 97, 461-71 (2005).
Chang, K. A. et al. Phosphorylation of amyloid precursor protein (APP) at Thr668 regulates the nuclear translocation of the APP intracellular domain and induces neurodegeneration. Mol Cell Biol 26, 4327-38 (2006).
Chen et al. (2000). Carboxyl-terminal fragments of Alzheimer beta-amyloid precursor protein accumulate in restricted and unpredicted intracellular compartments in presenilin 1-deficient cells. J. Biol. Chem. 275, 36794-36802.
Chen, F. et al. TMP21 is a presenilin complex component that modulates gamma-secretase but not epsilon-secretase activity. Nature 440, 1208-12 (2006).
Chen, M. & Fernandez, H. L. The Alzheimer's plaques, tangles and memory deficits may have a common origin. Part V: why is Ca2+ signal lower in the disease? Front Biosci 4, A9-15 (1999).
De Strooper et al. (1995). Production of intracellular amyloid-containing fragments in hippocampal neurons expressing human amyloid precursor protein and protection against amyloidogenesis by subtle amino acid substitutions in the rodent sequence. Embo J. 14, 4932-4938.
De Strooper, (2003). Aph-1, Pen-2, and Nicastrin with Presenilin generate an active gamma-Secretase complex. Neuron 38, 9-12.
De Strooper, B. et al. Deficiency of presenilin-1 inhibits the normal cleavage of amyloid precursor protein. Nature 391, 387-90 (1998).
Edbauer, D. et al. Reconstitution of gamma-secretase activity. Nat Cell Biol 5, 486-8 (2003).
Embi, N., Rylatt, D. B. & Cohen, P. Glycogen synthase kinase-3 from rabbit skeletal muscle. Separation from cyclic-AMP-dependent protein kinase and phosphorylase kinase. Eur J Biochem 107, 519-27 (1980).
Fang et al. (2000). Phosphorylation and inactivation of glycogen synthase kinase 3 by protein kinase A. Proc. Natl. Acad. Sci. USA 97, 11960-11965.
Flaherty et al. (2000). Phosphorylation of human tau protein by microtubule-associated kinases: GSK3beta and cdk5 are key participants. J. Neurosci. Res. 62, 463-472.
Frame and Cohen, (2001). GSK3 takes centre stage more than 20 years after its discovery. Biochem. J. 359, 1-16.
Francis, R. et al. aph-1 and pen-2 are required for Notch pathway signaling, gamma-secretase cleavage of betaAPP, and presenilin protein accumulation. Dev Cell 3, 85-97 (2002).
Gao, Y. & Pimplikar, S. W. The gamma-secretase-cleaved C-terminal fragment of amyloid precursor protein mediates signaling to the nucleus. Proc Natl Acad Sci USA 98, 14979-84 (2001).
Gasparini et al. (2001). Stimulation of beta-amyloid precursor protein trafficking by insulin reduces intraneuronal beta-amyloid and requires mitogen-activated protein kinase signaling. J. Neurosci. 21, 2561-2570.
Golde, T. E. & Eckman, C. B. Cholesterol modulation as an emerging strategy for the treatment of Alzheimer's disease. Drug Discov Today 6, 1049-1055 (2001).
Goutte, C., Tsunozaki, M., Hale, V. A. & Priess, J. R. APH-1 is a multipass membrane protein essential for the Notch signaling pathway in Caenorhabditis elegans embryos. Proc Natl Acad Sci USA 99, 775-9 (2002).
Grant, P., Sharma, P. & Pant, H. C. Cyclin-dependent protein kinase 5 (Cdk5) and the regulation of neurofilament metabolism. Eur J Biochem 268, 1534-46 (2001).
Greenfield, J. P. et al. Estrogen lowers Alzheimer beta-amyloid generation by stimulating trans-Golgi network vesicle biogenesis. J Biol Chem 277, 12128-36 (2002).
Greenfield, J. P., Gross, R. S., Gouras, G. K. & Xu, H. Cellular and molecular basis of beta-amyloid precursor protein metabolism. Front Biosci 5, D72-83 (2000).
Han et al. (2005). Suppression of cyclin-dependent kinase 5 activation by amyloid precursor protein: a novel excitoprotective mechanism involving modulation of tau phosphorylation. J. Neurosci. 25, 11542-11552.
Hanger, D. P., Hughes, K., Woodgett, J. R., Brion, J. P. & Anderton, B. H. Glycogen synthase kinase-3 induces Alzheimer's disease-like phosphorylation of tau: generation of paired helical filament epitopes and neuronal localisation of the kinase. Neurosci Lett 147, 58-62 (1992).
Hardy and Selkoe, (2002). The amyloid hypothesis of Alzheimer's disease: progress and problems on the road to therapeutics. Science 297, 353-356.
Hollis et al. (1982). Processed genes: a dispersed human immunoglobulin gene bearing evidence of RNA-type processing. Nature 296, 321-325.
Hong, M., Chen, D. C., Klein, P. S. & Lee, V. M. Lithium reduces tau phosphorylation by inhibition of glycogen synthase kinase-3. J Biol Chem 272, 25326-32 (1997).
Hori et al. (1993). A cDNA sequence of human ribosomal protein, homologue of yeast S28. Nucleic Acids Res. 21, 4394.
Hussain, I. et al. Identification of a novel aspartic protease (Asp 2) as beta-secretase. Mol Cell Neurosci 14, 419-27 (1999).
Imahori, K. & Uchida, T. Physiology and pathology of tau protein kinases in relation to Alzheimer's disease. J Biochem (Tokyo) 121, 179-88 (1997).
Jankowsky et al. (2004). Mutant presenilins specifically elevate the levels of the 42 residue beta-amyloid peptide in vivo: evidence for augmentation of a 42-specific gamma secretase. Hum. Mol. Genet. 13, 159-170.
Jankowsky et al. (2007). Rodent A beta modulates the solubility and distribution of amyloid deposits in transgenic mice. J. Biol. Chem. 282, 22707-22720.
Jeffs and Ashburner, (1991). Processed pseudogenes in Drosophila. Proc. Biol. Sci. 244, 151-159.
Johnstone et al. (1991). Conservation of the sequence of the Alzheimer's disease amyloid peptide in dog, polar bear and five other mammals by cross-species polymerase chain reaction analysis. Brain Res. Mol. Brain. Res. 10, 299-305.
Kaessmann et al. (2009). RNA-based gene duplication: mechanistic and evolutionary insights. Nat. Rev. Genet. 10, 19-31.
Kamenetsky et al. (2006). Molecular details of cAMP generation in mammalian cells: a tale of two systems. J. Mol. Biol. 362, 623-639.
Karin and Richards, (1982). Human metallothionein genes—primary structure of the metallothionein-II gene and a related processed gene. Nature 299, 797-802.
Kim, H. S. et al. C-terminal fragments of amyloid precursor protein exert neurotoxicity by inducing glycogen synthase kinase-3beta expression. Faseb J 17, 1951-3 (2003).
Kim, S. H. et al. Multiple effects of aspartate mutant presenilin 1 on the processing and trafficking of amyloid precursor protein. J Biol Chem 276, 43343-50 (2001).
Kimberly, W. T. et al. Gamma-secretase is a membrane protein complex comprised of presenilin, nicastrin, Aph-1, and Pen-2. Proc Natl Acad Sci USA 100, 6382-7 (2003).
Kopan and Goate, (2000). A common enzyme connects notch signaling and Alzheimer's disease. Genes Dev. 14, 2799-2806.
Kulic, L. et al. Separation of presenilin function in amyloid beta-peptide generation and endoproteolysis of Notch. Proc Natl Acad Sci USA 97, 5913-8 (2000).
Lee et al. (2001). Neurodegenerative tauopathies. Annu. Rev. Neurosci. 24, 1121-1159.
Leem et al. (2002). Presenilin 1 is required for maturation and cell surface accumulation of nicastrin. J. Biol. Chem. 277, 19236-19240.
Li, L. & Cohen, S, N. Tsg101: a novel tumor susceptibility gene isolated by controlled homozygous functional knockout of allelic loci in mammalian cells. Cell 85, 319-29 (1996).
Li, L., and Cohen, S, N. (1996). Tsg101: a novel tumor susceptibility gene isolated by controlled homozygous functional knockout of allelic loci in mammalian cells. Cell 85, 319-329.
Lin et al. (2007). Calnuc binds to Alzheimer's beta-amyloid precursor protein and affects its biogenesis. J. Neurochem. 100, 1505-1514.
Lin, X. et al. Human aspartic protease memapsin 2 cleaves the beta-secretase site of beta-amyloid precursor protein. Proc Natl Acad Sci USA 97, 1456-60 (2000).
Liu et al. (1999). Reversible tumorigenesis induced by deficiency of vasodilator-stimulated phosphoprotein. Mol. Cell. Biol. 19, 3696-3703.
Liu et al. (2000a (non) b (prov)). Antisense RNA-mediated deficiency of the calpain protease, nCL-4, in NIH3T3 cells is associated with neoplastic transformation and tumorigenesis. J. Biol. Chem. 275, 31093-31098.
Liu et al. (2000a (prov) b (non)). Neoplastic transformation and tumorigenesis associated with sam68 protein deficiency in cultured murine fibroblasts. J. Biol. Chem. 275, 40195-40201.
Liu, S. J. et al. Overactivation of glycogen synthase kinase-3 by inhibition of phosphoinositol-3 kinase and protein kinase C leads to hyperphosphorylation of tau and impairment of spatial memory. J Neurochem 87, 1333-44 (2003).
Liu, S. J. et al. Tau becomes a more favorable substrate for GSK-3 when it is prephosphorylated by PKA in rat brain. J Biol Chem 279, 50078-88 (2004).
Long et al. (2003). The origin of new genes: glimpses from the young and old. Nat. Rev. Genet. 4, 865-875.
Lovestone, S. et al. Alzheimer's disease-like phosphorylation of the microtubule-associated protein tau by glycogen synthase kinase-3 in transfected mammalian cells. Curr Biol 4, 1077-86 (1994).
Lovestone, S. et al. Lithium reduces tau phosphorylation: effects in living cells and in neurons at therapeutic concentrations. Biol Psychiatry 45, 995-1003 (1999).
Martinez et al. (2002). Glycogen synthase kinase 3 (GSK-3) inhibitors as new promising drugs for diabetes, neurodegeneration, cancer, and inflammation. Med. Res. Rev. 22, 373-384.
Mattson, M. P. et al. Evidence for excitoprotective and intraneuronal calcium-regulating roles for secreted forms of the beta-amyloid precursor protein. Neuron 10, 243-54 (1993).
Medina and Castro, (2008). Glycogen synthase kinase-3 (GSK-3) inhibitors reach the clinic. Curr. Opin. Drug Discov. Devel. 11, 533-543.
Mighell et al. (2000). Vertebrate pseudogenes. FEBS Lett. 468, 109-114.
Mills, J. & Reiner, P. B. Regulation of amyloid precursor protein cleavage. J Neurochem 72, 443-60 (1999).
Munoz-Montano, J. R., Moreno, F. J., Avila, J. & Diaz-Nido, J. Lithium inhibits Alzheimer's disease-like tau protein phosphorylation in neurons. FEBS Lett 411, 183-8 (1997).
Muresan, Z. & Muresan, V. Coordinated transport of phosphorylated amyloid-beta precursor protein and c-Jun NH2-terminal kinase-interacting protein-1. J Cell Biol 171, 615-25 (2005).
Naruse, S. et al. Effects of PS1 deficiency on membrane protein trafficking in neurons. Neuron 21, 1213-21 (1998).
Naviglio et al. (2009). Protein kinase A as a biological target in cancer therapy. Expert Opin. Ther. Targets 13, 83-92.
Oddo et al. (2003). Triple-transgenic model of Alzheimer's disease with plaques and tangles: intracellular Abeta and synaptic dysfunction. Neuron 39, 409-421.
Oddo, S., Billings, L., Kesslak, J. P., Cribbs, D. H. & LaFerla, F. M. Abeta immunotherapy leads to clearance of early, but not late, hyperphosphorylated tau aggregates via the proteasome. Neuron 43, 321-32 (2004).
Paglini, G. & Caceres, A. The role of the Cdk5—p35 kinase in neuronal development. Eur J Biochem 268, 1528-33 (2001).
Petit, A. et al. New protease inhibitors prevent gamma-secretase-mediated production of Abeta40/42 without affecting Notch cleavage. Nat Cell Biol 3, 507-11 (2001).
Phiel et al. (2003). GSK-3alpha regulates production of Alzheimer's disease amyloid-beta peptides. Nature 423, 435-439.
Phiel, C. J. & Klein, P. S. Molecular targets of lithium action. Annu Rev Pharmacol Toxicol 41, 789-813 (2001).
Phiel, C. J., Wilson, C. A., Lee, V. M. & Klein, P. S. GSK-3alpha regulates production of Alzheimer's disease amyloid-beta peptides. Nature 423, 435-9 (2003).
Puglielli, L. et al. Acyl-coenzyme A: cholesterol acyltransferase modulates the generation of the amyloid beta-peptide. Nat Cell Biol 3, 905-12 (2001).
Rogaev, E. I. et al. Familial Alzheimer's disease in kindreds with missense mutations in a gene on chromosome 1 related to the Alzheimer's disease type 3 gene. Nature 376, 775-8 (1995).
Rost et al. (2004). The PredictProtein server. Nucleic Acids Res. 32, W321-326.
Ryan, K. A. & Pimplikar, S. W. Activation of GSK-3 and phosphorylation of CRMP2 in transgenic mice expressing APP intracellular domain. J Cell Biol 171, 327-35 (2005).
Schroeter et al. (1998). Notch-1 signalling requires ligand-induced proteolytic release of intracellular domain. Nature 393, 382-386.
Sengupta, A., Wu, Q., Grundke-Iqbal, I., Iqbal, K. & Singh, T. J. Potentiation of GSK-3-catalyzed Alzheimer-like phosphorylation of human tau by cdk5. Mol Cell Biochem 167, 99-105 (1997).
Sherrington, R. et al. Cloning of a gene bearing missense mutations in early-onset familial Alzheimer's disease. Nature 375, 754-60 (1995).
Sinha, S. et al. Purification and cloning of amyloid precursor protein beta-secretase from human brain. Nature 402, 537-40 (1999).
Sisodia, S. S. Alzheimer's disease: perspectives for the new millennium. J Clin Invest 104, 1169-70 (1999).
Sperber, B. R., Leight, S., Goedert, M. & Lee, V. M. Glycogen synthase kinase-3 beta phosphorylates tau protein at multiple sites in intact cells. Neurosci Lett 197, 149-53 (1995).
Stambolic, V., Ruel, L. & Woodgett, J. R. Lithium inhibits glycogen synthase kinase-3 activity and mimics wingless signalling in intact cells. Curr Biol 6, 1664-8 (1996).
Struhl, G. & Adachi, A. Requirements for presenilin-dependent cleavage of notch and other transmembrane proteins. Mol Cell 6, 625-36 (2000).
Sumioka, A. et al. Role of 14-3-3gamma in FE65-dependent gene transactivation mediated by the amyloid beta-protein precursor cytoplasmic fragment. J Biol Chem 280, 42364-74 (2005).
Takashima et al. (1995). Amyloid beta peptide induces cytoplasmic accumulation of amyloid protein precursor via tau protein kinase I/glycogen synthase kinase-3 beta in rat hippocampal neurons. Neurosci. Lett. 198, 83-86.
Takashima, A. et al. Presenilin 1 associates with glycogen synthase kinase-3beta and its substrate tau. Proc Natl Acad Sci USA 95, 9637-41 (1998).
Takashima, A. GSK-3 is essential in the pathogenesis of Alzheimer's disease. J Alzheimers Dis 9, 309-17 (2006).
Takasugi, N. et al. The role of presenilin cofactors in the gamma-secretase complex. Nature 422, 438-41 (2003).
Tanzi and Bertram, (2005). Twenty years of the Alzheimer's disease amyloid hypothesis: a genetic perspective. Cell 120, 545-555.
Taylor et al. (2008). Signaling through cAMP and cAMP-dependent protein kinase: diverse strategies for drug design. Biochim. Biophys. Acta 1784, 16-26.
Telese, F. et al. Transcription regulation by the adaptor protein Fe65 and the nucleosome assembly factor SET. EMBO Rep 6, 77-82 (2005).
Tesco, G. & Tanzi, R. E. GSK3 beta forms a tetrameric complex with endogenous PS1-CTF/NTF and beta-catenin. Effects of the D257/D385A and FAD-linked mutations. Ann N Y Acad Sci 920, 227-32 (2000).
Ueda et al. (1982). Long terminal repeat-like elements flank a human immunoglobulin epsilon pseudogene that lacks introns. Embo J. 1, 1539-1544.
Vassar, R. et al. Beta-secretase cleavage of Alzheimer's amyloid precursor protein by the transmembrane aspartic protease BACE. Science 286, 735-41 (1999).
Vinckenbosch et al. (2006). Evolutionary fate of retroposed gene copies in the human genome. Proc. Natl. Acad. Sci. USA 103, 3220-3225.
von Rotz, R. C. et al. The APP intracellular domain forms nuclear multiprotein complexes and regulates the transcription of its own precursor. J Cell Sci 117, 4435-48 (2004).
Wang et al. (2006). Transcriptional regulation of APH-1A and increased gamma-secretase cleavage of APP and Notch by HIF-1 and hypoxia. Faseb J. 20, 1275-1277.
Xu et al. (1996). Metabolism of Alzheimer beta-amyloid precursor protein: regulation by protein kinase A in intact cells and in a cell-free system. Proc. Natl. Acad. Sci. USA 93, 4081-4084.
Xu et al. (1997). Generation of Alzheimer beta-amyloid protein in the trans-Golgi network in the apparent absence of vesicle formation. Proc. Natl. Acad. Sci. USA 94, 3748-3752.
Xu et al. (1998). Estrogen reduces neuronal generation of Alzheimer beta-amyloid peptides. Nat. Med. 4, 447-451.
Xu, H., Greengard, P. & Gandy, S. Regulated formation of Golgi secretory vesicles containing Alzheimer beta-amyloid precursor protein. J Biol Chem 270, 23243-5 (1995).
Yan et al. (2001). The transmembrane domain of the Alzheimer's beta-secretase (BACE1) determines its late Golgi localization and access to beta-amyloid precursor protein (APP) substrate. J. Biol. Chem. 276, 36788-36796.
Yan, R. et al. Membrane-anchored aspartyl protease with Alzheimer's disease beta-secretase activity. Nature 402, 533-7 (1999).
Yang, D. S. et al. Mature glycosylation and trafficking of nicastrin modulate its binding to presenilins. J Biol Chem 277, 28135-42 (2002).
York et al. (2000). Role of phosphoinositide 3-kinase and endocytosis in nerve growth factor-induced extracellular signal-regulated kinase activation via Ras and Rap1. Mol. Cell Biol. 20, 8069-8083.
Yu, G. et al. Nicastrin modulates presenilin-mediated notch/glp-1 signal transduction and betaAPP processing. Nature 407, 48-54 (2000).
Zhang and Xu, (2007). Molecular and Cellular Mechanisms for Alzheimer's Disease: Understanding APP Metabolism. Curr. Mol. Med. 7, 687-696.
Zhang et al. (2002). Identification and analysis of over 2000 ribosomal protein pseudogenes in the human genome. Genome Res. 12, 1466-1482.
Zhang et al. (2004). Comparative analysis of processed pseudogenes in the mouse and human genomes. Trends Genet. 20, 62-67.
Zhang et al. (2007). Hypoxia-inducible factor 1alpha (HIF-1alpha)-mediated hypoxia increases BACE1 expression and beta-amyloid generation. J. Biol. Chem. 282, 10873-10880.

### I. SEQUENCES

SEQ ID NO: 1

ATGCAGAGCCAACAGAGAAACATTGGCTACTTTAACCACCTTAAAGCGGA
CTCCAGGAATATCACCTACAGCATGACCTTTTCGACGAAATCCAGCAACC
AGAACTTCATCATTTTCCTCAATGAAGTTCAGGCAGCCATCATTGGGCAC
GAACGCTGTGATCTTCTTCCCGTTCTTAATGAGCTGCACCCTGACGCACT
TCCTGATGGCAGAATTTGGCTGTTTGGCCTCAACCCCTACTTTTTCCAGC
ACAATTCCCTTTGCATGAGAGGCACCCCCAAACGGATTGGCCTTCAGGGC
TGTGCCCAAGTGGGCTTTCTTGTACTGTTTGTCATGCCACTTCTGATCCC
GTCGGTGACTGCGGAGCTTCCGGGCAGTTCGGAGACCACGACACTTGCCC
ATCTTGCCGGCGCCACGGGCCCCTAA

SEQ ID NO: 2

MQSQQRNIGYFNHLKADSRNITYSMTFSTKSSNQNFIIFLNEVQAAIIGH
ERCDLLPVLNELHPDALPDGRIWLFGLNPYFFQHNSLCMRGTPKRIGLQG
CAQVGFLVLFVMPLLIPSVTAELPGSSETTTLAHLAGATGP

SEQ ID NO: 3

RRRRRRRRR

SEQ ID NO: 4

RQPKIWFPNRRKPWKK

SEQ ID NO: 5

GRKKRRQRPPQ

SEQ ID NO: 6

RQIKIWFQNRRMKWKK

SEQ ID NO: 7

RQIAIWFQNRRMKWAA

SEQ ID NO: 8

RKKRRQRRR

SEQ ID NO: 9

TRSSRAGLQFPVGRVHRLLRK

SEQ ID NO: 10

GWTLNSAGYLLGKINKALAALAKKIL

SEQ ID NO: 11

KLALKLALKALKAALKLA

SEQ ID NO: 12

AAVALLPAVLLALLAP

SEQ ID NO: 13

VPMLKPMLKE

SEQ ID NO: 14

MANLGYWLLALFVTMWTDVGLCKKRPKP

SEQ ID NO: 15

LLIILRRRIRKQAHAHSK

SEQ ID NO: 16

KETWWETWWTEWSQPKKKRKV

SEQ ID NO: 17

RGGRLSYSRRRFSTSTGR

SEQ ID NO: 18

SDLWEMMMVSLACQY

SEQ ID NO: 19

TSPLNIHNGQKL

SEQ ID NO: 20

UACUGUUUGUCAUGCCACUUCUGAU

SEQ ID NO: 21

TGTTGCATACACATACATGC

SEQ ID NO: 22

TCATTAAGAACGGGAAGAAG

SEQ ID NO: 23

ttttttcagaatatgaaaatttattactgtgtttccattgtcaaatttta
tgatcttggcctttctttcttgcctttgtatagAGCCAACAGAGAAACAT
TGGCTACTTTAACCACCTTAAAGCGGACTCCAGGAATATCACCTACAGCA
TGACCTTTTCGACGAAATCCAGCAACCAGAACTTCATCATTTTCCTCAAT
GAAGTTCAGGCAGCCATCATTGGGCACGAACGCTGTGATCTTCTTCCCGT
TCTTAATGAGCTGCACCCTGACGCACTTCCTGATGGCAGAATTTGGCTGT
TTGGCCTCAACCCCTACTTTTTCCAGCACAATTCCCTTTGCATGAGAGGC
ACCCCCAAACGGATTGGCCTTCAGGGCTGTGCCCAAGTGGGCTTTCTTGT
ACTGTTTGTCATGCCACTTCTGATCCCGTCGGTGACTGCGGAGCTTCCGG
GCAGTTCGGAGACCACGACACTTGCCCATCTTGCCGGCGCCACGGGCCC

SEQ ID NO: 24

ttttttcagaatatgaaaatttattactgtgtttccattgtca
aattTTATGATCTTGGCCTTTCTTTCTTGCCTTTGTATAGAGCCAACAG
AGAAACATTGGCTACTTTAACCACCTTAAAGCGGACTCCAGGAATATCA
CCTACAGCATGACCTTTTCGACCAAATCCAGCAACCAGAACTTCATCAT
TTTCCTCAATGAAGTTCAGGCAGCCATCATTGGGCACGAACGCTGTGAT
CTTCTTCCCGTTCTTAATGAGCTGCACCCTGACGCACTTCCTGATGGCA
GAATTTGGCTGTTTGGCCTCAACCCCTACTTTTTCCAGCACAATTCCCT
TTGCATGAGAGGCACCCCCAAACGGATTGGCCTTCAGGGCTGTGCCCAA
GTGGGCTTTCTTGTACTGTTTGTCATGCCACTTCTGGTCCCGTCGGTGA
CTGCGGAGCTTCCGGGCAGTTCGGAGACCACGACACTTGCCCATcttgc
cggcgccacgggcccaagagaaag

SEQ ID NO: 25

GAGGCCCGAATTCTCGCCGCCACC

SEQ ID NO: 26

GGATGCGCGGATAGCCGCTGCTGG

SEQ ID NO: 27

AGAATTTGGCTGTTTGG

What is claimed is:

1. An isolated nucleic acid comprising a sequence at least 95% identical to SEQ ID NO:1 or a fragment thereof at least 24 residues in length, wherein the sequence encodes a polypeptide that binds adenylyl cyclase.

2. The isolated nucleic acid of claim 1, wherein the sequence encodes a polypeptide comprising the amino acid sequence SEQ ID NO:2, or a fragment thereof at least 8 residues in length.

3. The isolated nucleic acid of claim 2, wherein the amino acid sequence comprises amino acids 96 to 141 of SEQ ID NO:2.

4. The isolated nucleic acid of claim 1, wherein the sequence comprises SEQ ID NO:1, or a fragment thereof at least 24 residues in length.

5. The isolated nucleic acid of claim 1, wherein the nucleic acid hybridizes under stringent conditions to a hybridization probe consisting of the sequence SEQ ID NO:1 or the complement of SEQ ID NO:1.

6. An expression vector comprising the isolated nucleic acid of claim 1, operably linked to an expression control sequence.

7. The expression vector of claim 6, wherein the expression control sequence is a tissue specific promoter.

8. The expression vector of claim 6, wherein the expression control sequence is an inducible promoter.

9. A method comprising administering the expression vector of claim 6 to a subject.

10. A cultured cell, comprising the nucleic acid of claim 1 operably linked to an expression control sequence.

11. The cultured cell of claim 10, wherein the cell is a eukaryotic cell.

12. A method of making a polypeptide, the method comprising culturing the cell of claim 10 under conditions permitting translation of the isolated nucleic acid.

13. A purified polypeptide, comprising an amino acid sequence at least 95% identical to the sequence SEQ ID NO:2, or a fragment thereof at least 8 residues in length, wherein the polypeptide binds adenylyl cyclase.

14. The purified polypeptide of claim 13, wherein the amino acid sequence comprises the sequence SEQ ID NO:2, or a fragment thereof at least 8 residues in length.

15. The purified polypeptide of claim 13, wherein the amino acid sequence comprises at least 8 consecutive residues of SEQ ID NO:2.

16. A method of inhibiting GSK3 activity in a cell, comprising contacting the cell with an isolated nucleic acid comprising a sequence at least 95% identical to SEQ ID NO:1 or a fragment thereof at least 24 residues in length, operably linked to an expression control sequence, wherein the sequence encodes a polypeptide that binds adenylyl cyclase.

17. The method of claim 16, wherein the isolated nucleic acid encodes a polypeptide comprising the amino acid sequence SEQ ID NO:2, or a fragment thereof at least 8 residues in length.

18. The method of claim 17, wherein the amino acid sequence comprises amino acids 96 to 141 of SEQ ID NO:2.

19. The method of claim 16, wherein the isolated nucleic acid comprises SEQ ID NO:1, or a fragment thereof at least 24 residues in length.

20. The method of claim 16, wherein the isolated nucleic acid hybridizes under stringent conditions to a hybridization probe consisting of the sequence SEQ ID NO:1 or the complement of SEQ ID NO:1.

21. The method of claim 16, wherein the cell is in a subject, wherein the cell is contacted with the isolated nucleic acid by administering the isolated nucleic acid to the subject.

22. The method of claim 21, wherein the subject is diagnosed with, suspected of being afflicted with, or identified as being at risk of Alzheimer's disease, cancer, diabetes, or a combination.

23. The method of claim 21, wherein the subject is diagnosed with, suspected of being afflicted with, or identified as being at risk of Alzheimer's disease.

24. The method of claim 21, wherein the subject is diagnosed with, suspected of being afflicted with, or identified as being at risk of cancer.

25. The method of claim 21, wherein the subject is diagnosed with, suspected of being afflicted with, or identified as being at risk of diabetes.

26. The method of claim 21, wherein the subject is diagnosed with Alzheimer's disease, cancer, diabetes, or a combination.

27. The method of claim 21, wherein the subject is diagnosed with Alzheimer's disease.

28. The method of claim 21, wherein the subject is diagnosed with cancer.

29. The method of claim 21, wherein the subject is diagnosed with diabetes.

30. The method of claim 21, wherein the subject is suspected of being afflicted with or is identified as being at risk of Alzheimer's disease.

31. The method of claim 21, wherein amyloid beta (Aβ) generation is reduced in the subject.

32. A method of inhibiting GSK3 activity in a cell, comprising contacting the cell with a purified polypeptide comprising an amino acid sequence at least 95% identical to the sequence SEQ ID NO:2, or a fragment thereof at least 8 residues in length, wherein the polypeptide binds adenylyl cyclase.

33. The method of claim 32, wherein the amino acid sequence comprises amino acids 96 to 141 of SEQ ID NO:2.

34. The method of claim 32, wherein the cell is in a subject, wherein the cell is contacted with the purified polypeptide by administering the isolated nucleic acid to the subject.

35. The method of claim 34, wherein the subject is diagnosed with, suspected of being afflicted with, or identified as being at risk of Alzheimer's disease, cancer, diabetes, or a combination.

36. The method of claim 34, wherein the subject is diagnosed with, suspected of being afflicted with, or identified as being at risk of Alzheimer's disease.

37. The method of claim 34, wherein the subject is diagnosed with, suspected of being afflicted with, or identified as being at risk of cancer.

38. The method of claim 34, wherein the subject is diagnosed with, suspected of being afflicted with, or identified as being at risk of diabetes.

39. The method of claim 34, wherein the subject is diagnosed with Alzheimer's disease, cancer, diabetes, or a combination.

40. The method of claim 34, wherein the subject is diagnosed with Alzheimer's disease.

41. The method of claim 34, wherein the subject is diagnosed with cancer.

42. The method of claim 34, wherein the subject is diagnosed with diabetes.

43. The method of claim 34, wherein the subject is suspected of being afflicted with or is identified as being at risk of Alzheimer's disease.

44. The method of claim 34, wherein amyloid beta (Aβ) generation is reduced in the subject.

45. A method of treating a subject with Alzheimer's disease, cancer, or diabetes, the method comprising administering to the subject an expression vector encoding protein FG01, such that a therapeutically effective amount of protein FG01 is expressed in the subject.

46. A method of treating a subject with Alzheimer's disease, cancer, or diabetes, the method comprising administering to the subject a cell comprising an expression vector encoding protein FG01, such that a therapeutically effective amount of protein FG01 is expressed by the cell in the subject.

47. The method of claim 46, wherein the cell is a stem cell or progenitor cell.

48. The method of claim 46, wherein the cell is a cell from the subject.

49. A method comprising:
(a) contacting a purified polypeptide comprising an amino acid sequence at least 95% identical to the sequence SEQ ID NO:2, or a fragment thereof at least 8 residues in length, wherein the polypeptide binds adenylyl cyclase, with a test compound; and
(b) determining whether the test compound binds to the purified polypeptide, said binding being an indication that the test compound is a modulator of glycogen synthase kinase-3 (GSK-3).

50. The method of claim 49 further comprising, following determining whether the test compound binds to the purified polypeptide, making the test compound.

51. The method of claim 49 further comprising, following determining whether the test compound binds to the purified polypeptide, administering the test compound to a subject.

52. A method of identifying a modulator of glycogen synthase kinase-3 (GSK-3), the method comprising:
(a) providing a cell comprising a nucleic acid encoding FG01 operably linked to an expression control sequence, wherein the nucleic acid comprises a sequence at least 95% identical to SEQ ID NO:1, or a fragment thereof at least 24 residues in length, wherein the sequence encodes a polypeptide that binds adenylyl cyclase,
(b) contacting the cell with a test compound; and
(c) measuring expression of FG01, an increase or decrease in FG01 expression an indication that the compound is a modulator of GSK-3.

53. The method of claim 52 further comprising, following measuring expression of FG01, making the test compound.

54. The method of claim 52 further comprising, following measuring expression of FG01, administering the test compound to a subject.

55. A process for making a modulator of glycogen synthase kinase-3 (GSK-3), the method comprising manufacturing the compound identified in claim 49.

56. A process for making a modulator of glycogen synthase kinase-3 (GSK-3), the method comprising manufacturing the compound identified in claim 52.

57. A method of identifying a compound that binds to FG01, the method comprising:
(a) providing a cell expressing FG01;
(b) contacting the cell with a test compound; and
(c) determining whether the test compound binds to FG01.


SEQUENCE LISTING (27 sequences)

SEQ ID NO: 1 - DNA, 426 bp, Homo sapiens
  gene (1)..(426)
atgcagagccaacagagaaacattggctactttaaccaccttaaagcggactccaggaatatcacctacagcatgaccttttcgacgaaatccagcaaccagaacttcatcattttcctcaatgaagttcaggcagccatcattgggcacgaacgctgtgatcttcttcccgttcttaatgagctgcaccctgacgcacttcctgatggcagaatttggctgtttggcctcaacccctactttttccagcacaattccctttgcatgagaggcacccccaaacggattggccttcagggctgtgcccaagtgggctttcttgtactgtttgtcatgccacttctgatcccgtcggtgactgcggagcttccgggcagttcggagaccacgacacttgcccatcttgccggcgccacgggcccctaa

SEQ ID NO: 2 - protein, 141 aa, homo sapien
  PEPTIDE (1)..(141)
MQSQQRNIGYFNHLKADSRNITYSMTFSTKSSNQNFIIFLNEVQAAIIGHERCDLLPVLNELHPDALPDGRIWLFGLNPYFFQHNSLCMRGTPKRIGLQGCAQVGFLVLFVMPLLIPSVTAELPGSSETTTLAHLAGATGP

SEQ ID NO: 3 - protein, 9 aa, Unknown
  cellular internalization transporter
RRRRRRRRR

SEQ ID NO: 4 - protein, 16 aa, Drosophila
  UNSURE (1)..(16)
RQPKIWFPNRRKPWKK

SEQ ID NO: 5 - protein, 11 aa, Human immunodeficiency virus
  UNSURE (1)..(11)
GRKKRRQRPPQ

SEQ ID NO: 6 - protein, 16 aa, drosophila
  UNSURE (1)..(16)
RQIKIWFQNRRMKWKK

SEQ ID NO: 7 - protein, 16 aa, drosophila
  UNSURE (1)..(16)
RQIAIWFQNRRMKWAA

SEQ ID NO: 8 - protein, 9 aa, Human immunodeficiency virus
  PEPTIDE (1)..(9)
RKKRRQRRR

SEQ ID NO: 9 - protein, 21 aa, unknown
  cellular internalization transporter
TRSSRAGLQFPVGRVHRLLRK

SEQ ID NO: 10 - protein, 26 aa, unknown
  cellular internalization transporter
GWTLNSAGYLLGKINKALAALAKKIL

SEQ ID NO: 11 - protein, 18 aa, unknown
  cellular internalization transporter
KLALKLALKALKAALKLA

SEQ ID NO: 12 - protein, 16 aa, unknown
  cellular internalization transporter
AAVALLPAVLLALLAP

SEQ ID NO: 13 - protein, 10 aa, unknown
  cellular internalization transporter
VPMLKPMLKE

SEQ ID NO: 14 - protein, 28 aa, unknown
  cellular internalization transporter
MANLGYWLLALFVTMWTDVGLCKKRPKP

SEQ ID NO: 15 - protein, 18 aa, unknown
  cellular internalization transporter
LLIILRRRIRKQAHAHSK

SEQ ID NO: 16 - protein, 21 aa, unknown
  cellular internalization transporter
KETWWETWWTEWSQPKKKRKV

SEQ ID NO: 17 - protein, 18 aa, unknown
  cellular internalization transporter
RGGRLSYSRRRFSTSTGR

SEQ ID NO: 18 - protein, 15 aa, unknown
  cellular internalization transporter
SDLWEMMMVSLACQY

SEQ ID NO: 19 - protein, 12 aa, unknown
  cellular internalization transporter
TSPLNIHNGQKL

SEQ ID NO: 20 - RNA, 25 nt, homo sapien
  Unsure (1)..(25)
uacuguuugucaugccacuucugau

SEQ ID NO: 21 - DNA, 20 bp, homo sapien
  Unsure (1)..(20)
tgttgcatacacatacatgc

SEQ ID NO: 22 - DNA, 20 bp, homo sapien
  unsure (1)..(20)
tcattaagaacgggaagaag

SEQ ID NO: 23 - DNA, 499 bp, homo sapien
  gene (1)..(499)
ttttttcagaatatgaaaatttattactgtgtttccattgtcaaattttatgatcttggcctttctttcttgcctttgtatagagccaacagagaaacattggctactttaaccaccttaaagcggactccaggaatatcacctacagcatgaccttttcgacgaaatccagcaaccagaacttcatcattttcctcaatgaagttcaggcagccatcattgggcacgaacgctgtgatcttcttcccgttcttaatgagctgcaccctgacgcacttcctgatggcagaatttggctgtttggcctcaacccctactttttccagcacaattccctttgcatgagaggcacccccaaacggattggccttcagggctgtgcccaagtgggctttcttgtactgtttgtcatgccacttctgatcccgtcggtgactgcggagcttccgggcagttcggagaccacgacacttgcccatcttgccggcgccacgggccc

SEQ ID NO: 24 - DNA, 508 bp, Mus musculus
  gene (1)..(508)
ttttttcagaatatgaaaatttattactgtgtttccattgtcaaattttatgatcttggcctttctttcttgcctttgtatagagccaacagagaaacattggctactttaaccaccttaaagcggactccaggaatatcacctacagcatgaccttttcgaccaaatccagcaaccagaacttcatcattttcctcaatgaagttcaggcagccatcattgggcacgaacgctgtgatcttcttcccgttcttaatgagctgcaccctgacgcacttcctgatggcagaatttggctgtttggcctcaacccctactttttccagcacaattccctttgcatgagaggcacccccaaacggattggccttcagggctgtgcccaagtgggctttcttgtactgtttgtcatgccacttctggtcccgtcggtgactgcggagcttccgggcagttcggagaccacgacacttgcccatcttgccggcgccacgggcccaagagaaag

SEQ ID NO: 25 - DNA, 24 bp, homo sapien
  Unsure (1)..(24)
gaggcccgaattctcgccgccacc

SEQ ID NO: 26 - DNA, 24 bp, Simian virus 40
  Unsure (1)..(24)
ggatgcgcggatagccgctgctgg

SEQ ID NO: 27 - DNA, 17 bp, homo sapien
  Unsure (1)..(17)
agaatttggctgtttgg